(19) United States
(12) Patent Application Publication
(10) Pub. No.: US 2024/0131007 A1
(43) Pub. Date: Apr. 25, 2024

(54) METHODS AND COMPOSITIONS FOR PICORNAVIRUS ANTI-VIRAL AGENTS

(71) Applicant: University of Connecticut, Farmington, CT (US)

(72) Inventors: Simon J. White, Mansfield, CT (US); Matthew K. Hadden, Ellington, CT (US); Rishabh Kejriwal, Bassett, VA (US)

(21) Appl. No.: 18/480,268

(22) Filed: Oct. 3, 2023

Related U.S. Application Data
(60) Provisional application No. 63/412,658, filed on Oct. 3, 2022.

(51) Int. Cl. A61K 31/4184; A61K 31/4985; A61K 31/506; A61K 31/53; A61P 31/04

(52) U.S. Cl. CPC A61K 31/4184; A61K 31/4985; A61K 31/506; A61K 31/53; A61P 31/04

(57) ABSTRACT

Disclosed are methods and compositions for treating or preventing a disease, a disorder, or symptom associated with picornaviruses in a subject including a therapeutically effective amount of a compound of Formula I, Formula II, or a pharmaceutically acceptable salt thereof, wherein X, Y, L, A, B, A₁, A₂, A₃, R₁, R₂, R₃, R₄, R₇, R₈, R₉, and R₁₀ are as described herein.

### CROSS-REFERENCE TO RELATED APPLICATIONS

This reference claims priority to, and the benefit of, U.S. Provisional Patent Application No. 63/412,658, filed 3 Oct. 2022 and titled METHODS AND COMPOSITIONS FOR PICORNAVIRUS ANTI-VIRAL AGENTS, which is incorporated by reference herein in its entirety for all purposes.

### BACKGROUND OF THE INVENTION

Field of the Invention The subject disclosure relates to methods and compositions for picornavirus anti-viral agents. In particular, the disclosure relates to the use of picornavirus anti-viral agents that target the viral 2C protein.

Description of the Related Art Picornaviruses make up the family Picornaviridae, which is in the order Picornavirales and realm Riboviria. Of note, the Picornavirales order constitutes a group of positive-strand RNA viruses that are characterized by a set of highly conserved genes, including nonstructural protein 2C, which is an ATPase. Picronaviruses comprise a major group of viruses and include several important pathogens of humans and animals, and include the notable genera Enterovirus, which includes Rhinovirus and Poliovirus), Aphthovirus, Cardiovirus, and Hepatovirus A. Enteroviruses (EVs) comprise a major group of viruses within the family of picornaviruses. EVs are small, non-enveloped, single stranded positive sense RNA viruses forming a genus within the Picornaviridae family. Seven out of fifteen species of enteroviruses are human pathogens that cause a myriad diseases of the skin, respiratory, circulatory and nervous systems. Enterovirus A71 (EV-A71) is one of the common causative agents of hand-foot-and-mouth disease. Although predominantly a disease in children, this can also affect adults and pose a serious threat to immunocompromised people. In a relatively small number of infections, EV-A71 can lead to more serious complications such as meningitis, encephalitis and acute flaccid myelitis and in the Asia-Pacific region, EV-A71 outbreaks have had an overall mortality rate of 0.5 to 19%. The related EV-D68 is responsible for acute flaccid myelitis outbreaks in multiple countries, including the USA, where there have been 682 confirmed cases since 2014. Poliovirus causes paralytic poliomyelitis and although almost eradicated, it still poses a threat due to the reversion of vaccine-derived poliovirus to pathogenic virus shed by people immunized with these vaccines. There are no Federal Drug Administration (FDA) approved drugs available against these viruses and there is an unmet clinical need for broad-spectrum anti-viral agents that show activity against enteroviruses. Such broad-acting drugs would relieve the need to identify the specific virus and facilitate treatment for acute infections that have a relatively small therapeutic window.

### SUMMARY OF THE INVENTION

In an aspect, disclosed is a method of treating or preventing a disease, a disorder, or at least one symptom associated with a picornavirus infection in a subject, the method comprising:
providing and administering a therapeutically effective amount of a compound of Formula I, or a pharmaceutically acceptable salt thereof, or a composition comprising the same to the subject, wherein Formula I has the chemical structure:
wherein in Formula I,

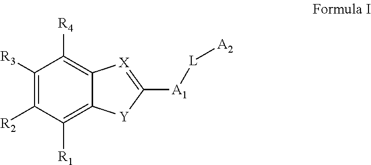

X is CR₅ or N;
Y is CR₅R₆, NR₅, O, or S;
L is a linker comprising O, NR₅, CONR₅, COO, COS, or S;
A₁ and A₂ are each independently an aryl (such as a monocyclic aryl or bicyclic aryl), a heteroaryl (such as a monoheteroaryl or bicyclic heteroaryl), an aryloxy (such as phenoxy), a thioaryl (such as thiophenyl), an alkyl (such as C₁-C₆ alkyl), an alkylene (such as a C₂₋₆ alkylene), an alkenyl (such as a C₂₋₆ alkenyl, ethenyl, or propenyl), an alkynyl (such as a C₂₋₆ alkynyl or acetylenyl), a haloalkyl (such as C₁-C₆ haloalkyl), an alkoxy (such as C₁-C₆ alkoxy), a cycloalkyl (such as a C₃-C₈ cycloalkyl group), a heterocycloalkyl (such as a 5-8 membered heterocycloalkyl), an alkylaryl (such as a C₁₋₃ alkyl-C₈₋₁₁ aryl, C₁₋₃ alkyl-C₆ aryl, C₁₋₃ alkyl-C₈ aryl, or C₁₋₃ alkyl-C₁₁ aryl), an alkylheteroaryl (such as a C₁₋₃ alkyl-5-11 membered heteroaryl or C₁₋₃ alkyl-5-8 membered heteroaryl), or a (cycloalkyl)alkyl (such as C₁₋₃ alkyl-C₃₋₈ cycloalkyl),
   wherein A₁ and A₂ are each optionally substituted with one or more (such as 1, 2, 3, 4, or more) substituents independently selected from halogen, hydroxyl, cyano, nitro, amino, alkyl (such as C₁-C₆ alkyl), alkoxy (such as C₁-C₄ alkoxy or methoxy), cycloalkyl (such as C₃-C₈ cycloalkyl, haloalkyl (such as C₁-C₂haloalkyl), haloalkoxy (such as C₁-C₂haloalkoxy), aryl (such as a C₆ or C₈ aryl), heteroaryl (such as a 5-8 membered heteroaryl), heterocycloalkyl (such as a 3-8 or 5-8 membered heterocycloalkyl), an aryloxy (such as C₆ or C₈ aryloxy or phenoxy), a thioaryl (such as C₆ or C₈ thioaryl or thiophenyl), —CHO, —COOH, —CONR₅R₆, —COR₅, —NO₂, —O—CO—R₆, —NR₅—CO—R₆, and hydrocarbyl having a hydrocarbon chain of 1 to 6 carbon atoms in which the carbon atoms are joined by single, double or triple bonds, and any one carbon atom or any two nonadjacent carbon atoms can be replaced by O, NH, N(C₁-C₄alkyl), or S, wherein the hydrocarbyl group is optionally substituted with one or more (e.g., 1, 2, 3, or 4) substituents independently selected from hydroxyl, oxo, halogen, and amino; and
R₁, R₂, R₃, R₄, R₅, and R₆ are each independently hydrogen, halogen, hydroxyl, cyano, nitro, amino, —CHO, —CONH₂, —COaryl (such as benzoyl), —CO-alkyl (such as acetyl), alkyl (such as C₁-C₆ alkyl), alkoxy (such as C₁-C₄ alkoxy), cycloalkyl (such as C₃-C₅ cycloalkyl), haloalkyl (such as C₁-C₂haloalkyl), haloalkoxy (such as C₁-C₂haloalkoxy), aryl (such as a C₆₋₁₁ aryl, a C₆, C₈ or C₁₁ aryl, phenyl, naphthyl, or tolyl), heteroaryl (such as 5-8 membered heteroaryl, furyl, or benzofuryl), heterocycloalkyl (such as a 3-8 membered heterocycloalkyl), an aryloxy (such as C₆ aryloxy, C₈ aryloxy, or phenoxy), alkylaryl (such as C₁₋₃ alkyl-C₆ or C₈ aryl or —CH₂—CH₂-phenyl), or a thioaryl (such as C₆ thioaryl, C₈ thioaryl, or thiophenyl),
wherein two adjacent R groups, R₁ and R₂, R₂ and R₃, or R₃ and R₄ are optionally combined together with the carbon atoms they are attached to form a 5-6-membered aromatic ring or 5-8 membered heteroaromatic ring;
wherein A₂ is optionally substituted by one substituent having the structure —CONR₅ₐR₆ₐ (such as —CONHR₆ₐ), wherein R₅ₐ is hydrogen or a C₁₋₃ alkyl, and R₆ₐ is a bicyclic heteroaryl (such as a 9-11 or 10 membered bicyclic heteroaryl) or a C₆ or C₈ aryl substituted with a bicyclic heteroaryl (such as a 9-11 or 10 membered bicyclic heteroaryl), and
wherein the method is effective in treating, preventing, or ameliorating the disease or disorder associated with a picornavirus infection, or at least one symptom of the disease or disorder associated with a picornavirus infection.

In any aspect or embodiment described herein, (i) A₁ is an monocyclic aryl (such as a C₆ or C₈ aryl), a monocyclic heteroaryl (such as a 5-8 membered heteroaryl having 1, 2, or 3 heteroatoms independently selected from N, O, and S), a mono cycloalkyl (such as a C₃₋₈ cycloalkyl), or a mono heterocycloalkyl (such as a 5-8 membered heterocycloalkyl having 1, 2, or 3 heteroatoms independently selected from N, O, and S); (ii) A₂ is an alkyl (such as C₁₋₆ alkyl) optionally having a heteroatom (such as NH, O, or S) replacing one of the carbon atoms, alkylene (such as such as a C₂₋₆ alkylene), alkenyl (such as a C₂₋₆ alkenyl, ethenyl, or propenyl), alkynyl (such as a C₂₋₆ alkynyl or acetylenyl), a haloalkyl (such as C₁₋₆haloalkyl), cycloalkyl (such as a C₃₋₈ cycloalkyl group), monocyclic aryl (such as phenyl), —CH₂-thioaryl (such as a C₆ or C₈ thioaryl), bicyclic aryl (such as a monocyclic aryl or bicyclic aryl), monocyclic heteroaryl (such as a 5-8 membered heteroaryl having 1, 2, or 3 heteroatoms independently selected from N, O, and S), aryloxy (such as a C₆ or C₈ aryloxy or phenoxy), thioaryl (such as thiophenyl), a bicyclic heteroaryl (such as a 9-11 membered heteroaryl having 1, 2, 3, 4, or 5 heteroatoms independently selected from N, O, and S), alkoxy (such as C₁₋₆ alkoxy), alkylaryl (such as a C₁₋₃ alkyl-C₆₋₁₁ aryl, C₁₋₃ alkyl-C₆ aryl, C₁₋₃ alkyl-C₈ aryl or C₁₋₃ alkyl-C₁₁ aryl), alkylheteroaryl (such as a C₁₋₃ alkyl-5-8 membered heteroaryl), (cycloalkyl)alkyl (such as C₁₋₃ alkyl-C₃₋₈ cycloalkyl), (heterocycloalkyl)alkyl (such as C₁₋₃ alkyl-3-8 membered cycloalkyl); (iii) R₁, R₂, R₃, and R₄ are each independently hydrogen, halogen (such as Cl, F, I, or Br), C₁₋₆ alkyl (such as C₁₋₃ alkyl or methyl), hydroxyl, cyano, nitro, amino, —CHO, —CONH₂, —CO-aryl (such as benzoyl), —CO—C₁₋₆ alkyl (such as acetyl), C₁₋₄ alkoxy), C₃₋₈ cycloalkyl), haloalkyl (such as C₁-C₂haloalkyl), haloalkoxy (such as C₁-C₂haloalkoxy), aryl (such as a C₆, C₈ of C₁₁ aryl, phenyl, naphthyl, or tolyl), 5-8 membered heteroaryl (such as a 5- or 6-membered heteroaryl), 3-8 membered heterocycloalkyl (such as a 5- or 6-membered heterocycloalkyl), C₆ or C₈ aryloxy (such as phenoxy), C₁₋₃ alkyl-C₆ or C₈ aryl (such as —CH₂—CH₂-phenyl), or a thioaryl (such as thiophenyl); or (iv) a combination thereof, wherein (a) when A₂ is an alkyl, alkylene, alkenyl or alkynyl, (i) R₂ is an alkyl (such as methyl, ethyl, or propyl), (ii) R₂ and R₃ combined together with the carbon atoms they are attached to form a C₆ aryl, C₈ aryl, or 5-8 membered heteroaryl, or (iii) A₂ is substituted by an aryl (such as a C₆ or C₈ aryl), aryloxy (such as a C₆ or C₈ aryloxy), or a thioaryl (such as a C₆ or C₈ thioaryl), each of which are optionally substituted with one or more (such as 1, 2, 3, 4, or more) substituted independently selected from halogen (such as Cl, Br, I, or F), C₁₋₃ alkyl (such as methyl); (b) A₂ is optionally substituted by one —CONR₅R₆ₐ or one or more (such as 1, 2, 3, 4, or more) substituent independently selected from halogen (such as Br or Cl), oxo, hydroxyl, C₁₋₃ alkyl, a C₁₋₃ alkoxy (such as methoxy), —CHO, —COOH, —CONR₅R₆, —COR₅, —NO₂, —O—CO—R₅, —NR₅—CO—R₆, C₃₋₈ membered cycloalkyl, a 3-8 membered heterocycloalkyl having 1, 2 or 3 heteroatoms independently selected from N, O and S, C₆ or C₈ aryl, and 5-8 membered heteroaryl having 1, 2 or 3 heteroatoms independently selected from N, O and S; (c) R₆ₐ is a bicyclic heteroaryl (such as a 9-11 or 10 membered bicyclic heteroaryl) or a C₆ or C₈ aryl substituted with a bicyclic heteroaryl (such as a 9-11 or 10 membered bicyclic heteroaryl); (d) each R₅ is independently H or —CH₃, wherein each cyclic substituent is optionally substituted with one or more (such as 1, 2, 3, 4, or more) substituted independently selected from halogen (such as Cl, Br, I, or F), C₁₋₃ alkyl (such as methyl), and —NO₂; and (e) each R₆ is independently H or —CH₃, wherein each cyclic substituent is optionally substituted with one or more (such as 1, 2, 3, 4, or more) substituted independently selected from halogen (such as Cl, Br, I, or F), C₁₋₃ alkyl (such as methyl), and —NO₂.

In any aspect or embodiment described herein, (i) A₁ is a 6-membered aryl or heteroaryl; (ii) L is CONH; (iii) R₁ and R₄ are hydrogen; (iv) R₂ is a hydrogen or a C₁₋₃ alkyl and R₃ is hydrogen, or R₂ and R₃ are combined together with the carbon atoms they are attached to form a C₆ aryl; (v) X is N; or (vi) a combination thereof.

In any aspect or embodiment described herein, A₁ is a 6-membered aryl or heteroaryl with the chemical structure

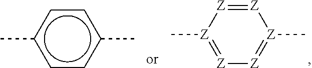

wherein each Z is independently a CH or N, wherein there 0, 1, 2, 3, or 4 (such as 0, 1, 2, or 3) N groups.

In any aspect or embodiment described herein, (i) A₁ is a 6-8 membered monocyclic aromatic group comprising 1, 2, or 3 heteroatoms selected from O, S, and N (e.g., C₆ aryl, C₈ aryl, or a 5-8 membered heteroaryl, such as, 5-7 membered heteroaryl or a 6-membered heteroaryl); (ii) A₂ is a C₁₋₆ alkyl, cycloalkyl (e.g., C₃₋₈ cycloalkyl), C₆₋₁₁ aryl, or 5-8 membered heteroaryl, wherein A₂ is optionally substituted by one or more (such as 1, 2, 3, 4, or more) substituent independently selected from halogen (such as Br or Cl), oxo, hydroxyl, C₁₋₃ alkyl, a C₁₋₃ alkoxy (such as methoxy), —CHO, —COOH, —CONR₅R₆, —COR₅, —NO₂, —O—CO—R₅, —NR₅—CO—R₆, C₃₋₈ membered cycloalkyl, a 3-8 membered heterocycloalkyl having 1, 2 or 3 heteroatoms independently selected from N, O and S, C₆ or C₈ aryl, and 5-8 membered heteroaryl having 1, 2 or 3 heteroatoms independently selected from N, O and S; (iii) L is CONR₅, wherein R₅ is a H or C₁₋₃ alkyl; (iv) R₃ is hydrogen and R₂ is a hydrogen, halogen, or a C₁₋₃ alkyl, or R₂ and R₃ are combined together with the carbon atoms they are attached to form a 6-membered aryl or heteroaryl; (v) X is N or CH; (vi) R₁ and R₄ are each H; or (vii) a combination thereof

In any aspect or embodiment described herein, (i) A₁ is a 6-membered aryl or heteroaryl; (ii) L is CONH; (iii) R₁ and R₄ are hydrogen; (iv) R₂ is a hydrogen or a C₁₋₃ alkyl and R₃ is hydrogen, or R₂ and R₃ are combined together with the carbon atoms they are attached to form a C₆ aryl; (v) X is N; or (vi) a combination thereof

In any aspect or embodiment described herein, the compound is selected from compounds 1-50.

In an aspect, disclosed is a method of treating or preventing a disease, a disorder, or symptom associated with a picornavirus infection in a subject, the method comprising:
providing and administering a therapeutically effective amount of a compound of Formula II, or a pharmaceutically acceptable salt thereof, or a composition comprising the same to the subject, wherein Formula II has the chemical structure:

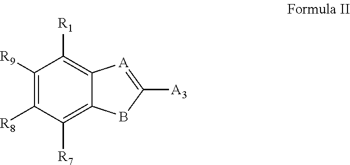

wherein in Formula II,
A is CR₁₁ or N;
B is CR₁₁R₁₂, NR₁₁, O, or S;
A₃ is an aryl (such as a monocyclic aryl or bicyclic aryl), a heteroaryl (such as a monocyclic heteroaryl or bicyclic heteroaryl), an aryloxy (such as phenoxy), a thioaryl (such as thiophenyl), an alkyl (such as C₁-C₆ alkyl), an alkylene (such as a C₂₋₆ alkylene), an alkenyl (such as a C₂₋₆ alkenyl, ethenyl, propenyl), an alkynyl (such as a C₂₋₆ alkynyl or acetylenyl), a haloalkyl (such as C₁-C₆ haloalkyl), an alkoxy (such as C₁-C₆ alkoxy), a cycloalkyl (such as a C₃-C₈ cycloalkyl group), a heterocycloalkyl (such as a 5-8 membered heterocycloalkyl), an alkylaryl (such as a C₁₋₃ alkyl-C₈₋₁₁ aryl, C₁₋₃ alkyl-C₆ aryl or C₁₋₃ alkyl-C₈ aryl or C₁₋₃ alkyl-C₁₁ aryl), an alkylheteroaryl (such as a C₁₋₃ alkyl-5-11 membered heteroaryl or C₁₋₃ alkyl-5-8 membered heteroaryl), or a (cycloalkyl)alkyl (such as C₁₋₃ alkyl-C₃₋₈ cycloalkyl),
   wherein A₃ is optionally substituted with one or more (such as 1, 2, 3, or more) substituents independently selected from halogen (such as Br, F, I, or Cl), hydroxyl, cyano, nitro, amino, alkyl (such as C₁-C₆ alkyl), alkoxy (such as C₁-C₄alkoxy or methoxy), cycloalkyl (such as C₃-C₈ cycloalkyl), haloalkyl (such as C₁-C₂haloalkyl), haloalkoxy (such as C₁-C₂ haloalkoxy), aryl (such as a C₆ or C₈ aryl), heteroaryl (such as a 5-8 membered heteroaryl), heterocycloalkyl (such as a 3-8 or 5-8 membered heterocycloalkyl), an aryloxy (such as C₆ or C₈ aryloxy or phenoxy), a thioaryl (such as C₆ or C₈ thioaryl or thiophenyl), —CHO, —COOH, —CONR₁₁R₁₂, —COR₁₁, —NR₁₁—CO—R₁₂, —NR₁₁ₐCO—R₁₂ₐ (such as —NH—CO—R₁₂ₐ), and hydrocarbyl having a hydrocarbon chain of 1 to 6 carbon atoms in which the carbon atoms are joined by single, double or triple bonds, and any one carbon atom or any two nonadjacent carbon atoms can be replaced by O, NH, N(C₁-C₄alkyl), or S, and wherein the hydrocarbyl group is optionally substituted with one or more substituents independently chosen from hydroxyl, oxo, halogen, and amino;
R₁₁ₐ is a hydrogen or a C₁₋₃ alkyl;
R₁₂ₐ is a monocyclic heteroaryl (such as a 5-8, 5, 6, or 7 membered heteroaryl), a bicyclic heteroaryl (such as a 9-11 or 10 membered bicyclic heteroaryl), a C₆ or C₈ monocyclic aryl, or a bicyclic aryl (such as a C₉₋₁₁ or C₁₀ bicyclic aryl), wherein the R₁₄ₐ is optionally substituted with one or more (such as 1, 2, or 3) substituents independently selected from halogen (such as Cl, Br, I, or F), C₁₋₃ alkyl (such as methyl), and C₁₋₃ alkoxy (such as methoxy);
R₇, R₈, R₉, R₁₀, R₁₁, and R₁₂ are each independently hydrogen, halogen, hydroxyl, cyano, nitro, amino, —CHO, —CONH₂, —NR₁₃—CO—R₁₄, —CO-aryl (such as benzoyl), —CO-heteroaryl (such as 5-8, 5 or 6 membered heteroaryl), —CO-alkyl (such as acetyl), alkyl (such as C₁-C₆ alkyl), alkoxy (such as C₁-C₄alkoxy), cycloalkyl (such as C₃-C₈ cycloalkyl), haloalkyl (such as C₁-C₂ haloalkyl), haloalkoxy (such as C₁-C₂haloalkoxy), aryl (such as C₆ or C₈ aryl, phenyl, naphthyl, or tolyl), heteroaryl (such as 5-11, 5-8, 5, or 6 membered heteroaryl, furyl, or benzofuryl), heterocycloalkyl (such as 5-11, 5-8, 5, or 6 membered heterocycloalkyl), an aryloxy (such as C₆ or C₈ aryloxy or phenoxy), alkylaryl (such as —CH₂—CH₂-phenyl), or a thioaryl (such as C₆ or C₈ thioaryl or thiophenyl);
R₁₃ and R₁₄ are each independently hydrogen, halogen, hydroxyl, cyano, nitro, amino, —CHO, —CONH₂, —CO-aryl (such as —CO—C₆ or C₈ aryl or benzoyl), —CO-heteroaryl (such as 5-8, 5 or 6 membered heteroaryl), —CO-alkyl (such as —CO—C₁₋₃ alkyl or acetyl), alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), haloalkoxy (such as C₁-C₂haloalkoxy), aryl (such as C₅₋₈, C₅, or C₆ aryl, phenyl, naphthyl, or tolyl), heteroaryl (such as 5-8, 5, or 6 membered heteroaryl, furyl, or benzofuryl), heterocycloalkyl (such as 5-8, 5, or 6 membered heterocycloalkyl), an aryloxy (such as C₆ or C₈ aryloxy or phenoxy), alkylaryl (such as C₁₋₃-C₆ or C₈ aryl or —CH₂—CH₂-phenyl), or a thioaryl (such as C₆ or C₈ thioaryl or thiophenyl), wherein R₁₄ is optionally substituted with one or more (such as 1, 2, or 3) substituents independently selected from halogen (such as Cl, Br, I, or F), C₁₋₃ alkyl (such as methyl), and C₁₋₃ alkoxy (such as methoxy);
wherein two adjacent R groups, R₇ and R₈, R₈ and R₉, or R₉ and R₁₀ are optionally combined together with the carbon atoms they are attached to form a 5-6-membered aromatic ring or 5-8 membered heteroaromatic ring;
wherein the method is effective in treating, preventing, or ameliorating the disease or disorder associated with a picornavirus infection, or at least one symptom of the disease or disorder associated with a picornavirus infection.

In any aspect or embodiment described herein, (i) A₃ is a C₆ aryl or 6-membered heteroaryl; (ii) A₃ is optionally substituted with 1 or 2 substituents independently selected from halogen (such as Br, F, I, or Cl), —NR₁₁ₐ—CO—R₁₂ₐ (such as —NH—CO—R₁₂ₐ), hydroxyl, cyano, nitro, amino (such as —NH₂), C₁₋₃ alkyl (such as methyl), C₁₋₄ alkoxy (such as methoxy), C₃₋₈ cycloalkyl, C₁₋₂haloalkyl, C₁₋₂haloalkoxy, C₆ aryl, C₈ aryl, 5-8 membered heteroaryl, 3-8 membered heterocycloalkyl (such as 5-8 membered heterocycloalkyl), —CHO, and —COOH; (iii) R₁₁ₐ is a hydrogen or a C₁₋₃ alkyl; (iv) R₁₂ₐ is a 5-8 membered heteroaryl (such as 5, 6, or 7 membered heteroaryl), 9-11 membered bicyclic heteroaryl (such as 10 membered bicyclic heteroaryl), C₆ monocyclic aryl, C₈ monocyclic aryl, or C₉₋₁₁ bicyclic aryl (such as C₁₀ bicyclic aryl), wherein the R₁₄ₐ is optionally substituted with 1, 2, or 3 substituents independently selected from halogen (such as Cl, Br, I, or F), or C₁₋₃ alkyl (such as methyl), and C₁₋₃ alkoxy (such as methoxy); (v) R₇, R₉, and R₁₀ are each hydrogen; (vi) A is N; (vii) B is NR₁₁; (viii) R₁₁ is hydrogen or C₁₋₆ alkyl (such as C₁₋₃ alkyl or methyl); (ix) R₁₃ is a hydrogen or a C₁₋₃ alkyl; (x) R₁₄ is hydrogen, alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), aryl (such as phenyl, naphthyl, or tolyl), heteroaryl (such as furyl, thienyl, or pyrrolyl), and heterocycloalkyl (such as 5-8, 5, or 6 membered heterocycloalkyl); a combination thereof; or (xi) a combination thereof.

In any aspect or embodiment described herein, A₃ is a C₆ aryl or 6-membered heteroaryl having the chemical structure

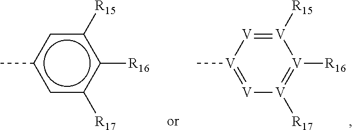

wherein: (i) [symbol] is the point of attachment with Formula II; each V is independently a CH or N, wherein there 0, 1, 2, 3, or 4 (such as 0, 1, 2, or 3) N groups; and (ii) R₁₅, R₁₆, and R₁₇ are independently selected from halogen (such as Br, F, I, or Cl), —NR₁₃ₐCOR₁₄ₐ (such as —NHCOR₁₄ₐ), hydroxyl, cyano, nitro, amino (such as —NH₂), C₁₋₃ alkyl (such as methyl), C₁₋₄alkoxy (such as methoxy), C₃₋₈ cycloalkyl, C₁₋₂ haloalkyl, C₁₋₂ haloalkoxy, C₆ aryl, C₈ aryl, 5-8 membered heteroaryl, 3-8 membered heterocycloalkyl (such as 5-8 membered heterocycloalkyl), —CHO, and —COOH, wherein (a) one of R₁₅, R₁₆, and R₁₇ is a hydrogen; and (b) one of R₁₅, R₁₆, and R₁₇ is a hydrogen or a C₁₋₆ alkyl (such as C₁₋₃ alkyl or methyl).

In any aspect or embodiment described herein, (i) A₃ is an aryl (such as C₆ or C₈ aryl), a heteroaryl (such as 5-8 membered heteroaryl), a cycloalkyl (such as a C₃₋₈ cycloalkyl group), or a heterocycloalkyl (such as a 5-8 membered heterocycloalkyl); (ii) A₃ is optionally substituted with one or more (such as 1 or 2) substituents independently selected from halogen (such as Br, F, I, or Cl), —NR₁₁ₐ—CO—R₁₂ₐ (such as —NH—CO—R₁₂ₐ), hydroxyl, cyano, nitro, amino (such as —NH₂), C₁₋₃ alkyl (such as methyl), C₁₋₄ alkoxy (such as methoxy), C₃₋₈ cycloalkyl, C₁₋₂ haloalkyl, C₁₋₂ haloalkoxy, C₆ or C₈ aryl, 5-8 membered heteroaryl, 3-8 membered heterocycloalkyl (such as 5-8 membered heterocycloalkyl), —CHO, —COOH, and hydrocarbyl having a hydrocarbon chain of 1 to 3 carbon atoms in which the carbon atoms are joined by single, double or triple bonds, and any one carbon atom or any two nonadjacent carbon atoms can be replaced by O, NH, N(C₁-C₄ alkyl), or S, and wherein the hydrocarbyl group is optionally substituted with one or more substituents independently chosen from hydroxyl, oxo, halogen, and amino; (iii) B is CR₁₁R₁₂ or NR₁₁; (iv) R₁₁ and R₁₂ are each independently hydrogen, halogen, hydroxyl, cyano, nitro, amino, —CHO, —CONH₂, C₁₋₃ alkyl (such as methyl), C₁₋₃ alkoxy, C₁₋₂ haloalkyl, and C₁₋₂ haloalkoxy; (v) R₇, R₈, R₉, and R₁₀ are each independently hydrogen, halogen, hydroxyl, cyano, nitro, amino, —CHO, —CONH₂, —NR₁₃—CO—R₁₄, —CO-aryl (such as —CO—C₆ or C₈ aryl or benzoyl), —CO-alkyl (such as —CO—C₁₋₆ alkyl or acetyl), alkyl (such as C₁₋₆ alkyl or methyl), alkoxy (such as C₁₋₄ alkoxy), cycloalkyl (such as C₃₋₈ cycloalkyl), haloalkyl (such as C₁₋₂ haloalkyl), haloalkoxy (such as C₁₋₂ haloalkoxy), aryl (such as a C₆ or C₈ aryl), heteroaryl (such as 5-8, 5, or 6 membered heteroaryl, furyl, thienyl, or pyrrolyl), heterocycloalkyl (such as a 3-8 membered heterocycloalkyl), an aryloxy (such as phenoxy), alkylaryl (such as —CH₂—CH₂-phenyl), or a thioaryl (such as thiophenyl); (vi) R₁₃ and R₁₄ are each independently hydrogen, alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), aryl (such as phenyl, naphthyl, or tolyl), heteroaryl (such as furyl, thienyl, or pyrrolyl), and heterocycloalkyl (such as 5-8, 5, or 6 membered heterocycloalkyl); or (vii) a combination thereof.

In any aspect or embodiment described herein, either (i) or (ii):
(i) A₃ is an aryl (such as a monocyclic aryl or bicyclic aryl), a heteroaryl (such as a monocyclic heteroaryl or bicyclic heteroaryl), a cycloalkyl (such as a C₃₋₈ cycloalkyl group), or a heterocycloalkyl (such as a 5-8 membered heterocycloalkyl), wherein:
   (a) one carbon atom (such as R₁₅, R₁₆, or R₁₇) is —NR₁₁ₐ—CO—R₁₂ₐ (such as —NH—CO—R₁₂ₐ), cycloalkyl (such as C₃-C₅ cycloalkyl), aryl (such as a C₆ or C₈ aryl), heteroaryl (such as a 5-8 membered heteroaryl), heterocycloalkyl (such as a 3-8 or 5-8 membered heterocycloalkyl), —CHO, or —COOH; and
   (b) a second carbon atom (such as R₁₅, R₁₆, or R₁₇) is optionally substituted with a halogen (such as Br, F, I, or Cl), hydroxyl, cyano, nitro, amino (such as —NH₂), alkyl (such as C₁₋₆ or C₁₋₃ alkyl or methyl), alkoxy (such as C₁₋₄ alkoxy or methoxy), haloalkoxy (such as C₁₋₂ haloalkoxy); or
(ii) R₇, R₈, R₉, and R₁₀ are each independently selected from hydrogen, halogen, hydroxyl, cyano, nitro, amino, —CHO, —CONH₂, —CO-alkyl (such as —CO—C₁₋₆ alkyl or acetyl), alkyl (such as C₁₋₆ alkyl or methyl), alkoxy (such as C₁₋₄ alkoxy), cycloalkyl (such as C₃₋₈ cycloalkyl), haloalkyl (such as C₁₋₂ haloalkyl), and haloalkoxy (such as C₁₋₂ haloalkoxy),
   wherein R₈ or R₉ (such as R₈) is —NR₁₃—CO—R₁₄, —CO-aryl (such as —CO—C₆ or C₈ aryl or benzoyl), —CO-heteroaryl (such as 5-8, 5 or 6 membered heteroaryl), aryl (such as a C₆ or C₈ aryl), heteroaryl (such as 5-8, 5, or 6 membered heteroaryl, furyl, thienyl, or pyrrolyl), heterocycloalkyl (such as a 3-8 membered heterocycloalkyl), an aryloxy (such as phenoxy), alkylaryl (such as —CH₂—CH₂-phenyl), and a thioaryl (such as thiophenyl).

In any aspect or embodiment described herein, the compound is selected from compounds 51-67.

In an aspect, disclosed is a method of treating or preventing a disease, a disorder, or symptom associated with picornaviruses in a subject, the method comprising:

providing and administering a therapeutically effective amount of a compound selected from 1-[bis(4-fluorophenyl)methoxy]-3-(5,6-difluoro-1H-1,3-benzodiazol-1-yl)propan-2-ol (compound 108), (4,6-diamino-1,3,5-triazin-2-yl)methyl 3-[5-fluoro-2-(4-fluorophenyl)-1H-indol-3-yl]propanoate (compound 111), 4-(2-{1-[(2-chlorophenyl)methyl]-1H-indol-3-yl}ethenyl)-6-(trifluoromethyl)-1,2-dihydropyrimidin-2-one (compound 139), and N-(3-chlorophenyl)-2-[8-(3-fluorophenoxy)-3-oxo-2H,3H-[1,2,4]triazolo[4,3-a]pyrazin-2-yl]acetamide (compound 167) or a pharmaceutically acceptable salt thereof, or a composition comprising the same to the subject,
wherein the method is effective in treating or ameliorating the disease, disorder associated with picornaviruses, or at least one symptom of the disease or disorder associated with picornaviruses.

In an aspect, disclosed is a composition for treating or preventing a disease, a disorder, or symptom associated with picornavirus in a subject comprising a therapeutically effective amount of a compound of Formula I or a pharmaceutically acceptable salt thereof, and/or Formula II or a pharmaceutically acceptable salt thereof, 1-[bis(4-fluorophenyl)methoxy]-3-(5,6-difluoro-1H-1,3-benzodiazol-1-yl)propan-2-ol (compound 108), (4,6-diamino-1,3,5-triazin-2-yl)methyl 3-[5-fluoro-2-(4-fluorophenyl)-1H-indol-3-yl]propanoate (compound 111), 4-(2-{1-[(2-chlorophenyl)methyl]-1H-indol-3-yl}ethenyl)-6-(trifluoromethyl)-1,2-dihydropyrimidin-2-one (compound 139), and/or N-(3-chlorophenyl)-2-[8-(3-fluorophenoxy)-3-oxo-2H,3H-[1,2,4]triazolo[4,3-a]pyrazin-2-yl]acetamide (compound 167), or a pharmaceutically acceptable salt thereof, as disclosed herein; wherein the composition is effective in treating or ameliorating the disease, disorder associated with picornaviruses, or at least one symptom of the disease or disorder associated with picornaviruses.

These and other aspects of the present invention are described in more detail below.

### BRIEF DESCRIPTION OF THE DRAWINGS

Reference will now be made to the accompanying drawings, which are not necessarily drawn to scale, and wherein:

FIGS. 1A and 1B: 2C:2C binding pocket formed between 2C monomers. (1A) The surface rendition of Chain F and the cartoon rendition of Chain A from the crystal structure of Enterovirus A71 (EV-A71) 2C (PDB:5GRB) are shown. The α6 helix of Chain A is protruding into the adjacent 2C subunit (Chain F). The adenosine triphosphate (ATP) molecule is shown as sticks. (1B) Part of the Chain F that forms the 2C:2C interacting pocket is shown as surface and the terminal six residues of Chain A are shown as stick/cartoon. The box marks the pocket that was used for the in silico screen.

FIGS. 2A, 2B, 2C, and 2D: Compounds tested using the in vitro 2C ATPase inhibition assay. (2A) 77 compounds were screened to evaluate whether they are able to inhibit the ATPase function of 2C using the colorimetric malachite green based assay at a final concentration of 50 μM. Decrease in absorbance at 660 nm indicates inhibition of the ATPase function. The cut-off for determining successful inhibition was kept at 3× standard deviation (3×SD) from the average values obtained for active 2C ATPase treated with dimethyl sulfoxide (DMSO). Four compounds inhibited 2C ATPase—Compound 108 (unfilled box, left panel), Compound 111 (unfilled triangle, left panel), Compound 139 (unfilled circle, left panel), and Compound 167 (unfilled diamond, left panel). (2B, 2C, and 2D) Dose response curves for Compound 108, Compound 111, Compound 139 and Compound 167 were performed to determine the IC₅₀ values. The concentration range is 0.06 μM to 1000 μM. C) Dose response curves to determine the IC₅₀ values for the two positive controls, Guanidinium HCl (GnHCl) and Fluoxetine, were performed. The concentration range for GnHCl was 0.015 mM to 500 mM and 0.06 μM to 1000 μM for Fluoxetine. Graphed mean and SD, n=3.

FIGS. 3A and 3B: Compounds tested for inhibition of cytopathic effects (CPE) caused by EV-A71 in African Green Monkey kidney cells (Vero cells) and cytotoxicity. (3A) Compounds were screened at a single drug concentration of 50 μM. The Cell Titer Glo® Assay from Promega (Madison, Wisconsin, United States of America) was used to read out the cell viability which directly correlates to the inhibition of CPE caused by EV-A71 and thereby the virus. A threshold of 3× standard deviation (3SD) above the average luminescence readout of the infected cells (Cells+DMSO+Virus) was used to identify the anti-viral compounds. (3B) IC₅₀ values for anti-viral activity of Compound 111 and Compound 167 were calculated using dose response curves and the concentration range was 0.5 μM to 50 μM. C) Cytotoxicity in Vero cells was also determined for these two compounds using dose response curves. The dosage range was from 0.125 μM to 1000 μM. D) Dose response curves for the two positive controls, Guanidinium HCl and Dibucaine, were also generated to determine their respective IC₅₀ values. The concentration range for GnHCl was 0.01 mM to 10 mM and that for Dibucaine was 0.5 μM to 50 μM. Colors as used in FIGS. 2A-2D. Graphed mean and SD, n=3.

FIGS. 4A, 4B, 4C, 4D, 4E, and 4F: Structure activity relationship studies. (4A) Comparison of the chemical structures of adenosine triphosphate (ATP), Compound 111 and Compound 167 showing that by including R-groups at the N⁸ position and by avoiding R-groups at the N⁶ and N⁹ positions, we could select analogs that were different from ATP. (4B) Pharmacophore model developed to select analogs from the in-house library. The central ring is either a benzimidazole (if X=N) or benzoxazole (X=O) with either R₁ or R₂ or both R-groups. Further, it can either have A₁ or both A₁ and A₂ aromatic/heterocyclic linked by an amide or ether or ester linker. (4C) 45 analogs of Compound 111 and Compound 167 were tested for CPE inhibition in the cell-based assay at a final concentration of 50 μM. Six compounds showed luminescence reading above the 3SD cutoff. Compound T3 is shown as unfilled circle for better visualization. (4D) Dose response curve for Compound T3 was carried out to determine its IC₅₀. The dose range for this assay was 1 μM to 200 μM. (4E) CC₅₀ of Compound T3 was also determined in Vero cells. The dose range for this assay was 35 μM to 200 μM. (4F) The chemical structure of Compound T3.

FIGS. 5A, 5B, and 5C. SAR screening of the 67 analogues of the hit Compound T3 using the cell-based assay and the in vitro ATPase assay. (5A) The 67 analogs were tested in the cell-based CPE inhibition assay at 25 μM. Promega Cell Titer Glo® Assay was used for this assay and higher luminescence indicates higher inhibition of the CPE caused by the virus. (5B) Δ2C¹¹⁶⁻³²⁹ ATPase inhibition assay was carried out to evaluate whether these compounds are able to inhibit the ATPase function of 2C using the colorimetric malachite green based assay at a final compound concentration of 50 μM. The stars (and unfilled box) highlight the compounds that showed activity in both the assays. (5C) Dose response curves for Compound 36 (unfilled box from FIG. 5B) were generated to calculate its IC₅₀ value for antiviral activity (CPE inhibition) and Δ2C¹¹⁶⁻³²⁹ ATPase inhibition activity, and the CC₅₀ value for the cytotoxicity. Graphed mean and SD, n=3.

FIG. 6: Differential Scanning Fluorimetry assay. Normalized relative fluorescence units (RFU) is plotted against the Temperature (° C.) to give the melting profile of recombinant EV-A71 2C in presence of increasing concentrations of the Compound 36 (upper panel) and the change in melting temperature when compared to the DMSO treated control (lower panel).

FIG. 7: Validation of resistant escape mutant. A comparison of the viral titers for the DMSO treated vs the drug-treated, and the resulting fold change is shown as a bar graph. The wild type (WT) virus and the T237N mutant were generated from RNA as described previously. The final concentration of Compound 167 used is 500 μM which is slightly less than 10× its IC₅₀ (59.4 μM). Graphed mean and SD, n=3.

FIG. 8: Effect of Compound 36 on viral RNA replication using the EVA71 replicon system. Viral RNA levels are quantified upon transfecting Hela Cells with the RNA generated from the EV-A71 replicon in the presence of either DMSO (vehicle control) or three different concentrations (25 μM, 5 μM and 1 μM) of Compound 36. In this replicon, the P1 capsid encoding region is replaced with the sequence encoding for a fluorescent reporter protein. After 14 hours post transfection, the fluorescence from the reporter protein was read. The untreated GNN (replication defective) and no transfection controls were used to remove the background fluorescence and the data was normalized to the untreated WT values. Graphed mean and SEM, n=3 in duplicate.

FIGS. 9A, 9B, 9C, 9D, 9E, and 9F: Molecular docking of Compound 167 and Compound 36 in the 2C:2C binding pocket and identification of interacting residues. The two compounds, Compound 167 and Compound 36 (stick diagram), were docked into the binding pocket using the Glide docking application. The Induced Fit docking application was used to generate 64 poses for Compound 167 and 51 poses for Compound 43. FIGS. 9A and 9D show the top pose for the two ligands, Compound 167 and Compound 36, respectively in the pocket as a surface view. FIGS. 9B and 9E show the interacting residues in the pocket. FIGS. 9C and 9F show the common residues that interact with the compounds when analyzing the top 10 poses using the Interaction Fingerprint application. The yellow dotted lines show the predicted pi-pi, pi-cation and hydrogen bonds. All the applications are part of the Maestro suite from Schrödinger.

FIGS. 10A, 10B, and 10C: Broad spectrum activity of Compound 36 against EV-D68 and Poliovirus Mahoney. A dose response cure was generated to determine the IC₅₀ for the virus induced CPE inhibition for Compound 36 against EV-D68 (10A) and Poliovirus Mahoney strain (10B) and coxsackievirus-B3 (CV-B3) (10C). The assay was carried out in Vero cells for EV-D68 and Poliovirus and HeLa cells for CV-B3. Graphed mean and SD, n=3.

FIGS. 11A, 11B, and 11C: Multiple 2C protein sequence alignment. (11A) 2C protein sequences from EV-A71 BrCr strain, EV-D68 Fermon strain, Poliovirus-1 Mahoney strain and Coxsackievirus B3 Nancy strain were aligned using the Praline protein alignment tool and the results for conservation of residues is shown. The structural and functional features including Walker A, Walker B, Motif C, Arginine finger and Zinc binding sites are shown using black boxes. (11B) The sequences were also aligned using Clustal Omega and the percent identity matrix was generated and is shown. (11C) Zoomed in portion of the alignment of FIG. 11A). The 2C:2C binding-pocket-forming residues are highlighted with black arrows.

FIG. 12: Schematic of the ATPase assay. 2C is an ATPase and hydrolyzes ATP into ADP and free orthophosphate. This orthophosphate then reacts with the molybdate to form the phosphomolybdate. This complex then reacts with malachite green resulting in a green colored complex that can be quantified by determining the absorbance at 660 nm. This absorbance reading is a direct correlation to the amount of free phosphate produced as a result of the enzymatic ATPase activity of 2C. Inhibition of 2C by the drug will result in decreased amount of orthophosphate that will eventually result in a reduced absorbance reading.

FIGS. 13A and 13B: Compounds tested for inhibition of cytopathic effects (CPE) caused by EV-A71 in Vero cells. Compounds were screened at a single drug concentration of 50 μM. An assay was performed in duplicate (13A, Repeat 1; 13B, Repeat 2) to analyze the effect of drugs on cell viability as well as the cytopathic effect of the virus. The Cell Titer Glo® Assay from Promega was used to read out the cell viability which directly correlates to the inhibition of CPE caused by EV-A71 and thereby the virus. A threshold of 3× standard deviation (3SD) above the average relative luminescence units (RLU) readout of the infected cells (Cells+DMSO+Virus) was used to identify the anti-viral compounds. Compound 108 is shown as an unfilled box, Compound 111 is shown as an unfilled triangle, Compound 139 is shown as an unfilled circle, and Compound 167 is show as an unfilled diamond. The reaction volume was 100 μL.

FIGS. 14A, 14B, 14C, 14D, and 14E: Dose response curves for the other 5 compounds from the structure activity relationship study of the 45 analogs of Compound 111 and Compound 167. Dose-response curves for Compound T1 (14A, Compound T2 (14B), Compound T4 (14C), Compound T5 (14D), and Compound T6 (14E) were also performed to determine their IC₅₀ values. The dose range for this assay was 1 μM to 200 μM. Further, a cytotoxicity dose-response curve for each of the compounds were used to calculate their CC₅₀ values. The dose range for this assay was 35 μM to 200 μM. Graphed mean and SD, n=3.

FIG. 15: 2C ATPase inhibition by Compound 28. Dose-response curve to determine the IC₅₀ of Δ2C¹¹⁶⁻³²⁹ ATPase inhibition for Compound 28 is shown. The concentration of the compound tested ranged from 0.1 μM to 500 μM. Graphed mean and SD, n=3.

FIG. 16: Effect of Compound 36 on viral RNA replication using the EVA71 replicon system. Viral RNA levels are quantified upon transfecting Hela Cells with the RNA generated from the EV-A71 replicon in the presence of either DMSO (vehicle control) or three different concentrations (25 μM, 5 μM and 1 μM) of five compounds from this family of compounds, Compound 20, Compound 28, Compound 36, Compound 43, and Compound 48. In this replicon, the P1 capsid encoding region is replaced with the sequence encoding for a fluorescent reporter protein. After 14 hours post transfection, the fluorescence from the reporter protein was read. The untreated GNN (replication defective) and no transfection controls were used to remove the background fluorescence and the data was normalized to the untreated WT values. Graphed mean and SEM, n=3 in duplicate.

FIG. 17: Proximity of resistant mutant to Compound 36 to encapsidation defect rescue mutant. Left: EV-A71 protomer showing the four capsid proteins—VP1 in yellow, VP2 in cyan, VP3 in magenta and VP4 in red (all are rendered as cartoon) (PDB: 3VBS). Right: A zoomed-in image showing the proximity of the K47 residue in VP4 to the K41 residue in VP3. K47E is the resistant mutation that was observed in this study to Compound 36 while K41R in Poliovirus VP3 is the mutation Wimmer and colleagues observed that rescued an encapsidation defect caused by the mutations K279A/R280A in Poliovirus 2C. It is important to note that the residue in EV-A71 VP3 is already an Arginine.

FIG. 18: Alignment of VP1 sequences from EV-A71, EV-D68, Poliovirus and coxsackievirus-B3 (CV-B3). The box highlights the T237 residue in VP1 in EV-A71 and the equivalent residues in the other viruses. Clustal Omega—Multiple Sequence Alignment tool was used for sequence alignment.

FIG. 19: Validation of resistant escape mutant. Plaque assay for viral titer determination for EV-71 B strain WT (top panel) and VP1 T237N (bottom panel) was carried out using Vero cells. Representative plaque assay plates are shown here. Three dilutions of WT virus and one dilution for the mutant virus when treated with either DMSO (vehicle control) or 500 μM of Compound 167 (equal to 10×IC₅₀) are shown. After 3 days, plaques were developed, and the viral titer was determined to test the susceptibility of the viruses.

### DETAILED DESCRIPTION OF THE INVENTION

The enterovirus (EV) genus includes a number of important human and animal pathogens. Enterovirus A71 (EV-A71), enterovirus D68 (EV-D68), poliovirus (PV), and coxsackievirus (CV) outbreaks have affected millions worldwide causing a range of upper respiratory, skin, neuromuscular diseases, including acute flaccid myelitis, and hand-foot-and-mouth disease. There are no FDA-approved anti-viral therapeutics for these enteroviruses. Disclosed herein is a novel broad spectrum anti-viral compounds targeting the conserved non-structural viral protein 2C that have low micro-molar to nanomolar IC₅₀ values. The selection of resistant mutants resulted in amino acid substitutions in the viral capsid protein, implying a role for 2C in capsid assembly, as has been seen in PV. The inhibitors reported here could be useful probes in understanding the assembly and encapsidation stages of the viral life cycle.

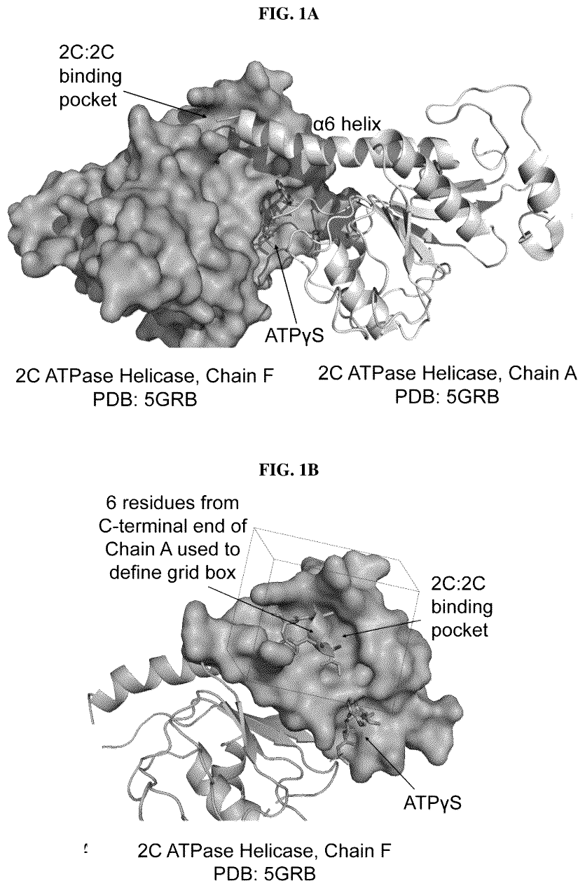

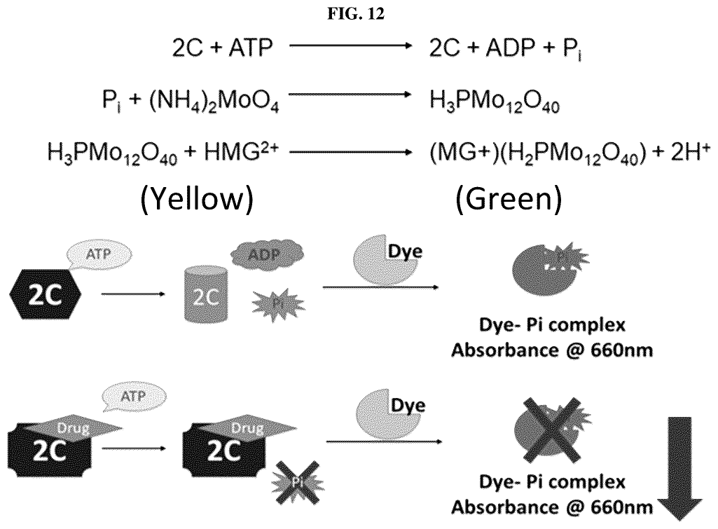

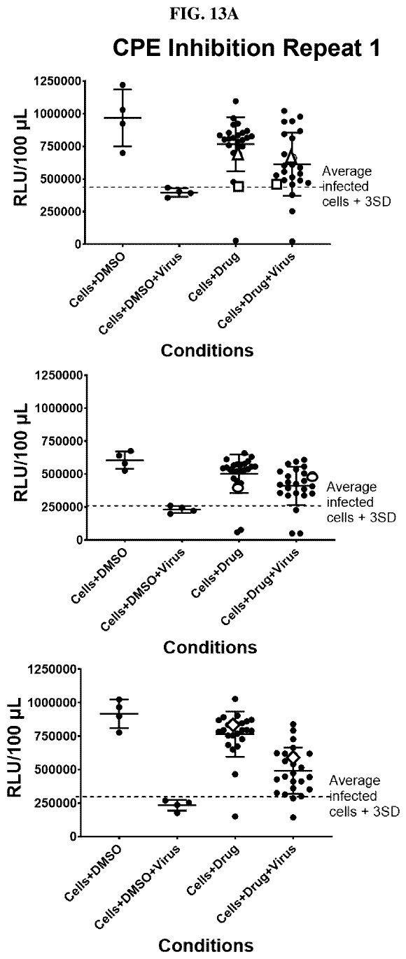

The EV 2C protein is an excellent target for the development of broad-spectrum anti-virals. 2C is indispensable for the virus due to its multifunctional role in the viral life cycle including viral RNA genome replication, encapsidation of nascent RNA strands into new viral particles, rearrangement of cellular membranes, and facilitating the formation of capsid conformations required for efficient uncoating. It is a highly conserved protein among enteroviruses and shows very limited homology to host proteins (FIG. 12A). 2C has ATPase activity when oligomerized into a hexamer and can function in vitro as a AAA+ helicase. Published crystal structures of an N-terminal truncated 2C lacking the first 115 amino acids of the 329-long protein from EV-A71 and poliovirus show a conserved “pocket”. This 2C pocket is adjacent to the adenosine triphosphate (ATP)-binding site and the site of interaction between the C-terminal alpha helix of one 2C subunit and another 2C subunit in the hexameric assembly. (FIG. 1A). The conservation of this pocket across EV 2C proteins makes this an attractive target for therapeutic development (FIG. 12B).

A structure-based drug design approach was used to develop broad-spectrum anti-viral compounds that target the 2C pocket (FIG. 1B). In silico screening of millions of commercially available small molecule compounds was performed to identify those that were predicted to interact with the 2C pocket using AtomNet technology which is a structure-based deep convolutional neural network virtual screening technology. These small molecules were tested in vitro for 2C ATPase inhibition and in cells for anti-viral activity. Using these primary screening approaches and an extensive structure-activity relationship study, a potent compound, Compound 36 with broad-spectrum anti-viral activity against EV-A71, EV-D68 and Poliovirus has been identified. Further, the data suggests that Compound 36 inhibits the encapsidation and assembly of the progeny viruses.

In an aspect, disclosed is a method of treating or preventing a disease, a disorder, or symptom associated with picornaviruses in a subject, the method comprising:
providing and administering a therapeutically effective amount of a compound of Formula I, or a pharmaceutically acceptable salt thereof, or a composition comprising the same to the subject,

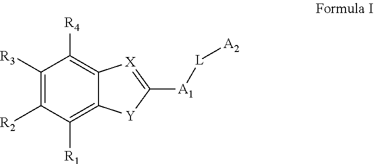

wherein in Formula I,
X is CR₅ or N;
Y is CR₅R₆, NR₅, O, or S;
L is a linker comprising O, NR₅, CONR₅, COO, COS, or S;
A₁ and A₂ are each independently an aryl (such as a mono- or bicyclic aryl group), a heteroaryl (such as a mono- or bicyclic heteroaryl group), an aryloxy (such as phenoxy), a thioaryl (such as thiophenyl), an alkyl (such as C₁-C₆alkyl), an alkylene, an alkenyl (such as ethenyl and propenyl), an alkynyl (such as acetylenyl), a haloalkyl (such as C₁-C₆haloalkyl), an alkoxy (such as C₁-C₆alkoxy), a cycloalkyl (such as a C₃-C₈Cycloalkyl group), an alkylaryl, an alkylheteroaryl, or a “(cycloalkyl)alkyl; which A₁ and A₂ are each optionally substituted with one or more substituents independently chosen from halogen, hydroxyl, cyano, nitro, amino, alkyl such as C₁-C₆alkyl, alkoxy such as C₁-C₄alkoxy, cycloalkyl such as C₃-C₈Cycloalkyl, haloalkyl such as C₁-C₂haloalkyl, haloalkoxy such as C₁-C₂haloalkoxy, aryl, heteroaryl, heterocycloalkyl, an aryloxy such as phenoxy, a thioaryl such as thiophenyl, —CHO, —COOH, —CONR₅R₆, —COR₅, —NR₅—CO—R₆, and hydrocarbyl such as C₁-C₆hydrocarbyl, having a hydrocarbon chain of 1 to 6 carbon atoms in which the carbon atoms are joined by single, double or triple bonds, and any one carbon atom or any two nonadjacent carbon atoms can be replaced by O, NH, N(C₁-C₄alkyl), or S, and which hydrocarbyl group is optionally substituted with one or more substituents independently chosen from hydroxyl, oxo, halogen, and amino; and
R₁, R₂, R₃, R₄, R₅, and R₆ are each independently hydrogen, halogen, hydroxyl, cyano, nitro, amino, —CHO, —CONH₂, -Coaryl such as benzoyl, -Coalkyl such as acetyl, alkyl such as C₁-C₆alkyl, alkoxy such as C₁-C₄alkoxy, cycloalkyl such as C₃-C₈Cycloalkyl, haloalkyl such as C₁-C₂haloalkyl, haloalkoxy such as C₁-C₂haloalkoxy, aryl such as phenyl, naphthyl, or tolyl, heteroaryl such as furyl, benzofuryl, heterocycloalkyl, an aryloxy such as phenoxy, alkylaryl such as —CH₂—CH₂-Ph, or a thioaryl such as thiophenyl;
wherein two adjacent R groups, R₁ and R₂, R₂ and R₃, or R₃ and R₄ are optionally combined together to form a 5-8 membered aromatic or heteroaromatic ring;
wherein the method is effective in treating or ameliorating the disease, disorder associated with picornaviruses, or at least one symptom of the disease or disorder associated with picornaviruses.

For example, an aspect of the present disclosure relates to a method of treating or preventing a disease or disorder, or a symptom associated with picornavirus in a subject, the method comprising:
providing and administering a therapeutically effective amount of a compound of Formula I, or a pharmaceutically acceptable salt thereof, or a composition comprising the same to the subject, wherein Formula I has the chemical structure:

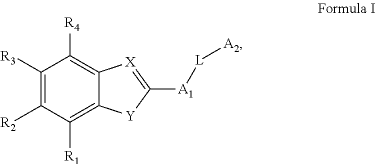

wherein in Formula I,
X is CR₅ or N;
Y is CR₅R₆, NR₅, O, or S;
L is a linker comprising O, NR₅, CONR₅, COO, COS, or S;
A₁ and A₂ are each independently an aryl (such as a monocyclic aryl or bicyclic aryl), a heteroaryl (such as a monoheteroaryl or bicyclic heteroaryl), an aryloxy (such as phenoxy), a thioaryl (such as thiophenyl), an alkyl (such as C₁-C₆ alkyl), an alkylene (such as a C₂₋₆ alkylene), an alkenyl (such as a C₂₋₆ alkenyl, ethenyl, or propenyl), an alkynyl (such as a C₂₋₆ alkynyl or acetylenyl), a haloalkyl (such as C₁-C₆ haloalkyl), an alkoxy (such as C₁-C₆ alkoxy), a cycloalkyl (such as a C₃-C₈ cycloalkyl group), a heterocycloalkyl (such as a 5-8 membered heterocycloalkyl), an alkylaryl (such as a C₁₋₃ alkyl-C₈₋₁₁ aryl, C₁₋₃ alkyl-C₆ aryl, C₁₋₃ alkyl-C₈ aryl, or C₁₋₃ alkyl-C₁₁ aryl), an alkylheteroaryl (such as a C₁₋₃ alkyl-5-11 membered heteroaryl or C₁₋₃ alkyl-5-8 membered heteroaryl), or a (cycloalkyl)alkyl (such as C₁₋₃ alkyl-C₃₋₈ cycloalkyl),
   wherein A₁ and A₂ are each optionally substituted with one or more (such as 1, 2, 3, 4, or more) substituents independently selected from halogen, hydroxyl, cyano, nitro, amino, alkyl (such as C₁-C₆ alkyl), alkoxy (such as C₁-C₄ alkoxy or methoxy), cycloalkyl (such as C₃-C₈ cycloalkyl, haloalkyl (such as C₁-C₂haloalkyl), haloalkoxy (such as C₁-C₂haloalkoxy), aryl (such as a C₆ or C₈ aryl), heteroaryl (such as a 5-8 membered heteroaryl), heterocycloalkyl (such as a 3-8 or 5-8 membered heterocycloalkyl), an aryloxy (such as C₆ or C₈ aryloxy or phenoxy), a thioaryl (such as C₆ or C₈ thioaryl or thiophenyl), —CHO, —COOH, —CONR₅R₆, —COR₅, —NO₂, —O—CO—R₆, —NR₅—CO—R₆, and hydrocarbyl having a hydrocarbon chain of 1 to 6 carbon atoms in which the carbon atoms are joined by single, double or triple bonds, and any one carbon atom or any two nonadjacent carbon atoms can be replaced by O, NH, N(C₁-C₄alkyl), or S, wherein the hydrocarbyl group is optionally substituted with one or more (e.g., 1, 2, 3, or 4) substituents independently selected from hydroxyl, oxo, halogen, and amino; and
R₁, R₂, R₃, R₄, R₅, and R₆ are each independently hydrogen, halogen, hydroxyl, cyano, nitro, amino, —CHO, —CONH₂, —COaryl (such as benzoyl), —CO-alkyl (such as acetyl), alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), haloalkoxy (such as C₁-C₂haloalkoxy), aryl (such as a C₆₋₁₁ aryl, a C₆, C₈ or C₁₁ aryl, phenyl, naphthyl, or tolyl), heteroaryl (such as 5-8 membered heteroaryl, furyl, or benzofuryl), heterocycloalkyl (such as a 3-8 membered heterocycloalkyl), an aryloxy (such as C₆ aryloxy, C₈ aryloxy, or phenoxy), alkylaryl (such as C₁₋₃ alkyl-C₆ or C₈ aryl or —CH₂—CH₂-phenyl), or a thioaryl (such as C₆ thioaryl, C₈ thioaryl, or thiophenyl),
wherein two adjacent R groups, R₁ and R₂, R₂ and R₃, or R₃ and R₄ are optionally combined together with the carbon atoms they are attached to form a 5-6-membered aromatic ring or 5-8 membered heteroaromatic ring;
wherein A₂ is optionally substituted by one substituent having the structure —CONR₅ₐR₆ₐ (such as —CONHR₆ₐ), wherein R₅ₐ is hydrogen or a C₁₋₃ alkyl, and R₆ₐ is a bicyclic heteroaryl (such as a 9-11 or 10 membered bicyclic heteroaryl) or a C₆ or C₈ aryl substituted with a bicyclic heteroaryl (such as a 9-11 or 10 membered bicyclic heteroaryl), and
wherein the method is effective in treating, preventing, or ameliorating the disease or disorder associated with a picornavirus infection, or at least one symptom of the disease or disorder associated with a picornavirus infection.

In any aspect or embodiment described herein, (i) A₁ is an monocyclic aryl (such as a C₆ or C₈ aryl), a monocyclic heteroaryl (such as a 5-8 membered heteroaryl having 1, 2, or 3 heteroatoms independently selected from N, O, and S), a mono cycloalkyl (such as a C₃₋₈ cycloalkyl), or a mono heterocycloalkyl (such as a 5-8 membered heterocycloalkyl having 1, 2, or 3 heteroatoms independently selected from N, O, and S); (ii) A₂ is an alkyl (such as C₁₋₆ alkyl) optionally having a heteroatom (such as NH, O, or S) replacing one of the carbon atoms, alkylene (such as such as a C₂₋₆ alkylene), alkenyl (such as a C₂₋₆ alkenyl, ethenyl, or propenyl), alkynyl (such as a C₂₋₆ alkynyl or acetylenyl), a haloalkyl (such as C₁₋₆haloalkyl), cycloalkyl (such as a C₃₋₈ cycloalkyl group), monocyclic aryl (such as phenyl), —CH₂-thioaryl (such as a C₆ or C₈ thioaryl), bicyclic aryl (such as a monocyclic aryl or bicyclic aryl), monocyclic heteroaryl (such as a 5-8 membered heteroaryl having 1, 2, or 3 heteroatoms independently selected from N, O, and S), aryloxy (such as a C₆ or C₈ aryloxy or phenoxy), thioaryl (such as thiophenyl), a bicyclic heteroaryl (such as a 9-11 membered heteroaryl having 1, 2, 3, 4, or 5 heteroatoms independently selected from N, O, and S), alkoxy (such as C₁₋₆ alkoxy), alkylaryl (such as a C₁₋₃ alkyl-C₆₋₁₁ aryl, C₁₋₃ alkyl-C₆ aryl, C₁₋₃ alkyl-C₈ aryl or C₁₋₃ alkyl-C₁₁ aryl), alkylheteroaryl (such as a C₁₋₃ alkyl-5-8 membered heteroaryl), (cycloalkyl)alkyl (such as C₁₋₃ alkyl-C₃₋₈ cycloalkyl), (heterocycloalkyl)alkyl (such as C₁₋₃ alkyl-3-8 membered cycloalkyl); (iii) R₁, R₂, R₃, and R₄ are each independently hydrogen, halogen (such as Cl, F, or Br), C₁₋₆ alkyl (such as C₁₋₃ alkyl or methyl), hydroxyl, cyano, nitro, amino, —CHO, —CONH₂, —CO-aryl (such as benzoyl), —CO—C₁₋₆ alkyl (such as acetyl), C₁₋₄ alkoxy), C₃₋₈ cycloalkyl), haloalkyl (such as C₁-C₂haloalkyl), haloalkoxy (such as C₁-C₂haloalkoxy), aryl (such as a C₆, C₈ of C₁₁ aryl, phenyl, naphthyl, or tolyl), 5-8 membered heteroaryl (such as a 5- or 6-membered heteroaryl), 3-8 membered heterocycloalkyl (such as a 5- or 6-membered heterocycloalkyl), C₆ or C₈ aryloxy (such as phenoxy), C₁₋₃ alkyl-C₆ or C₈ aryl (such as —CH₂—CH₂-phenyl), or a thioaryl (such as thiophenyl); or (iv) a combination thereof, wherein (a) when A₂ is an alkyl, alkylene, alkenyl or alkynyl, (i) R₂ is an alkyl (such as methyl, ethyl, or propyl), (ii) R₂ and R₃ combined together with the carbon atoms they are attached to form a C₆ aryl, C₈ aryl, or 5-8 membered heteroaryl, or (iii) A₂ is substituted by an aryl (such as a C₆ or C₈ aryl), aryloxy (such as a C₆ or C₈ aryloxy), or a thioaryl (such as a C₆ or C₈ thioaryl), each of which are optionally substituted with one or more (such as 1, 2, 3, 4, or more) substituted independently selected from halogen (such as Cl, Br, I, or F), C₁₋₃ alkyl (such as methyl); (b) A₂ is optionally substituted by one —CONR₅R₆ₐ or one or more (such as 1, 2, 3, 4, or more) substituent independently selected from halogen (such as Br or Cl), oxo, hydroxyl, C₁₋₃ alkyl, a C₁₋₃ alkoxy (such as methoxy), —CHO, —COOH, —CONR₅R₆, —COR₅, —NO₂, —O—CO—R₅, —NR₅—CO—R₆, C₃₋₈ membered cycloalkyl, a 3-8 membered heterocycloalkyl having 1, 2 or 3 heteroatoms independently selected from N, O and S, C₆ or C₈ aryl, and 5-8 membered heteroaryl having 1, 2 or 3 heteroatoms independently selected from N, O and S; (c) R₆ₐ is a bicyclic heteroaryl (such as a 9-11 or 10 membered bicyclic heteroaryl) or a C₆ or C₈ aryl substituted with a bicyclic heteroaryl (such as a 9-11 or 10 membered bicyclic heteroaryl); (d) each R₅ is independently H or —CH₃, wherein each cyclic substituent is optionally substituted with one or more (such as 1, 2, 3, 4, or more) substituted independently selected from halogen (such as Cl, Br, I, or F), C₁₋₃ alkyl (such as methyl), and —NO₂; and (e) each R₆ is independently H or —CH₃, wherein each cyclic substituent is optionally substituted with one or more (such as 1, 2, 3, 4, or more) substituted independently selected from halogen (such as Cl, Br, I, or F), C₁₋₃ alkyl (such as methyl), and —NO₂.

In any aspect or embodiment described herein, (i) A₁ is a 6-membered aryl or heteroaryl; (ii) L is CONH; (iii) R₁ and R₄ are hydrogen; (iv) R₂ is a hydrogen or a C₁₋₃ alkyl and R₃ is hydrogen, or R₂ and R₃ are combined together with the carbon atoms they are attached to form a C₆ aryl; (v) X is N; or (vi) a combination thereof.

In any aspect or embodiment described herein, A₁ is a 6-membered aryl or heteroaryl with the chemical structure

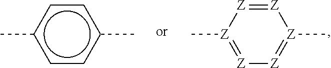

wherein each Z is independently a CH or N, wherein there 0, 1, 2, 3, or 4 (such as 0, 1, 2, or 3) N groups.

In any aspect or embodiment described herein, (i) A₁ is a 6-8 membered monocyclic aromatic group comprising 1, 2, or 3 heteroatoms selected from O, S, and N (e.g., C₆ aryl, C₈ aryl, or a 5-8 membered heteroaryl, such as, 5-7 membered heteroaryl or a 6-membered heteroaryl); (ii) A₂ is a C₁₋₆ alkyl, cycloalkyl (e.g., C₃₋₈ cycloalkyl), C₆₋₁₁ aryl, or 5-8 membered heteroaryl, wherein A₂ is optionally substituted by one or more (such as 1, 2, 3, 4, or more) substituent independently selected from halogen (such as Br or Cl), oxo, hydroxyl, C₁₋₃ alkyl, a C₁₋₃ alkoxy (such as methoxy), —CHO, —COOH, —CONR₅R₆, —COR₅, —NO₂, —O—CO—R₅, —NR₅—CO—R₆, C₃₋₈ membered cycloalkyl, a 3-8 membered heterocycloalkyl having 1, 2 or 3 heteroatoms independently selected from N, O and S, C₆ or C₈ aryl, and 5-8 membered heteroaryl having 1, 2 or 3 heteroatoms independently selected from N, O and S; (iii) L is CONR₅, wherein R₅ is a H or C₁₋₃ alkyl; (iv) R₃ is hydrogen and R₂ is a hydrogen, halogen, or a C₁₋₃ alkyl, or R₂ and R₃ are combined together with the carbon atoms they are attached to form a 6-membered aryl or heteroaryl; (v) X is N or CH; (vi) R₁ and R₄ are each H; or (vii) a combination thereof

In any aspect or embodiment described herein, (i) A₁ is a 6-membered aryl or heteroaryl; (ii) L is CONH; (iii) R₁ and R₄ are hydrogen; (iv) R₂ is a hydrogen or a C₁₋₃ alkyl and R₃ is hydrogen, or R₂ and R₃ are combined together with the carbon atoms they are attached to form a C₆ aryl; (v) X is N; or (vi) a combination thereof

In any aspect or embodiment described herein, the compound is selected from compounds 1-50.

In an aspect, disclosed is a method of treating or preventing a disease, a disorder, or symptom associated with picornaviruses in a subject, the method comprising:
providing and administering a therapeutically effective amount of a compound of Formula II, or a pharmaceutically acceptable salt thereof, or a composition comprising the same to the subject,

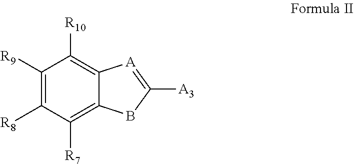

wherein in Formula II,
A is CR₁₁ or N;
B is CR₁₁R₁₂, NR₁₁, O, or S;
A₃ is an aryl (such as a mono- or bicyclic aryl group), a heteroaryl (such as a mono- or bicyclic heteroaryl group), an aryloxy (such as phenoxy), a thioaryl (such as thiophenyl), an alkyl (such as C₁-C₆alkyl), an alkylene, an alkenyl (such as ethenyl and propenyl), an alkynyl (such as acetylenyl), a haloalkyl (such as C₁-C₆haloalkyl), an alkoxy (such as C₁-C₆alkoxy), a cycloalkyl (such as a C₃-C₅Cycloalkyl group), an alkylaryl, an alkylheteroaryl, or a “(cycloalkyl)alkyl; which A₁ is optionally substituted with one or more substituents independently chosen from halogen, hydroxyl, cyano, nitro, amino, alkyl such as C₁-C₆alkyl, alkoxy such as C₁-C₄alkoxy, cycloalkyl such as C₃-C₈Cycloalkyl, haloalkyl such as C₁-C₂haloalkyl, haloalkoxy such as C₁-C₂haloalkoxy, aryl, heteroaryl, heterocycloalkyl, an aryloxy such as phenoxy, a thioaryl such as thiophenyl, —CHO, —COOH, —CONR₁₁R₁₂, —COR₁₁, —NR₁₁—CO—R₁₂, and hydrocarbyl such as C₁-C₆hydrocarbyl, having a hydrocarbon chain of 1 to 6 carbon atoms in which the carbon atoms are joined by single, double or triple bonds, and any one carbon atom or any two nonadjacent carbon atoms can be replaced by O, NH, N(C₁-C₄alkyl), or S, and which hydrocarbyl group is optionally substituted with one or more substituents independently chosen from hydroxyl, oxo, halogen, and amino; and
R₇, R₈, R₉, R₁₀, R₁₁, and R₁₂ are each independently hydrogen, halogen, hydroxyl, cyano, nitro, amino, —CHO, —CONH₂, —NR₁₃—CO—R₁₄, -Coaryl such as benzoyl, -Coalkyl such as acetyl, alkyl such as C₁-C₆alkyl, alkoxy such as C₁-C₄alkoxy, cycloalkyl such as C₃-C₈Cycloalkyl, haloalkyl such as C₁-C₂haloalkyl, haloalkoxy such as C₁-C₂haloalkoxy, aryl such as phenyl, naphthyl, or tolyl, heteroaryl such as furyl, benzofuryl, heterocycloalkyl, an aryloxy such as phenoxy, alkylaryl such as —CH₂—CH₂-Ph, or a thioaryl such as thiophenyl;
R₁₃ and R₁₄ are each independently hydrogen, halogen, hydroxyl, cyano, nitro, amino, —CHO, —CONH₂, -Coaryl such as benzoyl, -Coalkyl such as acetyl, alkyl such as C₁-C₆alkyl, alkoxy such as C₁-C₄alkoxy, cycloalkyl such as C₃-C₈Cycloalkyl, haloalkyl such as C₁-C₂haloalkyl, haloalkoxy such as C₁-C₂haloalkoxy, aryl such as phenyl, naphthyl, or tolyl, heteroaryl such as furyl, benzofuryl, heterocycloalkyl, an aryloxy such as phenoxy, alkylaryl such as —CH₂—CH₂-Ph, or a thioaryl such as thiophenyl;
wherein two adjacent R groups, R₇ and R₈, R₈ and R₉, or R₉ and R₁₀ are optionally combined together to form a 5-8 membered aromatic or heteroaromatic ring;
wherein the method is effective in treating or ameliorating the disease, disorder associated with picornaviruses, or at least one symptom of the disease or disorder associated with picornaviruses.

For example, an aspect of the present disclosure provides a method of treating or preventing a disease, a disorder, or symptom associated with a picornavirus infection in a subject, the method comprising:
providing and administering a therapeutically effective amount of a compound of Formula II, or a pharmaceutically acceptable salt thereof, or a composition comprising the same to the subject, wherein Formula II has the chemical structure:

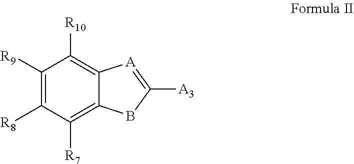

wherein in Formula II,
A is CR₁₁ or N;
B is CR₁₁R₁₂, NR₁₁, O, or S;
A₃ is an aryl (such as a monocyclic aryl or bicyclic aryl), a heteroaryl (such as a monocyclic heteroaryl or bicyclic heteroaryl), an aryloxy (such as phenoxy), a thioaryl (such as thiophenyl), an alkyl (such as C₁-C₆ alkyl), an alkylene (such as a C₂₋₆ alkylene), an alkenyl (such as a C₂₋₆ alkenyl, ethenyl, propenyl), an alkynyl (such as a C₂₋₆ alkynyl or acetylenyl), a haloalkyl (such as C₁-C₆ haloalkyl), an alkoxy (such as C₁-C₆ alkoxy), a cycloalkyl (such as a C₃-C₈ cycloalkyl group), a heterocycloalkyl (such as a 5-8 membered heterocycloalkyl), an alkylaryl (such as a C₁₋₃ alkyl-C₈₋₁₁ aryl, C₁₋₃ alkyl-C₆ aryl or C₁₋₃ alkyl-C₈ aryl or C₁₋₃ alkyl-C₁₁ aryl), an alkylheteroaryl (such as a C₁₋₃ alkyl-5-11 membered heteroaryl or C₁₋₃ alkyl-5-8 membered heteroaryl), or a (cycloalkyl)alkyl (such as C₁₋₃ alkyl-C₃₋₈ cycloalkyl),
   wherein A₃ is optionally substituted with one or more (such as 1, 2, 3, or more) substituents independently selected from halogen (such as Br, F, I, or Cl), hydroxyl, cyano, nitro, amino, alkyl (such as C₁-C₆ alkyl), alkoxy (such as C₁-C₄alkoxy or methoxy), cycloalkyl (such as C₃-C₈ cycloalkyl), haloalkyl (such as C₁-C₂haloalkyl), haloalkoxy (such as C₁-C₂ haloalkoxy), aryl (such as a C₆ or C₈ aryl), heteroaryl (such as a 5-8 membered heteroaryl), heterocycloalkyl (such as a 3-8 or 5-8 membered heterocycloalkyl), an aryloxy (such as C₆ or C₈ aryloxy or phenoxy), a thioaryl (such as C₆ or C₈ thioaryl or thiophenyl), —CHO, —COOH, —CONR₁₁R₁₂, —COR₁₁, —NR₁₁—CO—R₁₂, —NR₁₁ₐCO—R₁₂ₐ (such as —NH—CO—R₁₂ₐ), and hydrocarbyl having a hydrocarbon chain of 1 to 6 carbon atoms in which the carbon atoms are joined by single, double or triple bonds, and any one carbon atom or any two nonadjacent carbon atoms can be replaced by O, NH, N(C₁-C₄alkyl), or S, and wherein the hydrocarbyl group is optionally substituted with one or more substituents independently chosen from hydroxyl, oxo, halogen, and amino;
R₁₁ₐ is a hydrogen or a C₁₋₃ alkyl;
R₁₂ₐ is a monocyclic heteroaryl (such as a 5-8, 5, 6, or 7 membered heteroaryl), a bicyclic heteroaryl (such as a 9-11 or 10 membered bicyclic heteroaryl), a C₆ or C₈ monocyclic aryl, or a bicyclic aryl (such as a C₉₋₁₁ or C₁₀ bicyclic aryl), wherein the R₁₄ₐ is optionally substituted with one or more (such as 1, 2, or 3) substituents independently selected from halogen (such as Cl, Br, I, or F), C₁₋₃ alkyl (such as methyl), and C₁₋₃ alkoxy (such as methoxy);
R₇, R₈, R₉, R₁₀, R₁₁, and R₁₂ are each independently hydrogen, halogen, hydroxyl, cyano, nitro, amino, —CHO, —CONH₂, —NR₁₃—CO—R₁₄, —CO-aryl (such as benzoyl), —CO-heteroaryl (such as 5-8, 5 or 6 membered heteroaryl), —CO-alkyl (such as acetyl), alkyl (such as C₁-C₆ alkyl), alkoxy (such as C₁-C₄alkoxy), cycloalkyl (such as C₃-C₅ cycloalkyl), haloalkyl (such as C₁-C₂ haloalkyl), haloalkoxy (such as C₁-C₂haloalkoxy), aryl (such as C₆ or C₈ aryl, phenyl, naphthyl, or tolyl), heteroaryl (such as 5-11, 5-8, 5, or 6 membered heteroaryl, furyl, or benzofuryl), heterocycloalkyl (such as 5-11, 5-8, 5, or 6 membered heterocycloalkyl), an aryloxy (such as C₆ or C₈ aryloxy or phenoxy), alkylaryl (such as —CH₂—CH₂-phenyl), or a thioaryl (such as C₆ or C₈ thioaryl or thiophenyl);
R₁₃ and R₁₄ are each independently hydrogen, halogen, hydroxyl, cyano, nitro, amino, —CHO, —CONH₂, —CO-aryl (such as —CO—C₆ or C₈ aryl or benzoyl), —CO-heteroaryl (such as 5-8, 5 or 6 membered heteroaryl), —CO-alkyl (such as —CO—C₁₋₃ alkyl or acetyl), alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), haloalkoxy (such as C₁-C₂haloalkoxy), aryl (such as C₅₋₈, C₅, or C₆ aryl, phenyl, naphthyl, or tolyl), heteroaryl (such as 5-8, 5, or 6 membered heteroaryl, furyl, or benzofuryl), heterocycloalkyl (such as 5-8, 5, or 6 membered heterocycloalkyl), an aryloxy (such as C₆ or C₈ aryloxy or phenoxy), alkylaryl (such as C₁₋₃-C₆ or C₈ aryl or —CH₂—CH₂-phenyl), or a thioaryl (such as C₆ or C₈ thioaryl or thiophenyl), wherein R₁₄ is optionally substituted with one or more (such as 1, 2, or 3) substituents independently selected from halogen (such as Cl, Br, I, or F), C₁₋₃ alkyl (such as methyl), and C₁₋₃ alkoxy (such as methoxy,
wherein two adjacent R groups, R₇ and R₈, R₈ and R₉, or R₉ and R₁₀ are optionally combined together with the carbon atoms they are attached to form a 5-6-membered aromatic ring or 5-8 membered heteroaromatic ring; and
wherein the method is effective in treating, preventing, or ameliorating the disease or disorder associated with a picornavirus infection, or at least one symptom of the disease or disorder associated with a picornavirus infection.

In any aspect or embodiment described herein, (i) A₃ is a C₆ aryl or 6-membered heteroaryl; (ii) A₃ is optionally substituted with 1 or 2 substituents independently selected from halogen (such as Br, F, I, or Cl), —NR₁₁ₐ—CO—R₁₂ₐ (such as —NH—CO—R₁₂ₐ), hydroxyl, cyano, nitro, amino (such as —NH₂), C₁₋₃ alkyl (such as methyl), C₁₋₄ alkoxy (such as methoxy), C₃₋₈ cycloalkyl, C₁₋₂haloalkyl, C₁₋₂haloalkoxy, C₆ aryl, C₈ aryl, 5-8 membered heteroaryl, 3-8 membered heterocycloalkyl (such as 5-8 membered heterocycloalkyl), —CHO, and —COOH; (iii) R₁₁ₐ is a hydrogen or a C₁₋₃ alkyl; (iv) R₁₂ₐ is a 5-8 membered heteroaryl (such as 5, 6, or 7 membered heteroaryl), 9-11 membered bicyclic heteroaryl (such as 10 membered bicyclic heteroaryl), C₆ monocyclic aryl, C₈ monocyclic aryl, or C₉₋₁₁ bicyclic aryl (such as C₁₀ bicyclic aryl), wherein the R₁₄ₐ is optionally substituted with 1, 2, or 3 substituents independently selected from halogen (such as Cl, Br, I, or F), or C₁₋₃ alkyl (such as methyl), and C₁₋₃ alkoxy (such as methoxy); (v) R₇, R₉, and R₁₀ are each hydrogen; (vi) A is N; (vii) B is NR₁₁; (viii) R₁₁ is hydrogen or C₁₋₆ alkyl (such as C₁₋₃ alkyl or methyl); (ix) R₁₃ is a hydrogen or a C₁₋₃ alkyl; (x) R₁₄ is hydrogen, alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), aryl (such as phenyl, naphthyl, or tolyl), heteroaryl (such as furyl, thienyl, or pyrrolyl), and heterocycloalkyl (such as 5-8, 5, or 6 membered heterocycloalkyl); a combination thereof; or (xi) a combination thereof.

In any aspect or embodiment described herein, A₃ is a C₆ aryl or 6-membered heteroaryl having the chemical structure

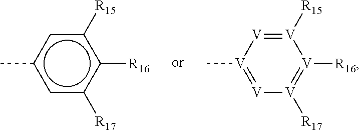

wherein: (i) [symbol] is the point of attachment with Formula II; each V is independently a CH or N, wherein there 0, 1, 2, 3, or 4 (such as 0, 1, 2, or 3) N groups; and (ii) R₁₅, R₁₆, and R₁₇ are independently selected from halogen (such as Br, F, I, or Cl), —NR₁₃ₐCOR₁₄ₐ (such as —NHCOR₁₄ₐ), hydroxyl, cyano, nitro, amino (such as —NH₂), C₁₋₃ alkyl (such as methyl), C₁₋₄alkoxy (such as methoxy), C₃₋₈ cycloalkyl, C₁₋₂haloalkyl, C₁₋₂haloalkoxy, C₆ aryl, C₈ aryl, 5-8 membered heteroaryl, 3-8 membered heterocycloalkyl (such as 5-8 membered heterocycloalkyl), —CHO, and —COOH, wherein (a) one of R₁₅, R₁₆, and R₁₇ is a hydrogen; and (b) one of R₁₅, R₁₆, and R₁₇ is a hydrogen or a C₁₋₆ alkyl (such as C₁₋₃ alkyl or methyl).

In any aspect or embodiment described herein, (i) A₃ is an aryl (such as C₆ or C₈ aryl), a heteroaryl (such as 5-8 membered heteroaryl), a cycloalkyl (such as a C₃₋₈ cycloalkyl group), or a heterocycloalkyl (such as a 5-8 membered heterocycloalkyl); (ii) A₃ is optionally substituted with one or more (such as 1 or 2) substituents independently selected from halogen (such as Br, F, I, or Cl), —NR₁₁ₐ—CO—R₁₂ₐ (such as —NH—CO—R₁₂ₐ), hydroxyl, cyano, nitro, amino (such as —NH₂), C₁₋₃ alkyl (such as methyl), C₁₋₄ alkoxy (such as methoxy), C₃₋₈ cycloalkyl, C₁₋₂ haloalkyl, C₁₋₂haloalkoxy, C₆ or C₈ aryl, 5-8 membered heteroaryl, 3-8 membered heterocycloalkyl (such as 5-8 membered heterocycloalkyl), —CHO, —COOH, and hydrocarbyl having a hydrocarbon chain of 1 to 3 carbon atoms in which the carbon atoms are joined by single, double or triple bonds, and any one carbon atom or any two nonadjacent carbon atoms can be replaced by O, NH, N(C₁-C₄ alkyl), or S, and wherein the hydrocarbyl group is optionally substituted with one or more substituents independently chosen from hydroxyl, oxo, halogen, and amino; (iii) B is CR₁₁R₁₂ or NR₁₁; (iv) R₁₁ and R₁₂ are each independently hydrogen, halogen, hydroxyl, cyano, nitro, amino, —CHO, —CONH₂, C₁₋₃ alkyl (such as methyl), C₁₋₃ alkoxy, C₁₋₂ haloalkyl, and C₁₋₂ haloalkoxy; (v) R₇, R₈, R₉, and R₁₀ are each independently hydrogen, halogen, hydroxyl, cyano, nitro, amino, —CHO, —CONH₂, —NR₁₃—CO—R₁₄, —CO-aryl (such as —CO—C₆ or C₈ aryl or benzoyl), —CO-alkyl (such as —CO—C₁₋₆ alkyl or acetyl), alkyl (such as C₁₋₆ alkyl or methyl), alkoxy (such as C₁₋₄ alkoxy), cycloalkyl (such as C₃₋₈ cycloalkyl), haloalkyl (such as C₁₋₂ haloalkyl), haloalkoxy (such as C₁₋₂ haloalkoxy), aryl (such as a C₆ or C₈ aryl), heteroaryl (such as 5-8, 5, or 6 membered heteroaryl, furyl, thienyl, or pyrrolyl), heterocycloalkyl (such as a 3-8 membered heterocycloalkyl), an aryloxy (such as phenoxy), alkylaryl (such as —CH₂—CH₂-phenyl), or a thioaryl (such as thiophenyl); (vi) R₁₃ and R₁₄ are each independently hydrogen, alkyl (such as C₁-C₆ alkyl), alkoxy (such as C₁-C₄ alkoxy), cycloalkyl (such as C₃-C₅ cycloalkyl), haloalkyl (such as C₁-C₂haloalkyl), aryl (such as phenyl, naphthyl, or tolyl), heteroaryl (such as furyl, thienyl, or pyrrolyl), and heterocycloalkyl (such as 5-8, 5, or 6 membered heterocycloalkyl); or (vii) a combination thereof.

In any aspect or embodiment described herein, either (i) or (ii):
(i) A₃ is an aryl (such as a monocyclic aryl or bicyclic aryl), a heteroaryl (such as a monocyclic heteroaryl or bicyclic heteroaryl), a cycloalkyl (such as a C₃₋₈ cycloalkyl group), or a heterocycloalkyl (such as a 5-8 membered heterocycloalkyl), wherein:
   (a) one carbon atom (such as R₁₅, R₁₆, or R₁₇) is —NR₁₁ₐ—CO—R₁₂ₐ (such as —NH—CO—R₁₂ₐ), cycloalkyl (such as C₃-C₈ cycloalkyl), aryl (such as a C₆ or C₈ aryl), heteroaryl (such as a 5-8 membered heteroaryl), heterocycloalkyl (such as a 3-8 or 5-8 membered heterocycloalkyl), —CHO, or —COOH; and
   (b) a second carbon atom (such as R₁₅, R₁₆, or R₁₇) is optionally substituted with a halogen (such as Br, F, I, or Cl), hydroxyl, cyano, nitro, amino (such as —NH₂), alkyl (such as C₁₋₆ or C₁₋₃ alkyl or methyl), alkoxy (such as C₁₋₄ alkoxy or methoxy), haloalkoxy (such as C₁₋₂ haloalkoxy); or
(ii) R₇, R₈, R₉, and R₁₀ are each independently selected from hydrogen, halogen, hydroxyl, cyano, nitro, amino, —CHO, —CONH₂, —CO-alkyl (such as —CO—C₁₋₆ alkyl or acetyl), alkyl (such as C₁₋₆ alkyl or methyl), alkoxy (such as C₁₋₄ alkoxy), cycloalkyl (such as C₃₋₈ cycloalkyl), haloalkyl (such as C₁₋₂haloalkyl), and haloalkoxy (such as C₁₋₂haloalkoxy),
   wherein R₈ or R₉ (such as R₈) is —NR₁₃—CO—R₁₄, —CO-aryl (such as —CO—C₆ or C₈ aryl or benzoyl), —CO-heteroaryl (such as 5-8, 5 or 6 membered heteroaryl), aryl (such as a C₆ or C₈ aryl), heteroaryl (such as 5-8, 5, or 6 membered heteroaryl, furyl, thienyl, or pyrrolyl), heterocycloalkyl (such as a 3-8 membered heterocycloalkyl), an aryloxy (such as phenoxy), alkylaryl (such as —CH₂—CH₂-phenyl), and a thioaryl (such as thiophenyl).

In any aspect or embodiment described herein, the compound is selected from compounds 51-67.

In an aspect, disclosed is a method of treating or preventing a disease, a disorder, or at least one symptom associated with a picornavirus infection in a subject, the method comprising: providing and administering a therapeutically effective amount of a compound selected from 1-[bis(4-fluorophenyl)methoxy]-3-(5,6-difluoro-1H-1,3-benzodiazol-1-yl)propan-2-ol (compound 108) (4,6-diamino-1,3,5-triazin-2-yl)methyl 3-[5-fluoro-2-(4-fluorophenyl)-1H-indol-3-yl]propanoate (compound 111), 4-(2-{1-[(2-chlorophenyl)methyl]-1H-indol-3-yl}ethenyl)-6-(trifluoromethyl)-1,2-dihydropyrimidin-2-one (compound 139), and N-(3-chlorophenyl)-2-[8-(3-fluorophenoxy)-3-oxo-2H,3H-[1,2,4]triazolo[4,3-a]pyrazin-2-yl]acetamide (compound 167) or a pharmaceutically acceptable salt thereof, or a composition comprising the same to the subject, wherein the method is effective in treating or ameliorating the disease, disorder associated with picornaviruses, or at least one symptom of the disease or disorder associated with picornaviruses.

In any aspect or embodiment described herein, the compound is selected from 1-[bis(4-fluorophenyl)methoxy]-3-(5,6-difluoro-1H-1,3-benzodiazol-1-yl)propan-2-ol (compound 108), (4,6-diamino-1,3,5-triazin-2-yl)methyl 3-[5-fluoro-2-(4-fluorophenyl)-1H-indol-3-yl]propanoate (compound 111), 4-(2-{1-[(2-chlorophenyl)methyl]-1H-indol-3-yl}ethenyl)-6-(trifluoromethyl)-1,2-dihydropyrimidin-2-one (compound 139), and N-(3-chlorophenyl)-2-[8-(3-fluorophenoxy)-3-oxo-2H,3H-[1,2,4]triazolo[4,3-a]pyrazin-2-yl]acetamide (compound 167), or a pharmaceutically acceptable salt thereof, or a composition comprising the same to the subject, wherein the compound, pharmaceutically acceptable salt thereof, or a composition comprising the same, is effective in treating or ameliorating the disease, disorder associated with picornaviruses, or at least one symptom of the disease or disorder associated with picornaviruses.

In an aspect, disclosed is a composition for treating or preventing a disease, a disorder, or symptom associated with picornavirus in a subject comprising a therapeutically effective amount of a compound of Formula I or a pharmaceutically acceptable salt thereof, and/or Formula II or a pharmaceutically acceptable salt thereof, 1-[bis(4-fluorophenyl)methoxy]-3-(5,6-difluoro-1H-1,3-benzodiazol-1-yl)propan-2-ol (compound 108), (4,6-diamino-1,3,5-triazin-2-yl)methyl 3-[5-fluoro-2-(4-fluorophenyl)-1H-indol-3-yl]propanoate (compound 111), 4-(2-{1-[(2-chlorophenyl)methyl]-1H-indol-3-yl}ethenyl)-6-(trifluoromethyl)-1,2-dihydropyrimidin-2-one (compound 139), and/or N-(3-chlorophenyl)-2-[8-(3-fluorophenoxy)-3-oxo-2H,3H-[1,2,4]triazolo[4,3-a]pyrazin-2-yl]acetamide (compound 167) or a pharmaceutically acceptable salt thereof as disclosed herein; wherein the composition is effective in treating or ameliorating the disease, disorder associated with picornaviruses, or at least one symptom of the disease or disorder associated with picornaviruses.

In an aspect, the disease, disorder, symptom, or condition is associated with picornaviruses. In certain embodiments, the disease, disorder, symptom, or condition is associated with enteroviruses. In certain embodiments, the disease, disorder, symptom, or condition is associated with meningitis, encephalitis, acute flaccid myelitis, enterovirus A71 (EV-A71), or a combination thereof. In certain embodiments, the subject is human.

In any aspect or embodiment described herein, the compound of Formula I is selected from Table 1 or a pharmaceutically acceptable salt thereof.

TABLE 1
Exemplary Compounds of Formula I

| Compound | |
|---|---|
| Number | IUPAC Name |
| 1 | N-[4-(1H-benzimidazol-2- |
| | yl)phenyl]acetamide |
| 2 | N-[4-(1H-benzimidazol-2- |
| | yl)phenyl]cyclopropanecarboxamide |
| 3 | 4-(acetylamino)-N-[4-(1H- |
| | benzimidazol-2- |
| | yl)phenyl]benzamide |
| 4 | 4-({[4-(1H-benzimidazol-2- |
| | yl)phenyl]amino}carbonyl)phenyl |
| | acetate |
| 5 | 4-{[4-(5-methyl-1H-benzimidazol- |
| | 2-yl)phenyl]amino}-4-oxobutanoic |
| | acid |
| 6 | N-[4-(1H-benzimidazol-2- |
| | yl)phenyl]benzamide |
| 7 | N-[4-(1H-benzimidazol-2- |
| | yl)phenyl]-2-phenylacetamide |
| 8 | N-[4-(1H-benzimidazol-2- |
| | yl)phenyl]-2-pyridinecarboxamide |
| 9 | N-[4-(1H-benzimidazol-2- |
| | yl)phenyl]-2-(2-thienyl)acetamide |
| 10 | N-[4-(1H-benzimidazol-2- |
| | yl)phenyl]-2-thiophenecarboxamide |
| 11 | N-[4-(1H-benzimidazol-2- |
| | yl)phenyl]-5-bromo-2-furamide |
| 12 | N-[4-(1H-benzimidazol-2- |
| | yl)phenyl]-3-methylbenzamide |
| 13 | N-[4-(1H-benzimidazol-2- |
| | yl)phenyl]-2-fluorobenzamide |
| 14 | N-[4-(1H-benzimidazol-2- |
| | yl)phenyl]-4-fluorobenzamide |
| 15 | N-[4-(1H-benzimidazol-2- |
| | yl)phenyl]-3-fluorobenzamide |
| 16 | N-[4-(1H-benzimidazol-2- |
| | yl)phenyl]-2,3,4,5,6- |
| | pentafluorobenzamide |
| 17 | N-[4-(1H-benzimidazol-2- |
| | yl)phenyl]cyclopentanecarboxamide |
| 18 | N-[4-(1H-benzimidazol-2- |
| | yl)phenyl]-4-(4-morpholinyl)-3- |
| | nitrobenzamide |
| 19 | N-[4-(1H-benzimidazol-2- |
| | yl)phenyl]-2-(4-morpholinyl)-5- |
| | nitrobenzamide |
| 20 | N-[4-(1H-benzimidazol-2- |
| | yl)phenyl]-3-(2-furyl)acrylamide |
| 21 | N-[4-(1H-benzimidazol-2- |
| | yl)phenyl]-2-[(4- |
| | nitrobenzyl)thio]acetamide |
| 22 | N-[4-(1H-benzimidazol-2- |
| | yl)phenyl]-2-(3-4- |
| | phenylthio)acetamide |
| 23 | N-[4-(1H-benzimidazol-2- |
| | yl)phenyl]-5-(3-nitrophenyl)-2- |
| | furamide |
| 24 | N-[4-(1H-benzimidazol-2- |
| | yl)phenyl]-2-(1-naphthyl)acetamide |
| 25 | N-[4-(1H-benzimidazol-2- |
| | yl)phenyl]-2-[(4- |
| | chlorophenyl)thio]acetamide |
| 26 | N-[4-(1H-benzimidazol-2- |
| | yl)phenyl]-5-(4-chlorophenyl)-2- |
| | furamide |
| 27 | N-[4-(1H-benzimidazol-2- |
| | yl)phenyl]-5-(3-chlorophenyl)-2- |
| | furamide |
| 28 | N,N′-bis[4-(1H-benzimidazol-2- |
| | yl)phenyl]terephthalamide |
| 29 | N-[4-(5-methyl-1H-benzimidazol-2- |
| | yl)phenyl]acetamide |
| 30 | N-[4-(6-chloro-1H-benzimidazol-2- |
| | yl)phenyl]acetamide |
| 31 | N-[4-(5-methyl-1H-benzimidazol-2- |
| | yl)phenyl]cyclopropanecarboxamide |
| 32 | N-[4-(6-chloro-1H-benzimidazol-2- |
| | yl)phenyl]propanamide |
| 33 | 2-methyl-N-[4-(5-methyl-1H- |
| | benzimidazol-2- |
| | yl)phenyl]propanamide |
| 34 | 2,2,2-trifluoro-N-[4-(5-methyl-1H- |
| | benzimidazol-2- |
| | yl)phenyl]acetamide |
| 35 | N-[4-(5-methyl-1H-benzimidazol-2- |
| | yl)phenyl]-2-(2-thienyl)acetamide |
| 36 | N-[4-(5-methyl-1H-benzimidazol-2- |
| | yl)phenyl]-2-thiophenecarboxamide |
| 37 | 3,4-dimethoxy-N-[4-(5-methyl-1H- |
| | benzimidazol-2- |
| | yl)phenyl]benzamide |
| 38 | N-[4-(5-methyl-1H-benzimidazol-2- |
| | yl)phenyl]-2-phenoxyacetamide |
| 39 | N-[4-(5-methyl-1H-benzimidazol-2- |
| | yl)phenyl]-2-phenylacetamide |
| 40 | 5-bromo-N-[4-(5-methyl-1H- |
| | benzimidazol-2-yl)phenyl]-2- |
| | furamide |
| 41 | N-[4-(5-methyl-1H-benzimidazol-2- |
| | yl)phenyl]-2-(4- |
| | methylphenoxy)acetamide |
| 42 | N-[4-(5-methyl-1H-benzimidazol-2- |
| | yl)phenyl]-2-(3-7- |
| | phenylthio)acetamide |
| 43 | 3,4-dimethyl-N-[4-(5-methyl-1H- |
| | benzimidazol-2- |
| | yl)phenyl]benzamide |
| 44 | 3-bromo-N-[4-(5-methyl-1H- |
| | benzimidazol-2- |
| | yl)phenyl]benzamide |
| 45 | N-[4-(5-benzoyl-1H-benzimidazol- |
| | 2-yl)phenyl]-2-furamide |
| 46 | N-[4-({[4-(1,3-benzoxazol-2- |
| | yl)phenyl]amino}carbonyl)phenyl]- |
| | 1-benzofuran-2-carboxamide |

In any aspect or embodiment described herein, the compound of Formula I is selected from Table 2 or a pharmaceutically acceptable salt thereof.

TABLE 2
Exemplary Compounds of Formula I

| Compound | |
|---|---|
| Number | IUPAC Name |
| 47 | N-[4-(1H-naphtho[2,3- |
| | d]imidazol-2- |
| | yl)phenyl]acetamide |
| 48 | 3-bromo-N-[4-(1H- |
| | naphtho[2,3-d]imidazol- |
| | 2-yl)phenyl]benzamide |
| 49 | N-[4-(1H-naphtho[2,3- |
| | d]imidazol-2-yl)phenyl]- |
| | 3-phenylpropanamide |
| 50 | N-[3-(1H-naphtho[2,3- |
| | d]imidazol-2-yl)phenyl]- |
| | 2-furamide |

In certain embodiments, the compound of Formula II is selected from Table 3 or a pharmaceutically acceptable salt thereof.

TABLE 3
Exemplary Compounds of Formula II

| Compound | |
|---|---|
| Number | IUPAC Name |
| 51 | N-[2-(5-bromo-2-furyl)-1H- |
| | benzimidazol-5-yl]-4- |
| | nitrobenzamide |
| 52 | N-[2-(5-bromo-2-furyl)-1H- |
| | benzimidazol-5- |
| | yl]acetamide |
| 53 | N-(2-phenyl-1H- |
| | benzimidazol-5- |
| | yl)benzamide |
| 54 | N-[2-(1-methyl-1H-pyrrol- |
| | 2-yl)-1H-benzimidazol-5- |
| | yl]benzamide |
| 55 | N-[2-(5-methyl-2-furyl)- |
| | 1H-benzimidazol-5-yl]-2- |
| | thiophenecarboxamide |
| 56 | N-[2-(2-thienyl)-1H- |
| | benzimidazol-5-yl]-2- |
| | thiophenecarboxamide |
| 57 | N-[2-(2-furyl)-1H- |
| | benzimidazol-5-yl]-2- |
| | thiophenecarboxamide |
| 58 | N-[2-(1-methyl-1H-pyrrol- |
| | 2-yl)-1H-benzimidazol-5- |
| | yl]-2-thiophenecarboxamide |

In any aspect or embodiment described herein, the compound of Formula II is selected from Table 4 or a pharmaceutically acceptable salt thereof.

TABLE 4
Exemplary Compounds of Formula II

| Compound | |
|---|---|
| Number | IUPAC Name |
| 59 | 4-(5-methyl-1H- |
| | benzimidazol-2-yl)aniline |
| 60 | N-[5-(1H-benzimidazol-2- |
| | yl)-2-methylphenyl]-2- |
| | furamide |
| 61 | 4-(6-chloro-1H- |
| | benzimidazol-2-yl)aniline |
| 62 | N-[3-(1H-benzimidazol-2- |
| | yl)phenyl]-2-furamide |
| 63 | N-[3-(6-methyl-1H- |
| | benzimidazol-2-yl)phenyl]- |
| | 2-furamide |
| 64 | 5-bromo-N-[3-(5-methyl- |
| | 1H-benzimidazol-2- |
| | yl)phenyl]-2-furamide |
| 65 | 2,2′-(1,4-phenylene)bis- |
| | 1H-benzimidazole |
| 66 | N-[5-(1H-benzimidazol-2- |
| | yl)-2-methylphenyl]-1- |
| | benzofuran-2-carboxamide |
| 67 | N-[3-(1H-benzimidazol-2- |
| | yl)phenyl]-1-benzofuran-2- |
| | carboxamide |

In any aspect or embodiment described herein, the compound of Formula I is,

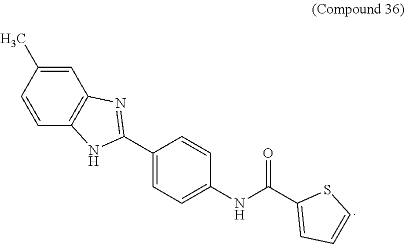 [SMILES: Cc1ccc2[nH]c(-c3ccc(NC(=O)c4cccs4)cc3)nc2c1]

### EXAMPLES

Results

In Silico Screening to Identify Potential 2C Inhibitors

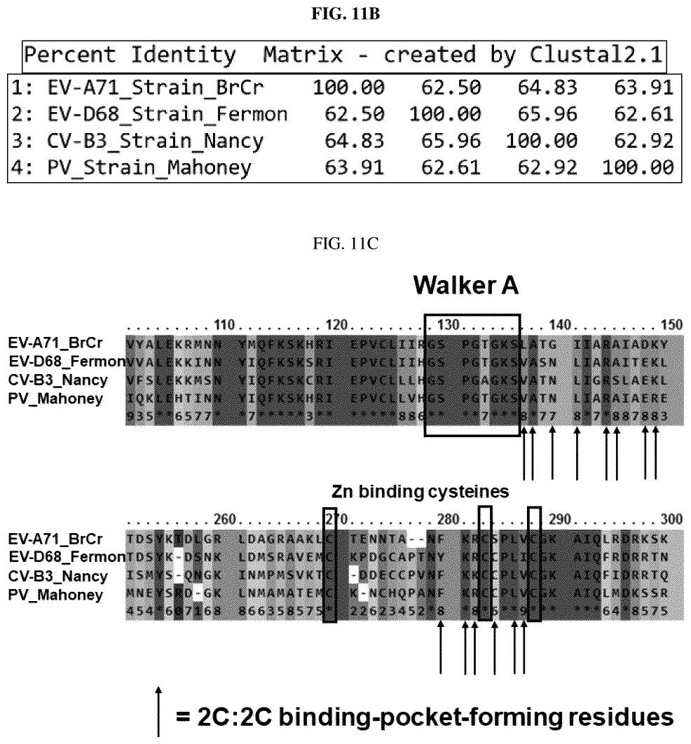

The partial crystal structure of enterovirus A71 (EV-A71) 2C was used for the structure-based drug discovery approach [Guan, H.; et al., Crystal Structure of 2C Helicase from Enterovirus 71. Sci. Adv. 2017, 3 (4), e1602573]. The crystal structure showed the C-terminal α6 helix of one monomer (PDB-5GRB Chain A) protruding into a pocket in the adjacent 2C subunit (PDB—5GRB Chain F) (FIG. 1A). It was suggested that this interaction was necessary for 2C oligomerization which is critical for its multi-functional role in the viral life cycle. Further, on analyzing the 2C protein sequence from four Enteroviruses, it was seen that the residues forming the pocket were conserved (FIG. 11B), making this protein an attractive target for structure-based drug discovery. The terminal 7 amino acid residues (residues 323-329) of the C-terminal α6 helix from Chain A was used as the cognate ligand to select the residues forming the pocket (residues in Chain F). These residues in Chain F were L137, A138, G140, I141, R144, A145, D148, N277, F278, K279, R280, C281, S282, L284, and V285. These residues determined the screening grid box that was generated (FIG. 1B). Using the AtomNet technology, an in silico screening of a curated library of commercially available compounds was carried out. From this screen, 77 compounds were identified and tested in the primary assays for anti-viral activity.

Two Compounds Show In Vitro 2C ATPase Inhibition and EV-A71 Inhibition in Cells

The 77 compounds identified from the in silico screen were purchased from Mcule, Inc. (Palo Algo, California, United States of America) and are shown below in Table 5 and were tested in two primary assays, an ATPase assay to evaluate inhibition of 2C ATPase function and a cell-based assay to study the anti-viral activity of the compounds.

TABLE 5
Exemplary Compounds of the Present Disclosure

| Sr. | |
|---|---|
| No. | IUPAC Name |
| 101 | 4-({[4-(3-chlorophenyl)-5-(pyrrolidin-1-yl)-4H-1,2,4-triazol-3- |
| | yl]sulfanyl}methyl)benzamide, |
| 102 | 2-[4-(1,1,2,3,3,3-hexafluoropropoxy)phenyl]-1,2,3,4-tetrahydroquinazolin-4-one, |
| 103 | 2-amino-4-(isoquinolin-5-yl)-6-(3,4,5-trifluorophenyl)pyridine-3-carbonitrile, |
| 104 | 2-(ethylsulfanyl)ethyl 7-(3,5-difluorophenyl)-5-methyl-4H,7H-[1,2,4]triazolo[1,5- |
| | a]pyrimidine-6-carboxylate, |
| 105 | 5-{[(3-acetyl-2,4,6-trimethylphenyl)methyl]amino}-2,4-difluorobenzamide, |
| 106 | N-[(3-ethyl-1,2-oxazol-5-yl)methyl]-1-(4-fluorophenyl)-4-hydroxy-1H-pyrazole-3- |
| | carboxamide, |
| 107 | 8-ethyl-3-(4-fluorophenyl)-N-(2-hydroxypropyl)thieno[3,2-c]quinoline-2-carboxamide, |
| 108 | 1-[bis(4-fluorophenyl)methoxy]-3-(5,6-difluoro-1H-1,3-benzodiazol-1-yl)propan-2-ol, |
| 109 | {[3-chloro-5-(trifluoromethyl)pyridin-2-yl]carbamoyl}methyl 5-bromopyridine-3- |
| | carboxylate, |
| 110 | 3-[(3-fluorophenyl)methyl]-12-(3,4,5-trifluorophenyl)-3,9- |
| | diazatricyclo[6.4.1.0\hat{ }{4,13}]trideca-1,4(13),5,7,9-pentaen-10-ol, |
| 111 | (4,6-diamino-1,3,5-triazin-2-yl)methyl 3-[5-fluoro-2-(4-fluorophenyl)-1H-indol-3- |
| | yl]propanoate, |
| 112 | 2-amino-4-{4-[2-(1H-imidazol-1-yl)ethoxy]phenyl}-5H,7H,8H-pyrano[4,3-b]pyridine- |
| | 3-carbonitrile, |
| 113 | N-{3-[2-(2-chloro-4-hydroxyphenyl)-1H-imidazol-1-yl]-4-fluorophenyl}acetamide, |
| 114 | N-{[6-(2,5-dimethylphenoxy)pyridin-3-yl]methyl}-2-acetamido-1,3-thiazole-4- |
| | carboxamide, |
| 115 | 3-[(1R)-1-(2,6-dichloro-3-fluorophenyl)ethoxy]-5-[1-(piperidin-4-yl)-1H-pyrazol-4- |
| | yl]pyridin-2-amine, |
| 116 | 2-{5-fluoro-1-[(4-methanesulfinylphenyl)methylidene]-2-methyl-1H-inden-3-yl}-N- |
| | (pyridin-2-yl)acetamide, |
| 117 | 3,4,6-trichloro-N-(4,5-dihydro-1,3-thiazol-2-yl)-1-benzothiophene-2-carboxamide, |
| 118 | 5-(trifluoromethyl)-3-[3-(trifluoromethyl)phenoxy]pyridine-2-carboxamide, |
| 119 | 3-{[(2,5-dichlorophenyl)carbamoyl]methyl}-5-methyl-4-oxo-3H,4H-thieno[2,3- |
| | d]pyrimidine-6-carboxamide, |
| 120 | (4-{[(2-bromo-4-chloro-6-cyanophenyl)amino]methyl}phenyl)urea, |
| 121 | (1R)-1-[6,7-bis(difluoromethoxy)naphthalen-2-yl]-2-methyl-1-(1H-1,2,3-triazol-4- |
| | yl)propan-1-ol, |
| 122 | 5-amino-N-{[2-(2-fluorophenyl)-3-methyl-1H-indol-5-yl]methyl}-1H-pyrazole-3- |
| | carboxamide, |
| 123 | 3-(4-chlorophenyl)-N-[(3,4-difluorophenyl)methyl]-N-methyl-1H-pyrazole-4- |
| | carboxamide, |
| 124 | 3-({[2-hydroxy-3,5-bis(propan-2-yl)phenyl]formamido}methyl)-1,2,4-oxadiazole-5- |
| | carboxamide, |
| 125 | 4-(carbamoylmethoxy)-3-chloro-5-methoxy-N-{3-[3-(trifluoromethyl)phenyl]prop-2-yn- |
| | 1-yl}benzamide, |
| 126 | 2-amino-4-(3-chloro-4-hydroxy-5-methoxyphenyl)-5-methyl-6-(1-methyl-1H-pyrazol-4- |
| | yl)pyridine-3-carbonitrile, |
| 127 | 2-butoxy-N-[2-(diethylamino)ethyl]quinoline-4-carboxamide, |
| 128 | 2-[4-(2-amino-3-cyano-6-cyclobutylpyridin-4-yl)-2-chlorophenoxy]acetamide, |
| 129 | 1-({[2-(6-fluoro-2,4-dihydro-1,3-benzodioxin-8-yl)ethyl]carbamoyl}methyl)-1H-indole- |
| | 3-carboxamide, |
| 130 | 5-(3,5-difluoro-4-methoxyphenyl)-1-[(2S)-1-methoxypropan-2-yl]-4-phenyl-1H- |
| | imidazole, |
| 131 | 3-(2-chloro-6-fluorophenyl)-N-{[3-fluoro-4-(2-methyl-1H-imidazol-1- |
| | yl)phenyl]methyl}-5-methyl-1,2-oxazole-4-carboxamide, |
| 132 | 1-(3,4-difluorophenyl)-1-oxopropan-2-yl 2-methyl-1-phenyl-1H-1,3-benzodiazole-5- |
| | carboxylate, |
| 133 | 3-({methyl[2-methyl-8-(trifluoromethoxy)quinolin-4-yl]amino}methyl)benzamide, |
| 134 | 3-(4-ethoxyphenyl)-8-fluoro-5-[(3-fluorophenyl)methyl]-5H-pyrazolo[4,3-c]quinoline, |
| 135 | 5-{3-[(oxolan-3-yloxy)methyl]phenyl}-4-(trifluoromethyl)pyrimidin-2-amine, |
| 136 | N-(2-{[3-(5-chloro-2-methoxyphenyl)-1,2,4-oxadiazol-5-yl]formamido}ethyl)pyrazine- |
| | 2-carboxamide, |
| 137 | 3-[(5-chloro-1H-1,3-benzodiazol-2-yl)sulfanyl]-4-methyl-1,2,4a,8a-tetrahydroquinolin- |
| | 2-one, |
| 138 | 2-butoxy-N-[2-(diethylamino)ethyl]quinoline-4-carboxamide, |
| 139 | 4-(2-{1-[(2-chlorophenyl)methyl]-1H-indol-3-yl}ethenyl)-6-(trifluoromethyl)-1,2- |
| | dihydropyrimidin-2-one, |
| 140 | 4-(4-bromophenoxy)-N-[5-(difluoromethyl)-1,3,4-thiadiazol-2-yl]-3-fluorobenzamide, |
| 141 | 1-{3′,4′-difluoro-[1,1′-biphenyl]-4-yl}-N-[3-(4-methoxyphenyl)propyl]-1H-imidazole- |
| | 5-carboxamide, |
| 142 | 5-fluoro-2′-methyl-3-(2-methyl-1H-indol-3-yl)-2,3-dihydro-1H,1′H-[3,3′-biindol]-2- |
| | one, |
| 143 | 7-(3,4-dimethoxyphenyl)-1-[4-(trifluoromethyl)phenyl]-1H,4H,5H,6H,7H-imidazo[4,5- |
| | b]pyridin-5-one, |
| 144 | N-benzyl-7H-purin-6-amine, |
| 145 | [(3-carbamoylthiophen-2-yl)carbamoyl]methyl 2-(2,4-dichlorophenyl)acetate, |
| 146 | N-[2-(cyclohex-1-en-1-yl)ethyl]-3-{[6-(3-fluorophenyl)pyrimidin-4-yl]oxy}benzamide, |
| 147 | 4-chloro-5-({[3-fluoro-4-(pyridin-3-yloxy)phenyl]methyl}amino)-2,3-dihydropyridazin- |
| | 3-one, |
| 148 | 5-chloro-6-({[2-(cyclopentyloxy)pyridin-4-yl]methyl}amino)pyridine-3-carboxamide, |
| 149 | 3-(3,4-dichlorophenyl)-3-(5-methoxy-1H-indol-3-yl)propanamide, |
| 150 | N-{6-ethyl-4-oxo-3H,4H-thieno[2,3-d]pyrimidin-3-yl}-2-[4-(pyridin-2- |
| | yloxy)phenoxy]acetamide, |
| 151 | 4-(4-fluorophenyl)-2-{3-[(2-fluorophenyl)methoxy]-2-hydroxypropyl}-1,2- |
| | dihydrophthalazin-1-one, |
| 152 | (1-{[2-(trifluoromethyl)phenyl]methyl}-1,2,3,4-tetrahydroquinolin-5-yl)urea, |
| 153 | N-[4-({2-[(2,4-difluorophenyl)amino]-1,4-dimethyl-1H-1,3-benzodiazol-5- |
| | yl}oxy)pyridin-2-yl]acetamide, |
| 154 | 5-(4-fluorophenyl)-N-{[4-(piperidine-1-carbonyl)phenyl]methyl}thieno[2,3- |
| | d]pyrimidin-4-amine, |
| 155 | N-(2,5-difluorophenyl)-2-[(6-fluoro-2-phenylquinolin-4-yl)oxy]acetamide, |
| 156 | 4-{1-[2-(2-chlorophenyl)ethyl]-1H-imidazol-2-yl}benzamide, |
| 157 | N-(5-cyclopropyl-1,3,4-thiadiazol-2-yl)-5-[(2,3,5,6-tetrafluorophenoxy)methyl]furan-2- |
| | carboxamide, |
| 158 | 2,3-dichloro-N-[2-(2,4-dichlorophenyl)-2-hydroxyethyl]benzamide, |
| 159 | (4-{2-[2-(trifluoromethyl)-1H-1,3-benzodiazol-1-yl]acetyl}phenyl)urea, |
| 160 | 5-(2,6-dichloro-4-nitrophenoxy)pyridine-3-carboxamide, |
| 161 | N-{4-[4-(4-acetylphenyl)piperazine-1-carbonyl]-1,3-oxazol-2-yl}-5-methyl-1,2-oxazole- |
| | 3-carboxamide, |
| 162 | N-(6-{[(3-ethoxypropyl)carbamoyl]amino}-4-methoxy-1,3-benzothiazol-2- |
| | yl)propanamide, |
| 163 | N-[2-(1-ethyl-1H-1,3-benzodiazol-2-yl)ethyl]-7-fluoro-2-methylquinoline-4- |
| | carboxamide, |
| 164 | 3-[(1,2-dimethyl-1H-indol-5-yl)methyl]-1-[4-(trifluoromethyl)phenyl]thiourea, |
| 165 | 7-hydroxy-N-[2-(1H-indol-3-yl)ethyl]-5-oxo-5H-[1,3]thiazolo[3,2-a]pyrimidine-6- |
| | carboxamide, |
| 166 | 3-(2-chloro-6-fluorophenyl)-4-[3-(trifluoromethyl)benzoyl]-1H-pyrrole, |
| 167 | N-(3-chlorophenyl)-2-[8-(3-fluorophenoxy)-3-oxo-2H,3H-[1,2,4]triazolo[4,3-a]pyrazin- |
| | 2-yl]acetamide, |
| 168 | 9-(6-aminopyridin-3-yl)-1-[3-(trifluoromethyl)phenyl]-1H,2H-benzo[h]1,6- |
| | naphthyridin-2-one, |
| 169 | 1-{[4-(2-chloro-4-nitrophenoxy)-3-fluorophenyl]methyl}-3-(3-fluoropropyl)urea, |
| 170 | 2-amino-4-[2-methoxy-5-oxo-6-(propan-2-yl)-5H,6H,7H-pyrrolo[3,4-b]pyridin-3-yl]- |
| | 5H,6H,7H,8H,9H-cyclohepta[b]pyridine-3-carbonitrile, |
| 171 | 4-[2-fluoro-4-({5H,6H,7H,8H-imidazo[1,2-a]pyridin-8-yl}amino)phenoxy]-N- |
| | methylpyridine-2-carboxamide, |
| 172 | 1-ethyl-N-[(8-methoxyquinolin-5-yl)methyl]-N-methyl-1H-indole-5-carboxamide, |
| 173 | N-{[5-(4-fluorophenyl)-1H-pyrazol-4-yl]methyl}-N-methyl-1H-indole-6-carboxamide, |
| 174 | 2,6-difluoro-4-{4-[4-(morpholin-4-yl)phenyl]pyridin-3-yl}phenol, |
| 175 | N-[(3,5-difluorophenyl)methyl]-4-hydroxy-1-(3-methylphenyl)-6-oxo-1H,6H,7H- |
| | pyrazolo[3,4-b]pyridine-5-carboxamide, |
| 176 | [3-(3,4-dimethylphenyl)-1-phenyl-1H-pyrazol-4-yl]methyl 4-chlorobenzoate, |

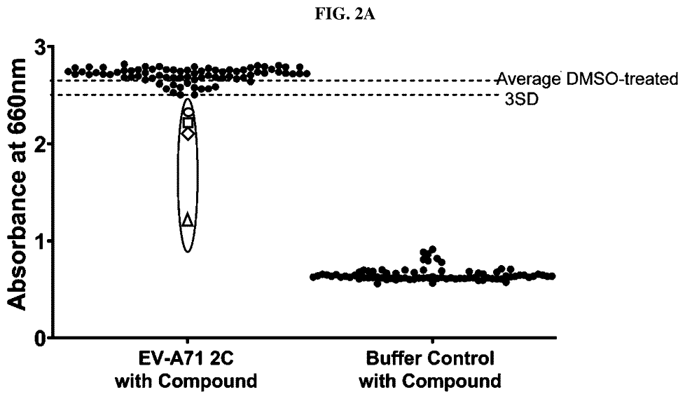

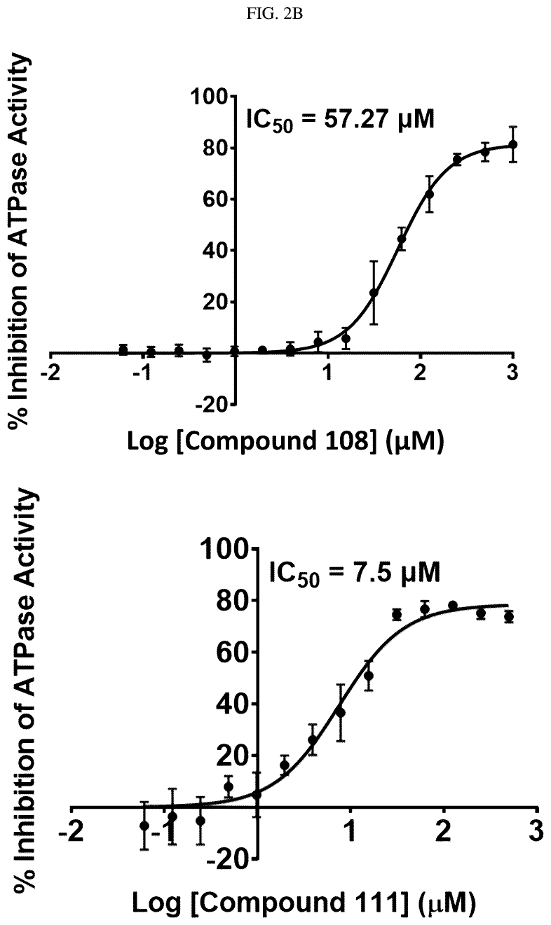

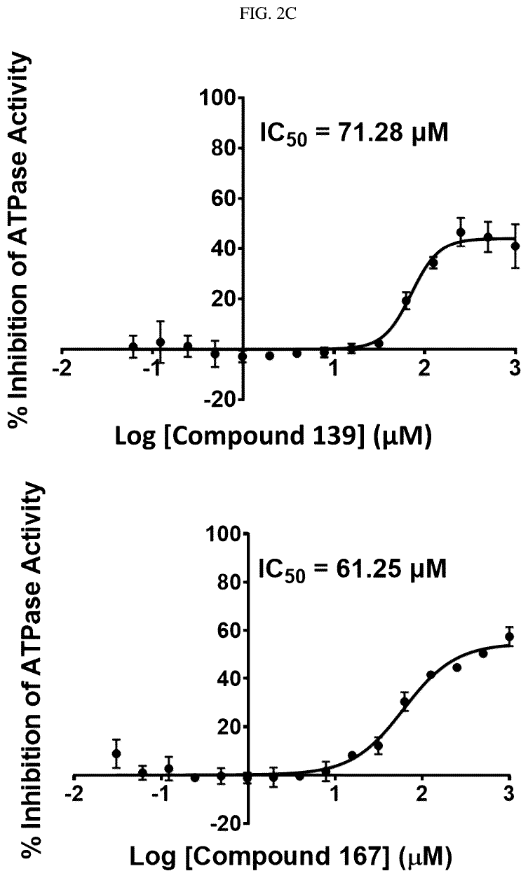

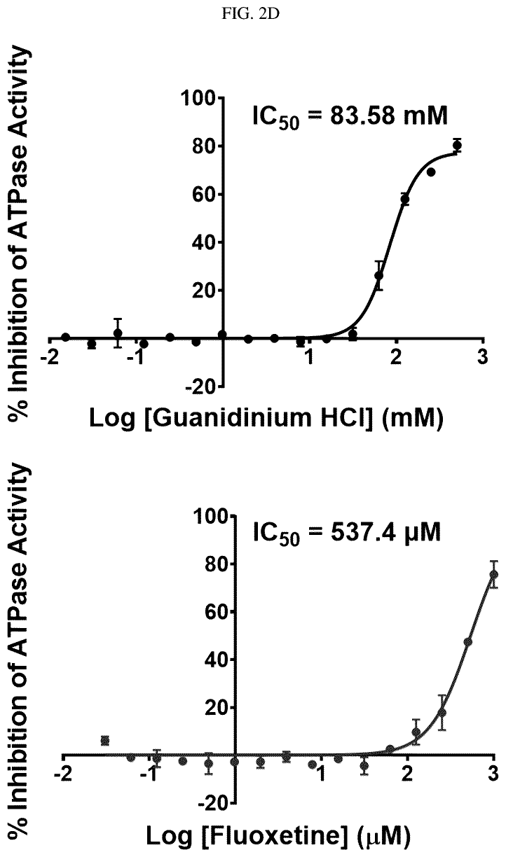

The ATPase assay is a colorimetric assay that uses malachite green to quantify the released inorganic phosphate following adenosine triphosphate (ATP) hydrolysis by 2C³ (FIG. 12). Successful inhibition of 2C reduces production of inorganic phosphate from ATP hydrolysis, resulting in a decrease in absorbance. The 77 compounds were tested in this ATPase assay at a single concentration of 50 μM using the truncated Δ2C¹¹⁶⁻³²⁹. The truncated 2C protein was used for assays since it is far more soluble than the full-length 2C protein. A threshold of 3× standard deviation (3SD) below the average absorbance readout was used to identify 2C ATPase inhibitors. Four compounds, Compound 108, Compound 111, Compound 139, and Compound 167, inhibited the ATPase function of the Δ2C¹¹⁶⁻³²⁹, as indicated by the decrease in absorbance below the 3SD cutoff value of 2.53 for the dimethyl sulfoxide (DMSO)-treated control (FIG. 2A). These four compounds were then studied further by performing a dose response analysis. The IC₅₀ value for this ATPase inhibition was calculated to be in the range of 7 μM to 70 μM (FIGS. 2B-2D and Table 6). Guanidinium hydrochloride (GnHCI) and Fluoxetine have been known to inhibit the ATPase activity of 2C and were used as positive controls in the ATPase assay. The IC₅₀ values for GnHCl was 83.58 mM while that of the racemic mixture of Fluoxetine was 537.4 μM (FIG. 2C). These values are similar to previously published results, validating the assay conditions and the robustness of the assay.

GnHCl has been observed to inhibit the ATPase activity of 2C at IC₅₀ value in the millimolar range. Ulferts et al. observed that even at 500 μM, the racemic mixture of Fluoxetine was unable to inhibit the ATPase activity of 2C by more than 50% [Ulferts, R.; et al., Selective Serotonin Reuptake Inhibitor Fluoxetine Inhibits Replication of Human Enteroviruses B and D by Targeting Viral Protein 2C. Antimicrob. Agents Chemother. 2013, 57 (4), 1952-1956]. The similarity in the IC₅₀ values validates the assay conditions.

TABLE 6
Antiviral activity of the six active compounds (purchased from Hit2Lead.com
(ChemBridge Corporation, San Diego, California, United States of
America)) against EV-A71. The assay was performed three times from which
the 95% confidence interval range for IC₅₀ was calculated using
profile likelihood asymmetrical confidence intervals.

| | | 95% confidence interval | | Selectivity |
|---|---|---|---|---|
| Compound Name* | IC₅₀ (μM) | range (μM) | CC₅₀ (μM) | Index |
| T1 | 5 | 2.661 to 7.907 | 306.2 | 61.24 |
| T2 | 16.98 | 15.08 to 19.58 | 85.36 | 5.03 |
| T3 | 2.9 | 2.274 to 3.434 | >200 | >68.97 |
| T4 | 12.25 | 10.66 to 13.86 | >200 | >16.33 |
| T5 | 102.8 | 76.98 to 152.8 | 61.6 | 0.6 |
| T6 | 44.2 | 41.55 to 47.61 | >200 | >4.52 |

*T1: 2-(2-methoxyphenoxy)-N-[4-(5-methyl-1H-benzimidazol-2-yl)phenyl]acetamide; T2: 4-methoxy-2,5-dimethyl-N-[2-(2-methyl-1H-indol-3-yl)ethyl]benzenesulfonamide; T3: N-[4-(5-methyl-1H-benzimidazol-2-yl)phenyl]-2-furamide; T4: N-[2-(5-bromo-2-furyl)-1H-benzimidazol-5-yl]benzamide; T5: [2-(5-bromo-2-methyl-1H-indol-3-yl)ethyl]aminehydrochloride; T6: N-[2-(3,4-dimethoxyphenyl)ethyl]-2-oxo-2-(2-phenyl-1H-indol-3-yl)acetamide

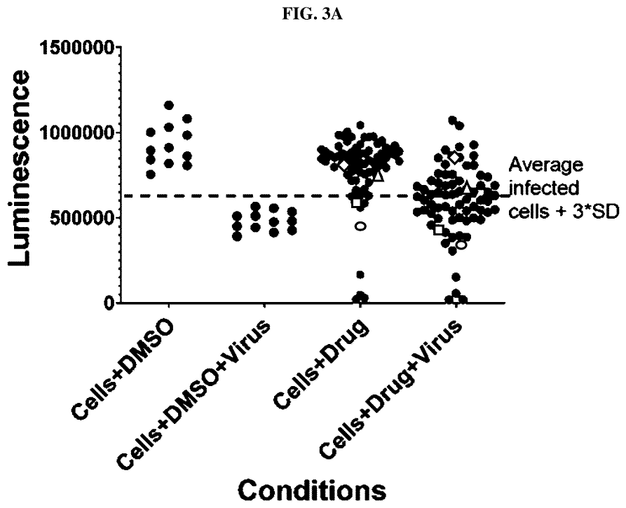

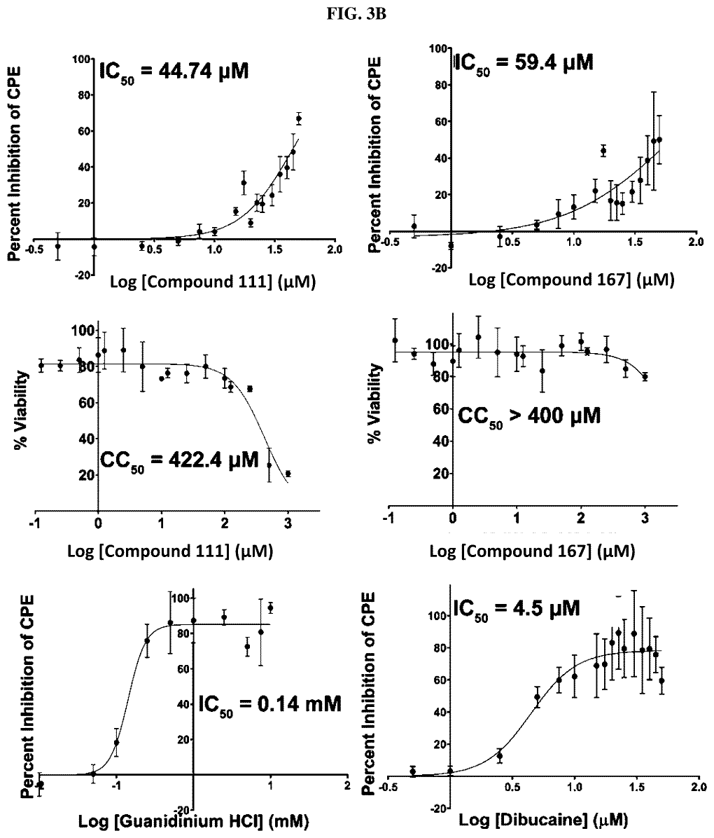

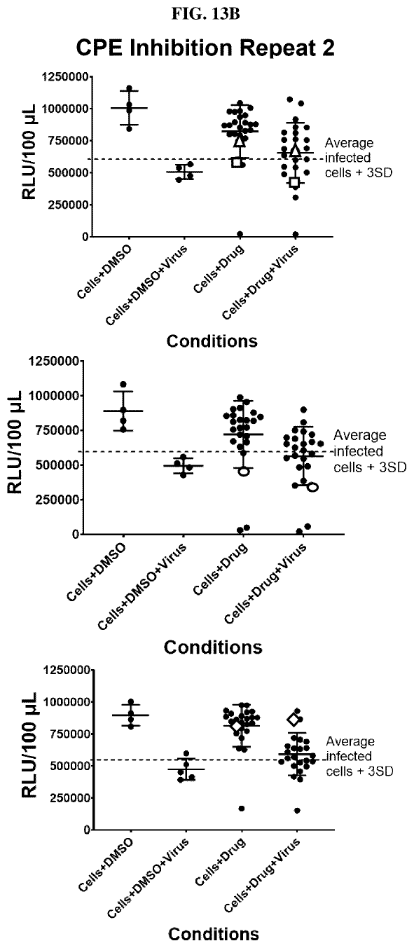

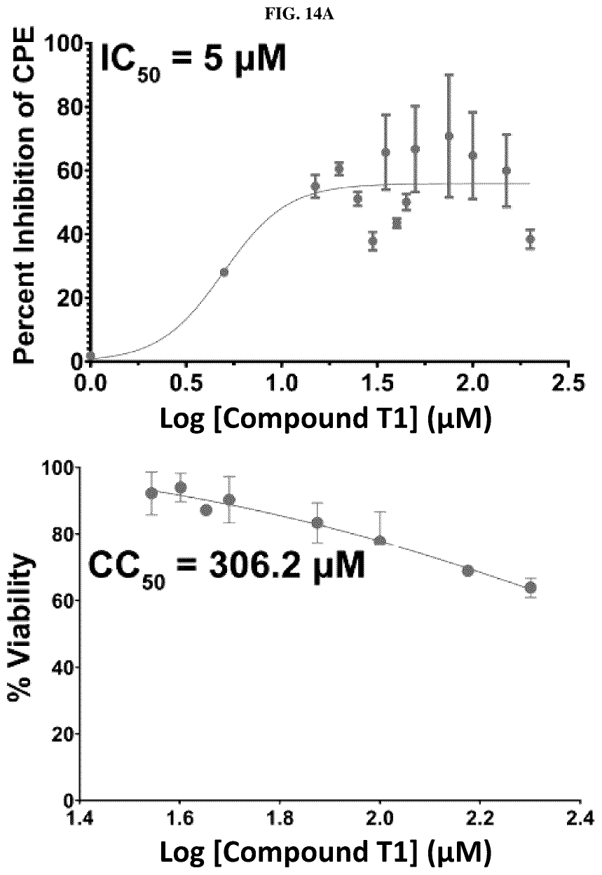

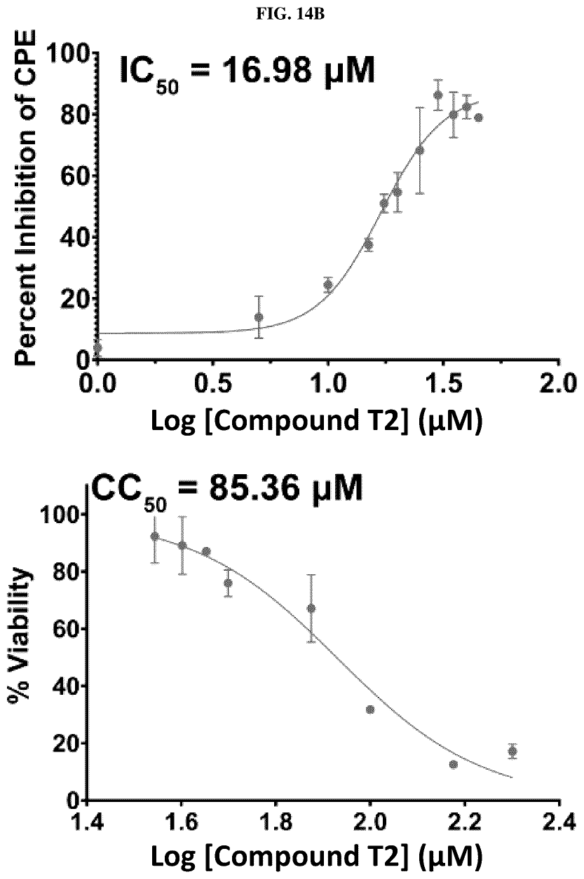

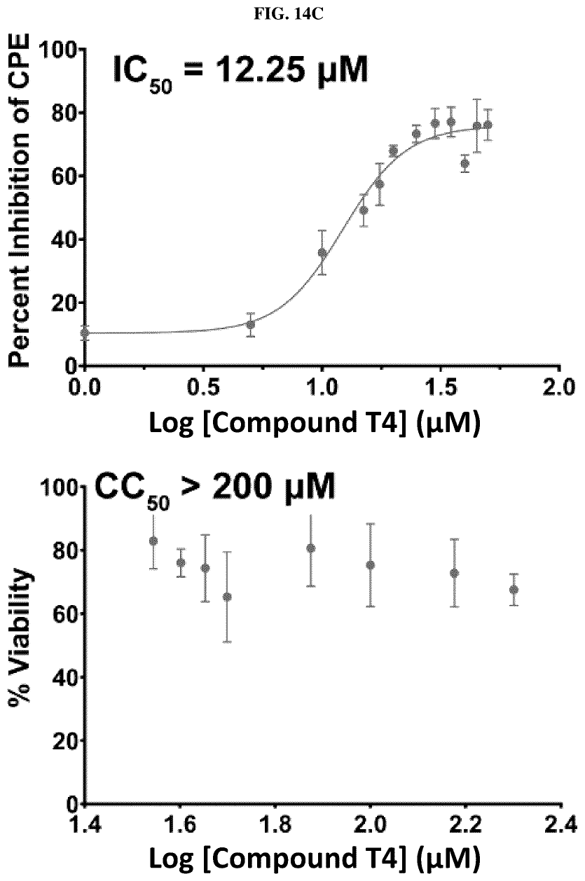

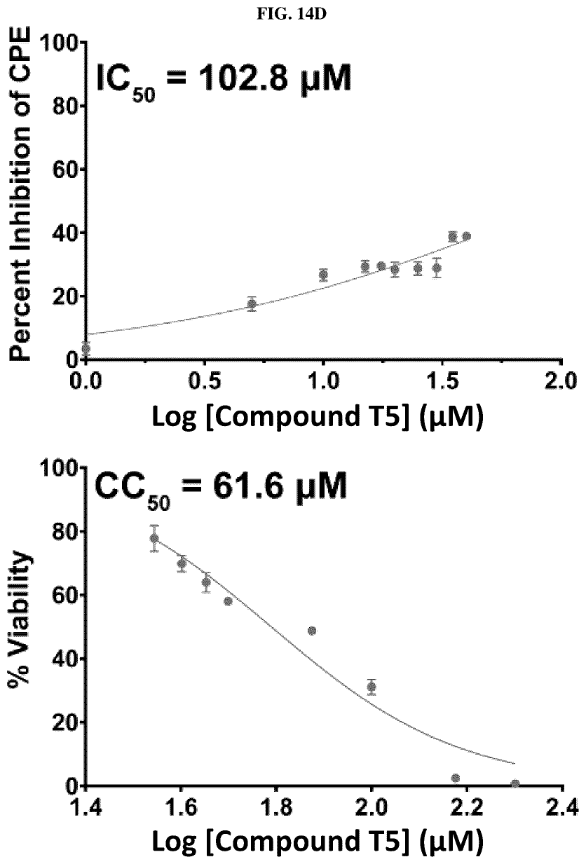

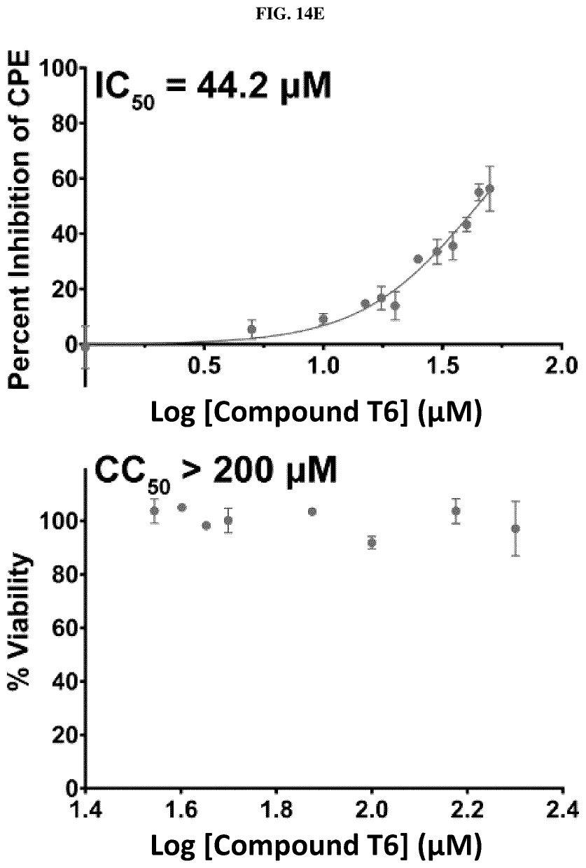

A cell-based assay was performed to assess the anti-viral activity of the 77 compounds against EV-A71 in Vero cells. For this assay, the Cell Titer Glo® One Solution Assay from Promega (Madison, Wisconsin, United States of America) was used to quantify the inhibition of cytopathic effects (CPE) caused by EV-A71. Cells that were infected and lysed by the virus produced lower luminescence values. Conversely, compounds inhibiting the virus would protect the cells from infection and lysis, thereby producing higher luminescence. The 77 compounds were tested at a single concentration of 50 μM for anti-viral activity. A luminescence reading of 3SD above the average luminescence readout of the infected cells (Cells+DMSO+Virus) was used as a threshold to identify anti-viral compounds. The cytotoxicity of the compounds was also tested as seen in the Cells+Drug wells. Two independent repeats were undertaken (FIGS. 13A and 13B). Compounds that resulted in a luminescence reading above the 3SD cutoff in the Cells+Drug wells and the Cells+Drug+Virus wells were selected and using this criteria, 21 of the 77 compounds showed anti-viral activity (FIG. 3A). Out of the four ATPase inhibiting compounds, Compound 111 (unfilled triangle) and Compound 167 (unfilled diamond) inhibited EV-A71 while showing limited toxicity. Compound 111 had a CC₅₀ of 422.4 μM and Compound 167 had a CC₅₀ of >400 μM. Using the IC₅₀ and the CC₅₀ values, the selectivity index for Compound 111 and Compound 167 was determined to be 9.44 and >16.8 respectively (Table 7). The other two compounds identified in the in vitro ATPase assay, Compound 108 (unfilled box) Compound 139 (unfilled circle), were cytotoxic as seen in the Cells+Drug wells that did not show activity above the threshold value in the cell-based assay and so were not used in further experiments (FIG. 3A). Compound 111 and Compound 167 were then evaluated using a dose response study to calculate the IC₅₀ values. Treatment with Compound 111 or Compound 167 inhibited the virus-induced CPE with an IC₅₀ of 44.74 μM or 59.4 μM, respectively (FIG. 3B). For this assay, GnHCl and Dibucaine were used as positive controls since Fluoxetine was observed to be ineffective against EV-A71 at non-toxic concentrations. GnHCl and Dibucaine have been known to be active against the virus in cells with low mM and low μM IC₅₀ values respectively. Here, GnHCl inhibited EV-A71 with an IC₅₀ of 0.14 mM, while Dibucaine treatment resulted in an IC₅₀ of 4.5 μM. These values match known values, validating the experimental conditions and the robustness of the assays (FIG. 3C) [Ulferts, R.; et al., Screening of a Library of FDA-Approved Drugs Identifies Several Enterovirus Replication Inhibitors That Target Viral Protein 2C. Antimicrob. Agents Chemother. 2016, 60 (5), 2627-2638; Shimizu, H.; et al., Mutations in the 2C Region of Poliovirus Responsible for Altered Sensitivity to Benzimidazole Derivatives. J. Virol. 2000, 74 (9), 4146-4154].

TABLE 7
Antiviral activity of active compounds against EV-A71.
The assay was performed three times from which the 95% confidence
interval range for IC₅₀ was calculated using profile likelihood
asymmetrical confidence intervals.

| | | 95% | | |
|---|---|---|---|---|
| | | confidence | | |
| | IC₅₀ | interval | CC₅₀ | Selectivity |
| Compound Name* | (μM) | range (μM) | (μM) | Index |
| 111 | 44.74 | 41.05 to 50.16 | 422.4 | 9.44 |
| 167 | 59.4 | 44.25 to 96.63 | >1000 | >16.8 |

*Compound 111: (4,6-diamino-1,3,5-triazin-2-yl)methyl 3-[5-fluoro-2-(4-fluorophenyl)-1H-indol-3-yl]propanoate; Compound 167: N-(3-chlorophenyl)-2-[8-(3-fluorophenoxy)-3-oxo-2H,3H-[1,2,4]triazolo[4,3-a]pyrazin-2-yl]acetamide

Hit Expansion to Optimize Compound 111 and Compound 167

Having identified two compounds from the initial in silico screen that showed inhibition of 2C ATPase function and inhibition of the virus in a cell-based assay with limited toxicity, a round of hit expansion was performed to identify structure-activity relationships (SAR) for the two identified compounds (Compound 111 and Compound 167) to improve the activity of the compounds (lower IC₅₀ values) as well as their cytotoxicity (higher CC₅₀ values). Analog compounds that were commercially available, so called “analog-by-catalog”, were focused on.

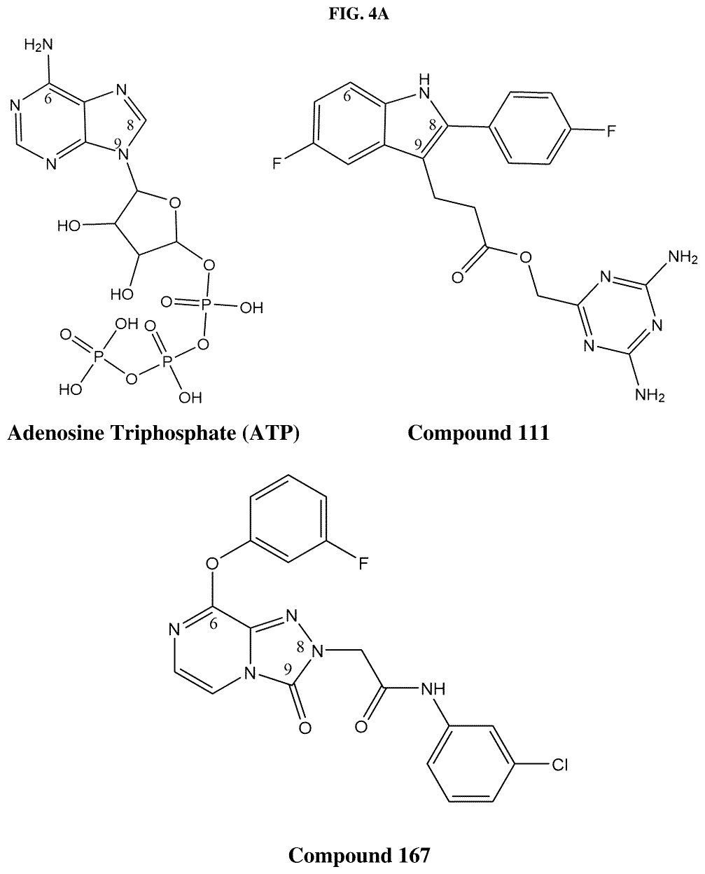

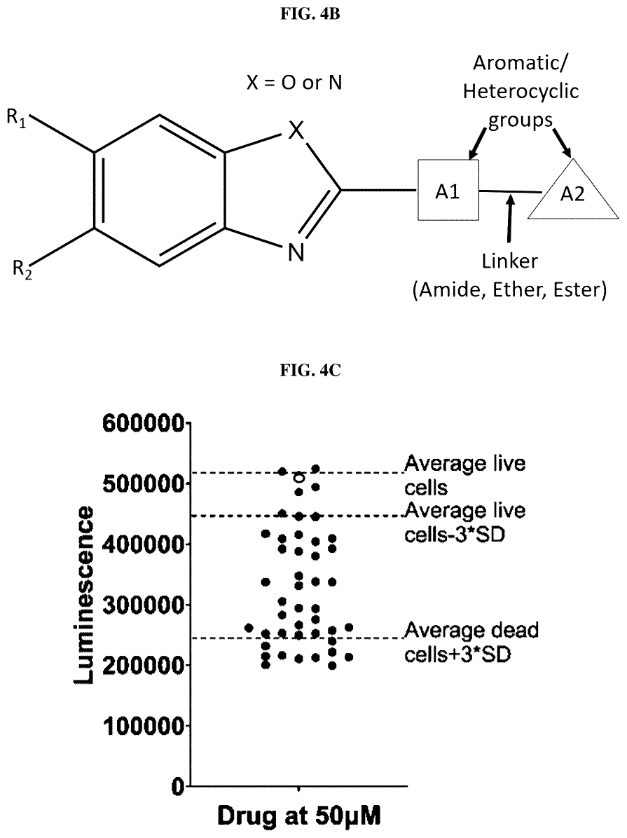

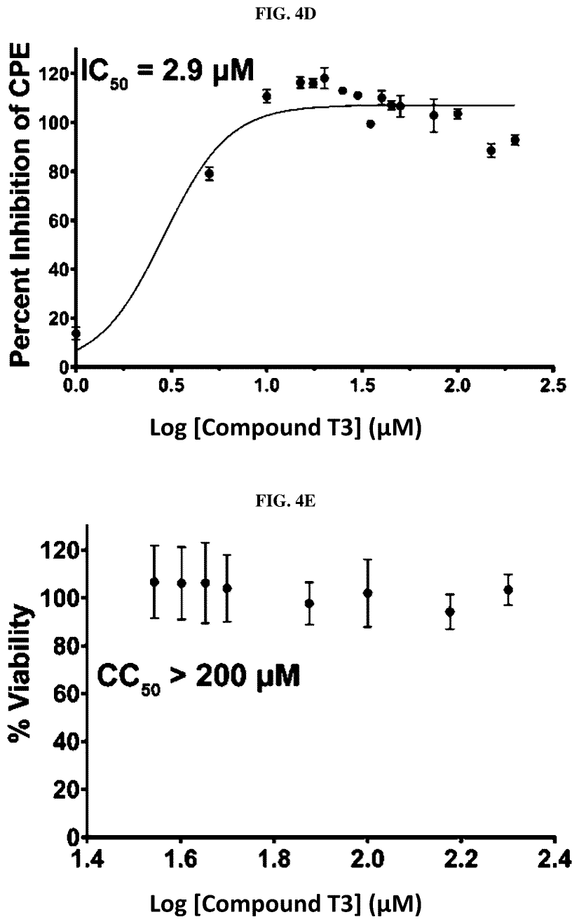

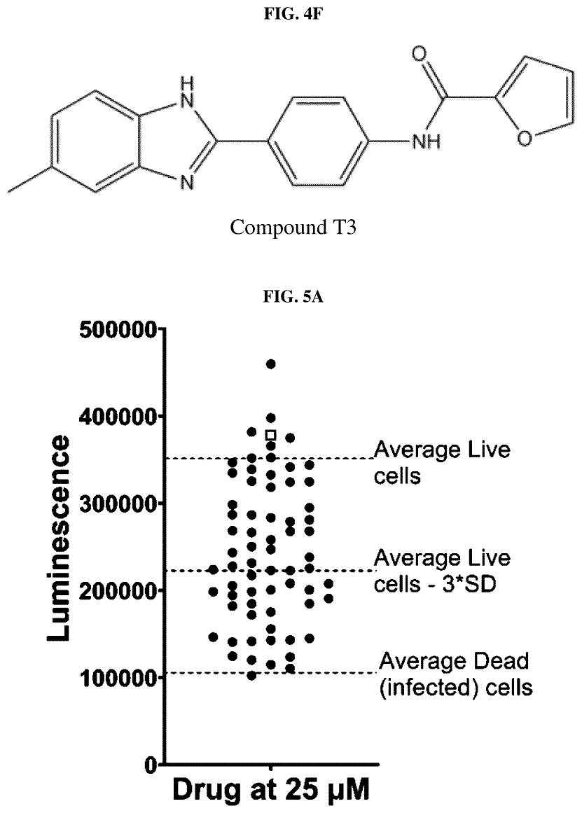

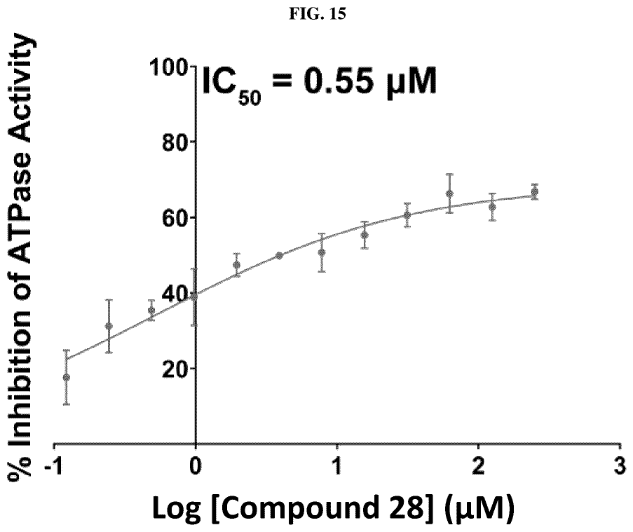

Comparing the structures of Compound 111 and Compound 167 shows a central pharmacophore of a bicyclic ring (FIG. 4A), similar to the benzimidazole of ATP. However, previous studies testing benzimidazole containing compounds against enteroviruses did not show inhibition of the ATPase function of 2C [Shimizu, H.; et al., Mutations in the 2C Region of Poliovirus Responsible for Altered Sensitivity to Benzimidazole Derivatives. J. Virol. 2000, 74 (9), 4146-4154; De Palma, A. M.; et al., The Thiazolobenzimidazole TBZE-029 Inhibits Enterovirus Replication by Targeting a Short Region Immediately Downstream from Motif C in the Nonstructural Protein 2C. J. Virol. 2008, 82 (10), 4720-4730]. To minimize ATP mimicking compounds, analogs with R-groups at positions equivalent to the N⁶ and the N⁹ position of the ATP molecule (numbered respectively) (FIG. 4A) were avoided. Instead, R-groups at the N⁸ position (numbered accordingly) and the 2-position were selected in the analogs to optimize binding to the 2C pocket. To explore this pharmacophore (FIG. 4B), a geometric-based similarity study was performed using binary fingerprints and selected 45 analogs from an in-house library and tested in the cell-based CPE inhibition assay against EV-A71 using Vero cells. Only those compounds that showed luminescence above the 3SD value were considered active. Of the 45 analogs, six compounds (Compounds T1-T6) were active (FIGS. 4C and 15). Upon determining their IC₅₀ and CC₅₀ values using dose response curves, Compound T3 was the best active compound with an IC₅₀ of 2.9 μM (FIG. 4D), and showed no toxicity to Vero cells up to 200 μM resulting in a selectivity index of >68.9 (FIG. 4E). Table 6 shows the IC₅₀, CC₅₀ and selectivity index values of the six active compounds.

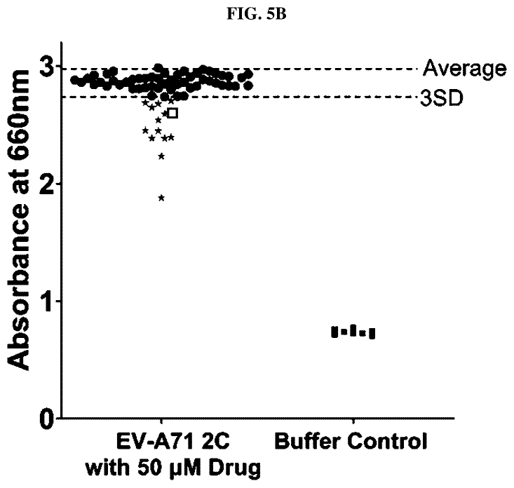

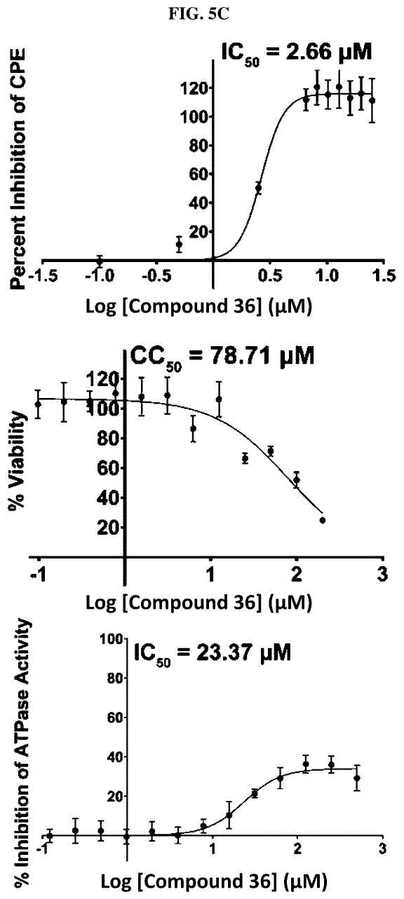

Next, another round of hit expansion was conducted to further identify structure-activity relationships (SAR) for Compound T3 (FIG. 4F). Sixty-seven analogs of Compound T3 were identified to fit the pharmacophore structure and these were grouped into 4 categories based on the pharmacophore model, as shown in FIG. 4B and Tables 4, 6, 7, 8, and 9. The 67 compounds were purchased from Hit2Lead.com (ChemBridge Corporation, San Diego, California, United States of America). The 67 compounds were first tested for anti-viral activity in Vero cells against EV-A71 using the CPE inhibition assay. 41 analogs had a luminescence reading above the 3SD cutoff and were deemed active against the virus (FIG. 5A). Furthermore, dose response curves were generated to determine their respective anti-viral IC₅₀ values and CC₅₀ values to analyze their cytotoxicity. Their antiviral IC₅₀ values ranged from 0.7 μM to 250 μM with 9 analogs having IC₅₀<5 μM (Tables 4, and 6-9). The 67 analogs were also tested for their Δ2C¹¹⁶⁻³²⁹ ATPase inhibition capability using the malachite green based assay. 14 analogs inhibited the ATPase function of Δ2C¹¹⁶⁻³²⁹ (FIG. 5B). Eleven analogs showed anti-viral activity in cells as well as inhibition of the ATPase function of Δ2C¹¹⁶⁻³²⁹ (Table 9). Compound 36 inhibited the virus-induced CPE with IC₅₀ of 2.66 μM (Table 8) and inhibited the ATPase function of Δ2C¹¹⁶⁻³²⁹ with a clear dose-dependent response (Table 7). Further, it had a CC₅₀ value of 78.71 μM giving it a selectivity index of 29.6 (FIG. 5C).

TABLE 8
Analogs inhibiting virus-induced cellular cytopathic effect. The chemical structures of the top nine compounds with IC₅₀ values ≤5 μM
are shown below. The assay was performed three times from which the 95 % confidence interval range for IC₅₀ was calculated
using profile likelihood asymmetrical confidence intervals. Selectivity Index = CC₅₀/IC₅₀.

| | | | 95% confidence | | |
|---|---|---|---|---|---|
| Compound | | IC₅₀ | interval range | CC₅₀ | Selectivity |
| No | Structure | (μM) | (μM) | (μM) | Index |
| 8 | 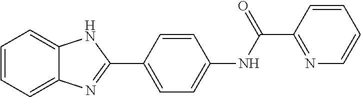 [SMILES: O=C(Nc1ccc(-c2nc3ccccc3[nH]2)cc1)c1ccccn1] | 1.46 | 0.863 to 1.951 | 244.10 | 167.19 |
| 10 | 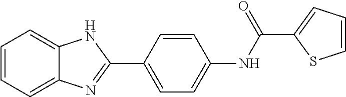 [SMILES: O=C(Nc1ccc(-c2nc3ccccc3[nH]2)cc1)c1cccs1] | 5 | 4.040 to 5.909 | 210.3 | 42.06 |
| 36 | 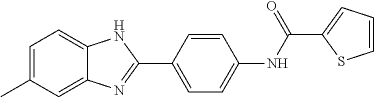 [SMILES: Cc1ccc2[nH]c(-c3ccc(NC(=O)c4cccs4)cc3)nc2c1] | 2.66 | 2.382 to 3.068 | 78.71 | 29.59 |
| 43 | 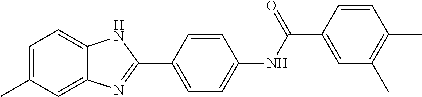 | 4.27 | 3.645 to 5.041 | 170.20 | 39.86 |
| 45 | 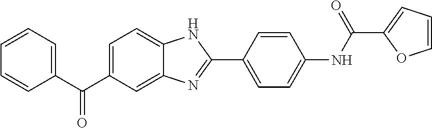 [SMILES: O=C(c1ccccc1)c1ccc2[nH]c(-c3ccc(NC(=O)c4ccco4)cc3)nc2c1] | 4.71 | 3.958 to 5.494 | 59.15 | 12.56 |
| 47 | 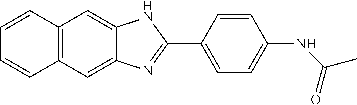 [SMILES: CC(=O)Nc1ccc(-c2nc3cc4ccccc4cc3[nH]2)cc1] | 4.22 | 2.530 to 6.451 | 477.20 | 113.08 |
| 48 | 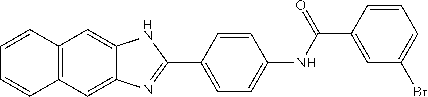 [SMILES: O=C(Nc1ccc(-c2nc3cc4ccccc4cc3[nH]2)cc1)c1cccc(Br)c1] | 0.71 | 0.5501 to 0.8975 | 302.50 | 426.06 |
| 57 | 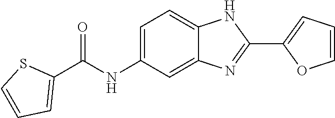 [SMILES: O=C(Nc1ccc2[nH]c(-c3ccco3)nc2c1)c1cccs1] | 2.75 | 2.138 to 3.502 | 8.5 | 3.09 |
| 65 | 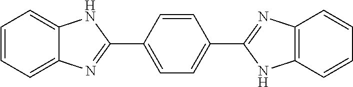 [SMILES: c1ccc2[nH]c(-c3ccc(-c4nc5ccccc5[nH]4)cc3)nc2c1] | 2.17 | 1.390 to 3.206 | 378.7 | 174.52 |

TABLE 9
Analogs inhibiting 2C ATPase activity. IC₅₀ values for ATPase
inhibition were determined for analogs that inhibited EV-
A71 in Vero cells and are as stated below. Compounds 3, 23,
and 41 inhibited 2C ATPase but were not active in cells. The assay
was performed three times from which the 95% confidence interval
range for IC₅₀ was calculated using profile likelihood asymmetrical
confidence intervals.

| | Compound | | 95% confidence |
|---|---|---|---|
| Scaffold # | Number | IC₅₀ (μM) | interval range (μM) |
| Scaffold 1 | 12 | 652.1 | 452.6 to 1076 |
| | 20 | 18.06 | 10.37 to 47.73 |
| | 28 | 0.55 | 0.3392 to 1.239 |
| | 36 | 23.37 | 17.67 to 30.99 |
| | 37 | 20.59 | 15.56 to 29.18 |
| | 40 | 37.26 | 31.21 to 46.08 |
| | 43 | 55.04 | 40.31 to 85.50 |
| | 44 | 18.96 | 15.26 to 23.91 |
| | 46 | 17.35 | 12.46 to 27.57 |
| Scaffold 2 | 48 | 33.07 | 21.97 to 55.43 |
| Scaffold 3 | 51 | 54.09 | 36.76 to 101.8 |

Structure-Activity Relationships for the SJW-2C Compounds

Analysis of the structures of the compounds from the hit expansion showed a number of insights. Compounds in Scaffold 1 (Table 10) suggest that a cyclic alkyl or aromatic moiety at position A₂ would be active against the virus while an aliphatic alkyl chain would require a methyl group at the R₂ position. Also, these compounds suggest that a methylene linker between the amide and A2 aryl group would be detrimental to its anti-viral activity. Further, based on compounds in Table 8, aromatic rings including phenyl, pyridine, thiophene, or furan groups at the A₂ position result in active compounds. Further exploration of this chemical space would be needed to understand the SAR for the side chains at the A₂ and R₂ positions. Additional compounds for Scaffold 2 (Table 11) and Scaffold 4 (Table 13) are needed to establish SAR. Compounds with Scaffold 3 (Table 12) suggest that a methyl group at the 2-position of the furan ring as seen in Compound 55 appears to be unfavorable to the function of that structure since it is absent in Compounds 245, 56, and 57, all of which have activity.

TABLE 10
Antiviral activities of analogs from Scaffold 1*

| Compound | IC₅₀ | 95% confidence | CC₅₀ | Selectivity |
|---|---|---|---|---|
| Number | (μM) | interval range (μM) | (μM) | Index |
| 1 | NA | NA | >400 | NA |
| 2 | 39.34 | 36.07 to 43.38 | 148.40 | 3.77 |
| 3 | NA | NA | >400 | NA |
| 4 | 34.16 | 27.95 to 42.88 | 119.1 | 3.49 |
| 5 | NA | NA | >400 | NA |
| 6 | 30.83 | 25.30 to 39.64 | 13.73 | 0.45 |
| 7 | NA | NA | >400 | NA |
| 8 | 1.46 | 0.863 to 1.951 | 244.10 | 167.19 |
| 9 | 103.80 | 86.16 to 129.6 | 338.30 | 3.26 |
| 10 | 5 | 4.040 to 5.909 | 210.3 | 42.06 |
| 11 | 251.9 | 102.4 to 2259 | 1647 | 6.54 |
| 12 | 13.21 | 12.63 to 13.79 | 432.10 | 32.71 |
| 13 | NA | NA | 98.1 | NA |
| 14 | NA | NA | 133 | NA |
| 15 | NA | NA | 29.22 | NA |
| 16 | NA | NA | >400 | NA |
| 17 | 13.36 | 11.78 to 16.62 | 266.00 | 19.91 |
| 18 | NA | NA | 11.11 | NA |
| 19 | 7.28 | 6.222 to 8.501 | 2093 | 287.50 |
| 20 | 5.41 | 4.451 to 6.481 | >400 | >73.94 |
| 21 | NA | NA | >400 | NA |
| 22 | NA | NA | 760.90 | NA |
| 23 | NA | NA | 1.14 | NA |
| 24 | 8.34 | 7.274 to 10.47 | 58.82 | 7.05 |
| 25 | NA | NA | 582.3 | NA |
| 26 | 5.70 | 4.302 to 10.43 | 44.27 | 7.77 |
| 27 | 5.85 | 5.303 to 6.358 | 67.60 | 11.56 |
| 28 | 41.68 | 34.25 to 54.60 | >400 | >9.6 |
| 29 | 69.28 | 60.09 to 85.66 | >400 | >5.77 |
| 30 | NA | NA | >400 | NA |
| 31 | 9.6 | 8.353 to 10.84 | >400 | >41.67 |
| 32 | NA | NA | >400 | NA |
| 33 | 11.85 | 10.23 to 13.42 | 467.6 | 39.46 |
| 34 | NA | NA | 617.8 | NA |
| 35 | NA | NA | >400 | NA |
| 36 | 2.66 | 2.382 to 3.068 | 78.71 | 29.59 |
| 37 | 69.1 | 57.53 to 87.40 | >400 | >5.79 |
| 38 | NA | NA | >400 | NA |
| 39 | NA | NA | >400 | NA |
| 40 | 81.88 | 63.50 to 115.4 | >400 | >4.89 |
| 41 | NA | NA | >400 | NA |
| 42 | 6.99 | 6.206 to 7.883 | 684.40 | 97.91 |
| 43 | 4.27 | 3.645 to 5.041 | 170.20 | 39.86 |
| 44 | 10.50 | 8.248 to 12.80 | 475.30 | 45.23 |
| 45 | 4.71 | 3.958 to 5.494 | 59.15 | 12.56 |
| 46 | 40.04 | 37.92 to 42.46 | 1381.00 | 34.49 |

*NA: Anti-viral activity below 3 standard deviation cutoff

TABLE 11
Antiviral activities of analogs from Scaffold 2*

| Compound | IC₅₀ | 95% confidence | CC₅₀ | Selectivity |
|---|---|---|---|---|
| Number | (μM) | interval range (μM) | (μM) | Index |
| 47 | 4.22 | 2.530 to 6.451 | 477.20 | 113.08 |
| 48 | 0.71 | 0.5501 to 0.8975 | 302.50 | 426.06 |
| 49 | 22.06 | 19.58 to 24.79 | 154.20 | 6.99 |
| 50 | NA | NA | 562.3 | NA |

*NA: Anti-viral activity below 3 standard deviation cutoff

TABLE 12
Antiviral activities of analogs from Scaffold 3*

| Compound | IC₅₀ | 95% confidence | CC₅₀ | Selectivity |
|---|---|---|---|---|
| Number | (μM) | interval range (μM) | (μM) | Index |
| 51 | 72.39 | 61.37 to 91.76 | 126.30 | 1.74 |
| 52 | 7.1 | 6.332 to 7.968 | 9.2 | 1.30 |
| 53 | 13.23 | 12.11 to 14.38 | 124.80 | 9.43 |
| 54 | 47.64 | 38.09 to 60.85 | 33.1 | 0.69 |
| 55 | NA | NA | >400 | NA |
| 56 | 20.00 | 18.85 to 21.35 | 123.40 | 6.17 |
| 57 | 2.75 | 2.138 to 3.502 | 8.5 | 3.09 |
| 58 | 23.65 | 22.51 to 24.86 | 62.84 | 2.66 |

*NA: Anti-viral activity below 3 standard deviation cutoff

TABLE 13
Antiviral activities of analogs from Scaffold 4*

| Compound | IC₅₀ | 95% confidence | CC₅₀ | Selectivity |
|---|---|---|---|---|
| ID | (μM) | interval range (μM) | (μM) | Index |
| 59 | NA | NA | 189.2 | NA |
| 60 | NA | NA | >400 | NA |
| 61 | 51.33 | 48.07 to 56.15 | 126.1 | 2.46 |
| 62 | 50.25 | 45.71 to 57.63 | 81.52 | 1.62 |
| 63 | 24.75 | 22.44 to 27.25 | 130.70 | 5.28 |
| 64 | 71.44 | 56.51 to 111.2 | 43.75 | 0.61 |
| 65 | 2.17 | 1.390 to 3.206 | 378.7 | 174.52 |
| 66 | 8.52 | 7.909 to 9.114 | 94.69 | 11.11 |
| 67 | 14.91 | 14.04 to 15.81 | 43.02 | 2.89 |

*NA: Anti-viral activity below 3 standard deviation cutoff

Compound 36 Binds to Recombinant EV-A71 2C Protein In Vitro

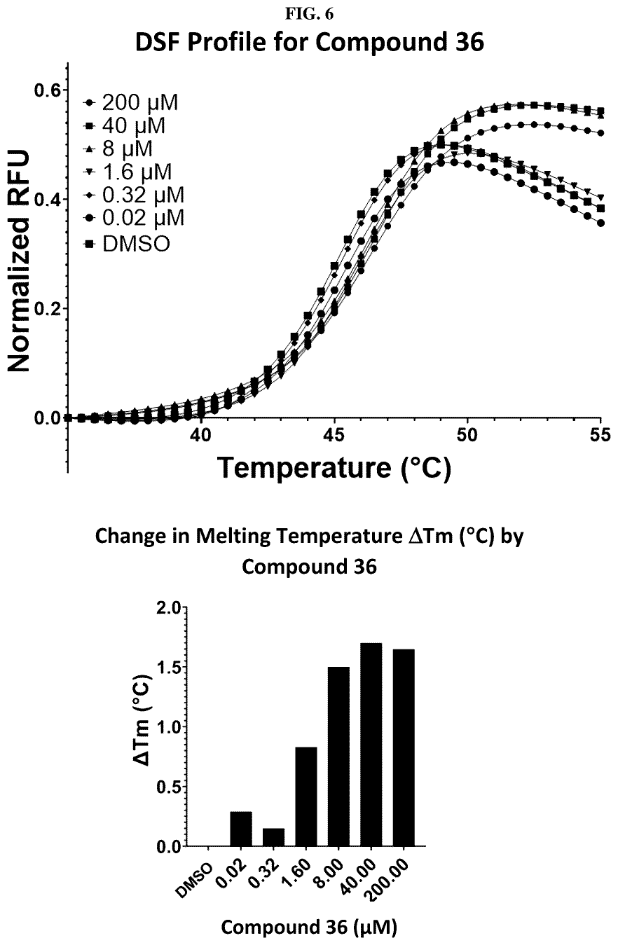

Differential scanning fluorimetry (DSF) was used to study the direct binding of Compound 36 to EV-A71 Δ2C¹¹⁶⁻³²⁹. Using this technique, an increase in the melting temperature I of the target protein is observed if a compound binds to and stabilizes it. DSF has been used to show small molecule interactions with recombinant 2C. A dose-dependent increase in the Tₘ was detected when Compound 36 was incubated with Δ2C¹¹⁶⁻³²⁹ (FIG. 6) at a range from 0.02 μM to 200 μM. The dose-dependent shift in Tₘ indicates that the compound directly interacted with the Δ2C¹¹⁶⁻³²⁹ and that the inhibition of the ATPase function of Δ2C¹¹⁶⁻³²⁹ is likely as a result of this direct interaction.

Escape Mutants are Located in the Capsid Proteins

An escape mutant assay was employed to provide insight into the binding site and mode of action of the compounds. Resistance to two compounds, Compound 167 (parent compound) and Compound 36, was selected by serially passaging EV-A71 in the presence of increasing concentrations of the compounds. After 15 passages, viral genomes were extracted and sequenced. DMSO treated virus served as a negative control and all the genome sequences from the drug treated viruses were compared to the DMSO treated EV-A71.

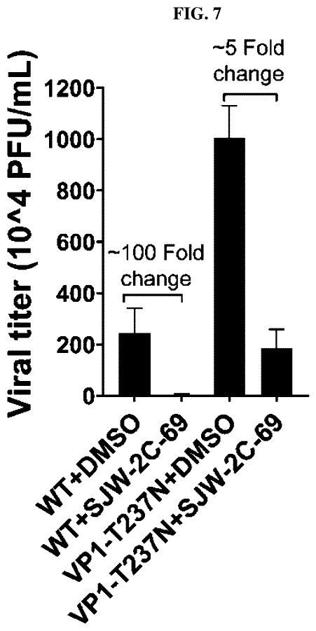

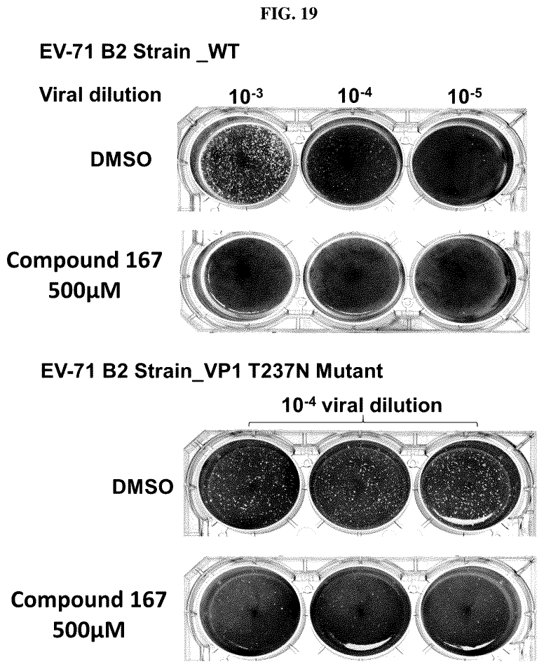

Passaging with the parent compound, Compound 167, selected one mutation, T237N, in the VP1 capsid protein. To validate whether this mutation resulted in resistance, VP1-T237N was reverse engineered into the viral genome, and the mutant EV-A71-TN was generated. Comparative analysis using plaque assays, showed that the mutant has similar infectivity to the wild type EV-A71 (WT), producing plaques of similar size and shape (FIG. 19). In the presence of Compound 167, EV-A71-TN showed ˜5-fold decrease in viral titer, while the wild-type EVA-71 showed ˜100-fold decrease in the viral titer (FIG. 7) indicating that the T237N mutation in VP1 offers partial resistance to Compound 167. Passaging with Compound 36 selected mutations in VP4 (K47E) and 3A (R34W).

Compound 36 does not Inhibit the EV-A71 Replicon System

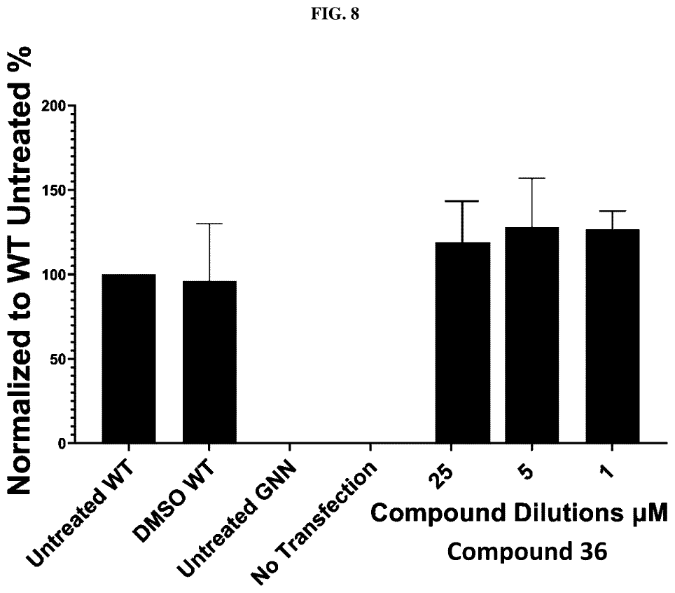

The DSF assay showed direct binding of the compounds to 2C, while the escape mutant assay suggested that the capsid proteins are involved in the mechanism of inhibition. It was therefore hypothesized that the compounds should have little or no inhibition of a replicon system that lacks the structural capsid proteins. To address this, an EV-A71 replicon system was used in which the P1 region, that encodes for the capsid proteins, is replaced with the sequence encoding for a fluorescent reporter protein whilst maintaining cleavage boundaries to ensure correct polyprotein processing. Replicon assays are effective for investigation of replication in real-time, independent of other aspects of the viral lifecycle, such as entry, packaging and egress. In vitro transcribed WT and 3D^{pol}-GNN (a replication defective replicon harboring GDD>GNN mutation of the polymerase active site) EV-A71 replicon RNAs were transfected into HeLa cells and exposed to 1, 5 or 25 μM Compound 36. Addition of the compound at all tested concentrations showed no statistically significant difference in the number of reporter positive cells when compared with the untreated control (FIG. 8).

Molecular Docking of Compound 167 and Compound 36 in the 2C:2C Binding Pocket

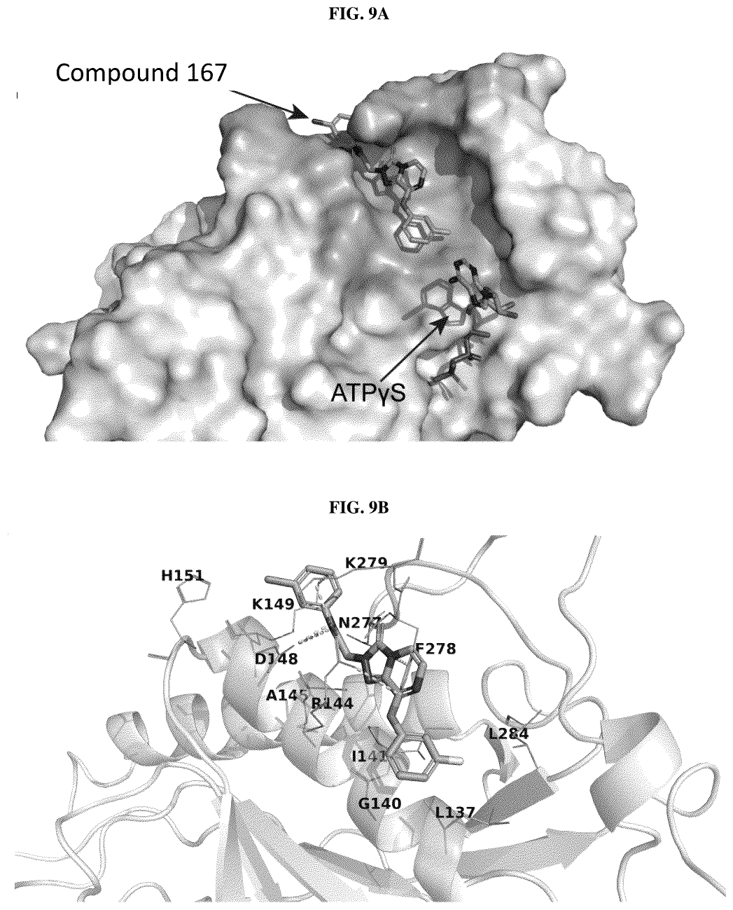

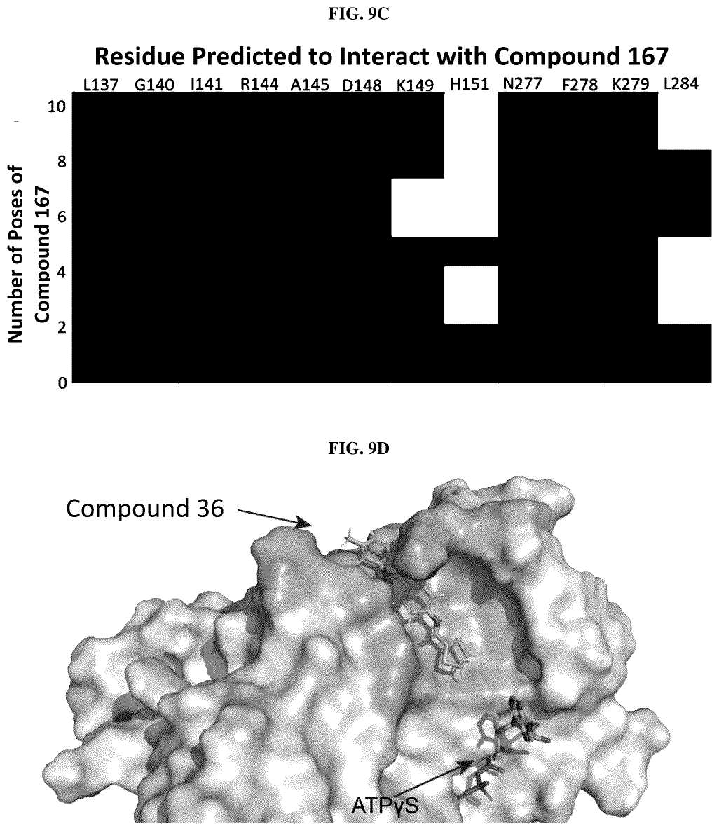

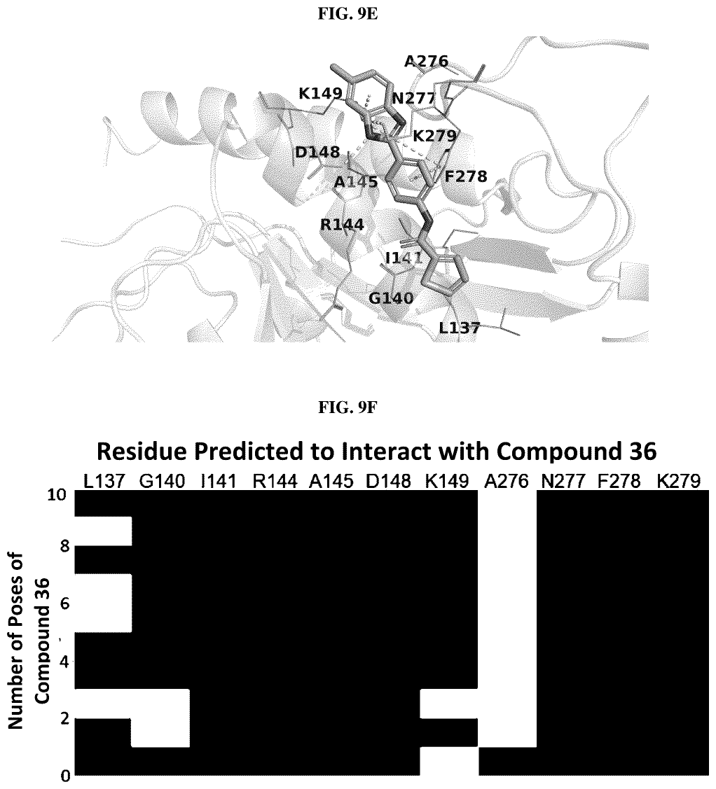

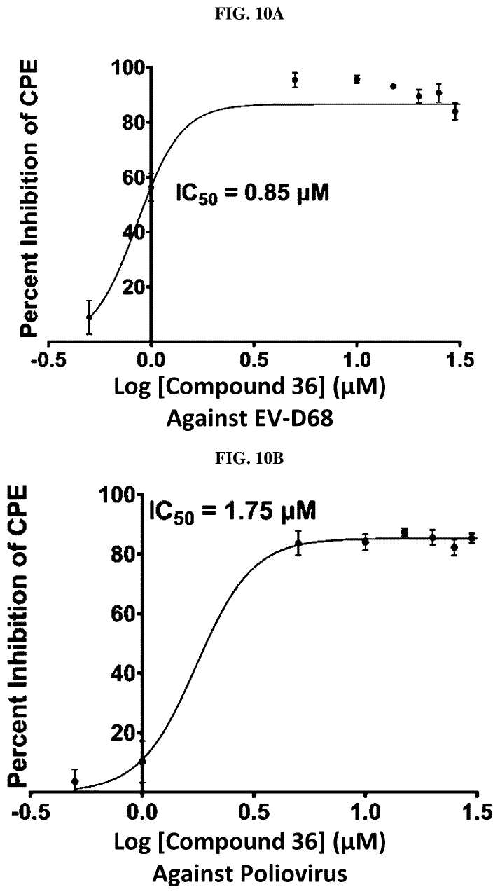

Having carried out an extensive hit expansion and having the DSF and ATPase assays indicate direct interaction between the two compounds and Δ2C¹¹⁶⁻³²⁹, a prediction as to the residues interacting with these compounds was desired. The parent compound (Compound 167) and Compound 36 were docked in the 2C:2C binding pocket using Glide docking to identify potential intermolecular interactions with specific residues in this pocket to understand if the virus would be able to mutate these residues to develop resistance. Using the Induced Fit docking application with expanded sampling, 64 poses for Compound 167 and 51 poses for Compound 36 in the pocket were generated. This program is useful because it accounts for the flexibility of the ligand as well as the defined binding pocket. FIGS. 9A and 9D show the top pose for Compound 167 and Compound 36 respectively and FIGS. 9B and 9E show the residues forming the pocket interactions with the compounds. Using the top 10 poses of each ligand and using the Interaction Fingerprints panel in Maestro, the common residues interacting with the two compounds were identified (FIGS. 9C and 9F).

The top ranked poses for the two compounds have a similar orientation and multiple contacts to residues in the pocket. Further, the binding site of these ligands is near the ATP binding site but does not overlap with it. The common residues interacting with Compound 167 and Compound 36 are L137, I141, R144, A145, D148, K149, A276, N277, F278 and K279. Residues L137, I141, A145 and A276 are predicted to contribute to hydrophobic interactions with the compounds. R144 and K279 are predicted to form pi-cation interactions, while F278 forms pi-pi interaction with the compounds. D148 is predicted to form hydrogen bonds with either the carboxyl group of the amide moiety in Compound 167 or the benzimidazole ring of Compound 36. L137 and I141 are immediately downstream of the Walker A motif. Further, in the absence of compound R144 is suggested to form a salt bridge with E235 of the adjacent 2C subunit and help in the oligomerization of 2C. Wang et al., showed that mutating residues 148, 149 and 150 from poliovirus to alanine (E148A/R149A/E150A) resulted in temperature sensitive virus [Wang, C.; et al., A C-Terminal, Cysteine-Rich Site in Poliovirus 2CATPase Is Required for Morphogenesis. J. Gen. Virol. 2014, 95 (Pt 6), 1255-1265; Wang, C. Functional Analysis of Poliovirus Protein 2CATPase in Viral RNA Replication and Encapsidation Using Alanine Scanning Mutagenesis. 2011]. Although the previous data confirmed that E150A was alone capable of resulting in temperature-sensitivity, no further information was provided for the effects of the other two residues. N277 is conserved across the four representative enterovirus species (FIG. 11). K279 has been shown to be important at the encapsidation stage of the viral life cycle. A majority of the residues interacting with Compound 167 and Compound 36 appear to be important for the virus suggesting that mutating these residues might not produce viable virus.

Compound 36 is Active Against EV-D68 and Poliovirus

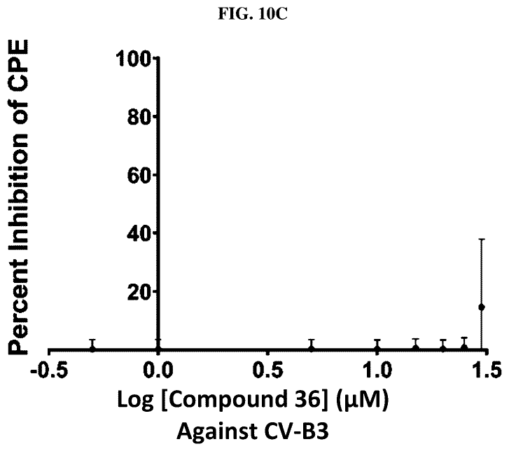

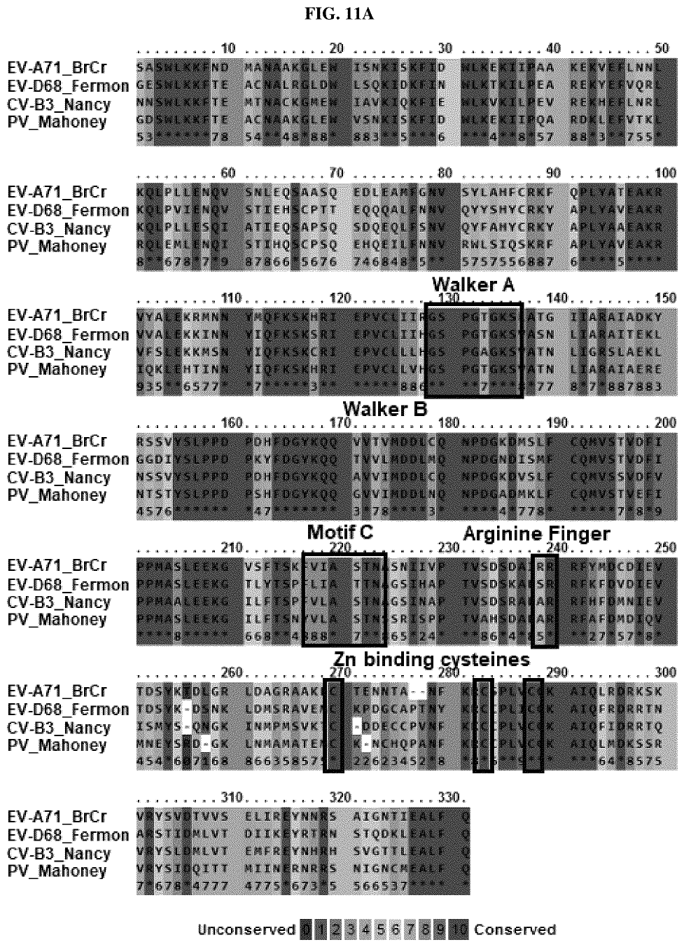

A broad-spectrum anti-viral agent is highly desirable against EVs, especially because of the high number of serotypes. 2C is well conserved among EVs, and thus the compounds should have broad activity. Compound 36 was tested against EV-D68, poliovirus Mahoney strain, and coxsackievirus-B3 (CV-B3). FIG. 10 shows that Compound 36 is highly potent against EV-D68 with an IC₅₀ value of 0.85 μM and against PV Mahoney strain with an IC₅₀ of 1.7 μM. However, it did not inhibit CV-B3.

The Antiviral Program for Pandemics program (NIAID) was also used to screen and validate the anti-viral results for Compound 36 against a wider selection of viruses using a cytopathic effect assay: EV-A71, EV-D68, PV-1, CV-B3, Eastern equine encephalitis virus, human echovirus-11, human echovirus-30, influenza A (H1N1), West Nile virus, and Middle East Respiratory Syndrome. Compound 36 was only active against EVA-71 and EVD-68 in this assay (Table 14) and showed no activity against the other viruses. Compound 36 had an EC₅₀ of 4.2 μM against EV-A71 and 0.39-0.52 μM against EV-D68. A secondary screen using a Virus Yield Reduction Assay was then carried out against EV-A71 and EV-D68. This is a two-step assay where first the virus is produced in the cell containing the compounds and then the viral titer is measured. This assay confirmed the anti-viral activity of Compound 36. It had an EC₅₀ of 1.7 μM against EV-A71 and 0.52 μM against EV-D68 (Table 15).

TABLE 14
Primary assay to test the antiviral activity of Compound
36through the Antiviral Program for Pandemics from NIAID.
EV, Enterovirus; CPE, Cytopathic effects; Tox, Toxicity;
SI₅₀ = CC₅₀/EC₅₀
PRIMARY ASSAY

| Virus | Compound | | EC₅₀ | CC₅₀ | |
|---|---|---|---|---|---|
| Name | Number | Drug Assay Name | μM | UM | SI₅₀ |
| EV-71 | 36 | Visual (CPE/Tox) | >32 | 32 | 0 |
| | 36 | Neutral Red (CPE/Tox) | 4.7 | 42 | 8.9 |
| EV-68 | 36 | Visual (CPE/Tox) | 0.52 | 24 | 46 |
| | 36 | Neutral Red (CPE/Tox) | 0.39 | 9 | 23 |

TABLE 15
Secondary assay to test the antiviral activity of Compound
36through the Antiviral Program for Pandemics from
NIAID. EV, Enterovirus; CPE, Cytopathic effects; Tox, Toxicity;
VYR, Virus Yield Reduction; SI₅₀ = CC₅₀/EC₅₀; SI₉₀ = CC₅₀/EC₉₀
SECONDARY ASSAY

| Virus | Compound | | EC₅₀ | EC₉₀ | CC₅₀ | | |
|---|---|---|---|---|---|---|---|
| Name | Number | Drug Assay Name | μM | μM | μM | SI₅₀ | SI₉₀ |
| EV-71 | 36 | Visual (VYR)/ | | 3.2 | 27 | | 7.8 |
| | | Neutral Red Tox | | | | | |
| | 36 | Neutral Red | 1.7 | | 25 | 15 | |
| | | (CPE/Tox) | | | | | |
| EV-68 | 36 | Visual (VYR)/ | | 0.59 | 9.4 | | 15.9 |
| | | Neutral Red Tox | | | | | |
| | 36 | Neutral Red | 0.52 | | 3.5 | 6.7 | |
| | | (CPE/Tox) | | | | | |

### DISCUSSION

Enteroviruses are a major health burden globally but despite over 70 years of research efforts, no licensed small-molecule anti-EV therapeutics exist. 2C is highly conserved across the EVs and is an essential protein due to its involvement at multiple critical stages in the viral life cycle. These properties make 2C a good target for broad-spectrum anti-viral therapeutics. Over the past four decades, several compounds have been identified that target 2C. To our knowledge, the compounds described in this paper are the first small molecules screened and selected to target the 2C:2C interaction pocket.

Based on the X-ray structure of the EV-A71 Δ2C¹¹⁶⁻³²⁹, the C-terminal α6 helix of one 2C subunit protrudes into the 2C:2C binding pocket of the adjacent subunit and it was suggested that this interaction was important for the oligomerization of 2C. Since 2C needs to oligomerize for ATPase activity, the inhibition of the ATPase activity served as the primary assay to determine whether the compounds were binding to the predicted 2C:2C interaction pocket. Inhibition of 2C ATPase function together with a dose-dependent increase in the Tₘ indicates that the compound binds directly to 2C. 2C is likely to exist in different conformational states during ATP hydrolysis, RNA binding, and helicase activities and it has been observed to exist in open and closed states. Compound 36 may preferentially bind to one state to partially inhibit ATPase function without completely inhibiting the ability of 2C to oligomerize and hydrolyze ATP.

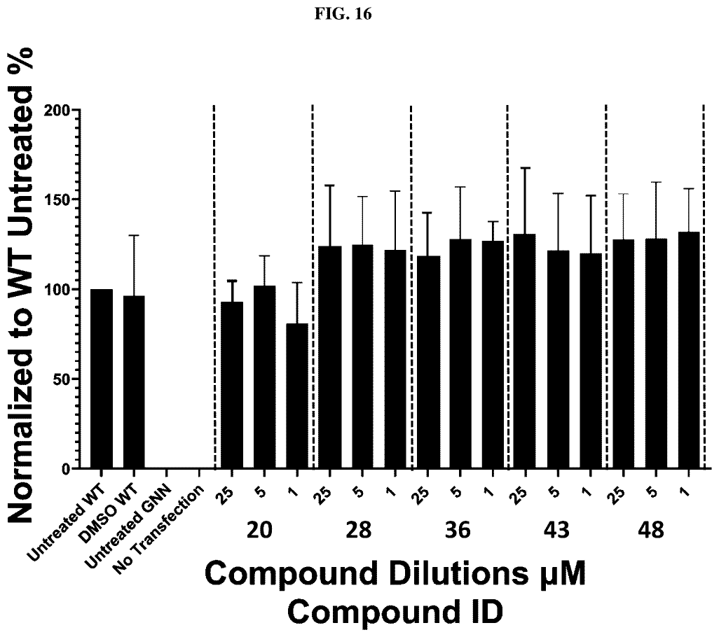

Even the most potent Δ2C¹¹⁶⁻³²⁹ ATPase inhibitor identified in this study, Compound 28 (IC₅₀=0.55 μM), had a maximum ATPase inhibition of ˜60-65% at relatively high concentrations (FIG. 15). This suggests that the inhibition of the ATPase activity of 2C may not be the main mechanism of action for its potent anti-viral activity observed in cells. Since 2C plays an important role in RNA replication, the effect of viral RNA replication by this family of compounds was evaluated using the replicon system. It was observed that the compounds do not affect the EV-71 replicon system (FIG. 16).

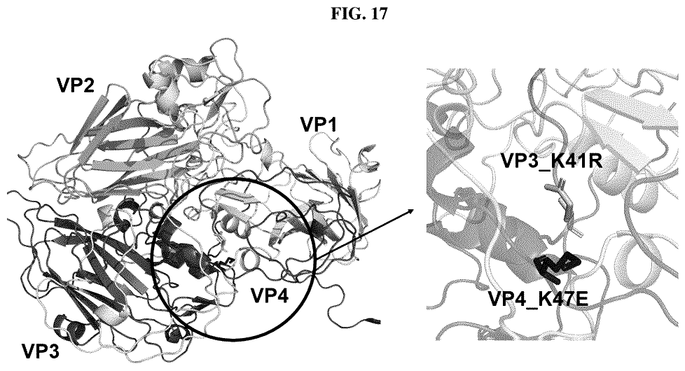

Molecular docking studies show the orientation of the compound in the pocket and identified potential interacting residues. The pocket forming residues are conserved amongst the enteroviruses (FIG. 11) and so to escape the inhibition by compounds, the virus is more likely to select suppressor mutations in other proteins that are either directly affected by the compounds or in proteins that interact with 2C to avoid negative impact on its overall fitness. Consistent with this hypothesis, virus passaged in the presence of Compound 167 and Compound 36 evolved mutations in the capsid proteins VP1 (T237N) and VP4 (K47E), as well as the non-structural 3A protein (R34W). A quasi-infectious virus with an impaired encapsidation phenotype was rescued partially by a single mutation in either 2C (N252S) or in the capsid protein VP3 (E180G) but fully rescued by the double mutant [Liu, Y.; et al., Direct Interaction between Two Viral Proteins, the Nonstructural Protein 2CATPase and the Capsid Protein VP3, Is Required for Enterovirus Morphogenesis. PLoS Pathog. 2010, 6 (8), e1001066]. This interaction between 2C and VP3 was also demonstrated biochemically using co-immunoprecipitation assays. Another study showed that K279A/R280A in PV 2C produced encapsidation defective phenotypes [Wang, C.; et al., Alanine Scanning of Poliovirus 2CATPase Reveals New Genetic Evidence That Capsid Protein/2CATPase Interactions Are Essential for Morphogenesis. J. Virol. 2012, 86 (18), 9964-9975]. Interestingly, K279 in EV-A71 forms a predicted pi-cation bond with Compound 36 (FIG. 9E). One of the rescue mutations from the Wang et al. study for this defect was T36I in the capsid protein VP1 but this mutant virus was temperature sensitive. For different clones, K41R in VP3 along with C323R in 2C or N203S in VP1 with C323R were also able to rescue the encapsidation defect. These studies provide strong evidences that 2C interacts with the capsid proteins and plays a critical role in the morphogenesis of Enteroviruses. Interestingly, residue 41 in EV-A71 VP3 is already an arginine and it is in proximity to the K47E mutation observed here in VP4 (FIG. 17). Although these locations are based on the mature virus conformations, the region harboring these residues might be important for the virus encapsidation steps. A model has been proposed by others where replication organelle-membrane-bound 2C interacts with the capsid pentamer through its interaction with VP3 and the pentamer then interacts with the VPg-linked viral genomic RNA that eventually leads to the assembly of progeny virus [Liu, Y.; et al., PLoS Pathog. 2010, 6 (8), e1001066]. It is also known that RNA loading of the capsids occurs while being tethered to the membrane. Further, it is known that 2C and 3A along with VP1 and 3D, all co-localized to membrane clusters suggestive of replication organelles. The role 3A plays in shuttling lipids between lipid droplets and replication organelles is well known. Yeast two-hybrid and mammalian two-hybrid systems have demonstrated interactions between 3A and 3AB with 2B, 2C and 2BC. Interestingly, one of the escape mutations to Compound 36 was in 3A-R34W.

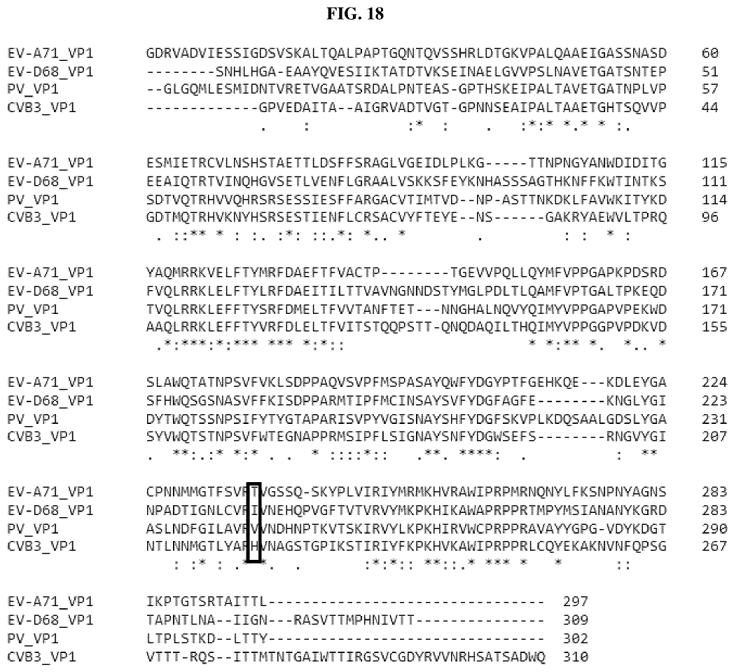

Although the 2C:2C binding pocket is highly conserved, Compound 36 was not active against CV-B3. Further, the escape mutant, VP1-T237N, was able to offer resistance to the parent compound, Compound 167. Upon aligning the VP1 sequences from EV-A71, EV-D68, PV and CV-B3, the equivalent position is either an isoleucine in EV-D68, or a valine in PV or a histidine in CV-B3 (FIG. 18). Histidine at this location has been implicated to be responsible for the nuclear localization of CV-B3. Since this histidine at this position is conserved among coxsackievirus-B1 (CV-B1) through coxsackvirus-B6 (CV-B6), Compound 36 may be effective against other CVs and used as a broad-spectrum antiviral.

Taken together, the results of this study suggest that the family of compounds might be inhibiting the interaction between 2C and the capsid proteins. Further, structural biology efforts are currently being conducted to determine a structure of the compound bound to 2C to aid the medicinal chemistry efforts to develop broad-spectrum antiviral agents with a novel chemistry against viruses that affect millions worldwide.

Methods

Cell Lines, Viruses, Compounds and Chemicals

African Green Monkey kidney cells (Vero), HeLa H1 and RD cells were purchased from American Type Culture Collection (ATCC, Manassas, Virginia, United States of America). All the cell lines were cultured in Dulbecco's modified Eagle's medium (DMEM, Gibco, Waltham, Massachusetts, United States of America) supplemented with 10% fetal bovine serum (FBS, Gibco, Waltham, Massachusetts, United States of America) and 1× Anti-Anti (Gibco, Waltham, Massachusetts, United States of America). All cell lines were maintained at 37° C. in 5% CO₂. Enterovirus A71 (EV-A71), poliovirus (PV) Mahoney Strain, enterovirus D68 (EV-D68), and coxsackievirus-B3 (CV-B3) were purchased from ATCC (Manassas, Virginia, United States of America). EV-A71 and PV were grown and amplified in Vero cells, EV-D68 was grown and amplified in Rhabdomyosarcoma (RD) cells and coxsackvirus-B3 (CVB3) was grown and amplified in HeLa H1 cells. Virus titers were determined by end point titration using plaque assay. Guanidinium hydrochloride, Dibucaine and Fluoxetine were purchased from Sigma (Saint Louis, Missouri, United States of America). For recombinant protein production, the E. coli was grown in 2× yeast extract tryptone medium (YT medium). The 2×YT medium was prepared by mixing 16 g of tryptone (Sigma, Saint Louis, Missouri, United States of America), 10 g of yeast extract (Sigma, Saint Louis, Missouri, United States of America), 5 g of sodium chloride (Fisher Scientific, Waltham, Massachusetts, United States of America) and 2 g of glucose (Fisher Scientific, Waltham, Massachusetts, United States of America) in 1 L of de-ionized water and then sterilized by autoclaving for 30 minutes at 15 psi (1.05 kg/cm²) on liquid cycle.

In Silico Screening

The available 2C structure (PDB: 5GRB) is a 2.8 Å crystal structure in which six 2C molecules fill the asymmetric unit. The oligomerization of 2C has been observed to be important for its ATPase function, but it has been unclear whether the crystal contacts observed in the available structure are truly representative of the solution structure conformation. On comparing Chains A-F of the PDB structure, there were six very different interfaces observed in the asymmetric unit, and only Chain F showed ATP binding in a reasonable orientation. Guan et al. built a model of the 2C hexamer but did not share the coordinates and suggested that it was very approximate. Thus, chain F binding pocket was used as the protein receptor for virtual screening, with residues 323-329 of chain A serving as the prototype cognate binding ligand. The residues of the receptor pocket include: L137, G140, I141, I142, R144, A145, D148, A267, K268, L269, N277, F278, K279, R280, C281, S282, L284, V285. Prior to screening, the ATP ligand, solvent, and ions were removed. A curated molecular library of several million compounds was screened. Top scoring compounds were clustered and subsequently filtered for favorable properties to arrive at a final subset of 77 deliverable compounds.

2C Viral Protein Purification

EV-A71 Δ2C (amino acids 116-329) was designed with an N-terminal maltose-binding protein (MBP) tag and a C-terminal hexa-His tag. The DNA fragment encoding the protein was codon optimized for Escherichia coli (E. coli) protein expression and synthesized synthetically by GenScript (Piscataway, New Jersey, United States of America) before being cloned into the pMALc5× vector. The protein was produced in the E. coli Rosetta™ 2(DE3) pLysS cells (Millipore Sigma, Saint Louis, Missouri, United States of America). The cells were grown in the 2×YT media containing 100 μg/mL of ampicillin and 50 μg/mL of chloramphenicol at 37° C. until optical density (O.D.) reached 0.6 after which the protein production was induced by 0.25 mM isopropyl ß-D-1-thiogalactopyranoside (IPTG) and the cells were grown for 18 hours at 20° C. The cells were harvested by centrifugation at 8000×g for 10 minutes and frozen at −80° C. The frozen pellets were resuspended in the lysis buffer (20 mM Bis Tris pH 7.2, 200 mM NaCl, Roche's complete Protease inhibitors ethylenediaminetetraacetic acid (EDTA)-free, 5 mM tris (2-carboxyethyl) phosphine-HCl (TCEP), 1% Triton™ X-100 and small quantities of DNase and RNase). The resuspended cells were lysed using the One Shot Cell Disrupter (Constant Systems, UK) following which the lysate was mutated for 30 minutes at room temperature. The lysate was centrifuged at 25000×g for 30 minutes to remove cell debris and the supernatant was filtered through a 0.22 m membrane filter (Millipore Sigma, Saint Louis, Missouri, United States of America). Imidazole equivalent to 50 mM final concentration was added to the filtrate containing the MBP-Δ2C-(His)₆ protein. The protein was first purified using the Ni-NTA affinity chromatography (HisTrap Cytiva, Marlborough, Massachusetts, United States of America) and then through Size exclusion chromatography using the Superdex® S200 10/300 column (Cytiva, Marlborough, Massachusetts, United States of America). The affinity chromatography buffer A contained 20 mM Bis Tris pH 7.2, 200 mM NaCl and 0.1% Triton X-100 while the elution buffer contained 500 mM imidazole in addition to buffer A components. Size exclusion chromatography (SEC) buffer contained 20 mM Bis Tris pH 7.2 and 200 mM NaCl.

2C ATPase Assay

The ATPase activity of the MBP-Δ2C-(His)₆ was confirmed using the colorimetric Malachite green assay. This assay was performed in 384-well plates. Each 50 μL reaction contained 2 μM of Δ2C protein, a fixed concentration of the compound or DMSO, 0.5 μM ATP, 5 mM MgCl2 and 20 mM Bis Tris pH 7.2 and was incubated at room temperature for 18 hours before adding 100 μL of the color reagent (0.045% malachite green (Fisher Scientific, Waltham, Massachusetts, United States of America), 34 mM ammonium molybdate (Fisher Scientific, Waltham, Massachusetts, United States of America) in 4 N HCl and 0.02% Tween-20 (Fisher Scientific, Waltham, Massachusetts, United States of America). The plate was then incubated at room temperature for 15 minutes before reading the absorbance at 660 nm. The inhibitory effects of the small molecule compounds were first measured at 50 μM after which a dose response curve was generated for the hits to calculate their IC₅₀ values. The DMSO concentration was kept constant at 10% after ensuring that the ATPase activity of 2C was not inhibited by 10% DMSO. GnHCl and the racemic mixture of Fluoxetine were tested as positive controls and so the appropriate IC₅₀ value was used as a reference.

For the dose-response curves, percent inhibition of ATPase activity was calculated according to the following formula and plotted against the drug concentration:

$$\% inhibition = [ 1 - ( \frac{Drug treated 2 C - Buffer Control}{DMSO treated 2 C - Buffer control} ) ] * 100$$

Curve fitting was carried out in GraphPad Prism using non-linear regression analysis and the IC₅₀ was calculated using the log(inhibitor) vs. respo-se—Variable slope model. The assay was performed three times from which the 95% confidence interval range for IC₅₀ was calculated using profile likelihood asymmetrical confidence intervals. The bottom constraint was set as Zero and the top constraint was set as 100 only in cases where a plateau was not observed at higher compound concentrations.

Differential Scanning Fluorimetry (DSF)

The binding of the compounds to 2C was assessed by studying the thermal stability of 2C by DSF using Sypro Orange. This was monitored using Bio-Rad CFX96 Touch Real-Time PCR Detection System using the fluorescence resonance energy transfer (FRET) channel. Each 50 L reaction contained 2 μM of Δ2C protein and a fixed concentration of the compound that was incubated at room temperature for 30 minutes. Following incubation, Sypro Orange at 5× concentration was added to the reaction and the plate was read for fluorescence. Final DMSO concentration was 4% before the addition of Sypro Orange. The compounds were tested at six different concentrations (200 μM, 40 μM, 8 μM, 1.6 μM, 0.32 μM and 0.02 μM) to study the dose-dependent shift in the melting temperature (Tₘ) of 2C upon interaction with the compound.

Molecular Docking

The molecular docking studies were carried out using the Schrödinger suite (v. 2022-1). First, the compound structures were downloaded as .sdf from PubChem. In the Maestro application, the ligands were prepped using the LigPrep application. The 2C protein structure (PDB: 5GRB, Chain F) was loaded and prepped using the default settings in the Protein Preparation wizard including H-bond optimization and energy minimizations. Docking experiments were performed using the Induced Fit Docking (extended sampling) application in Maestro. Once the docked poses were generated, the top 10 poses were selected to generate the common residue interaction profile using the Interaction Fingerprint application.

Similarities Study

All the computational studies were performed using either Maestro, the graphical user interface for Schrodinger Suite (2021) or Canvas. The three-dimensional structure of each molecule was generated using the ‘build’ module within Schrodinger. Hashed binary fingerprints of each compound were generated by linear fragments with the ring closure method. Using the binary fingerprints as the geometric atomic descriptor, the similarity study was carried out using Tanimoto association coefficient, which gives the similarity values in scale from 0 to 1. The Tanimoto distance metric is a normalized measure of the similarity in descriptor space between a series of test compounds and a probe molecule. Similarities lie between one and zero with a value of one indicating identical molecules and a value of zero indicating completely dissimilar molecules. Further hierarchical clustering was performed using the binary fingerprints generated for the compounds.

The Tanimoto distance metrics were calculated as follows:

$$\frac{{\sum }_{i} {x}_{i} {x}_{i}^{probe}}{{\sum }_{i} {x}_{i} {x}_{i} + {\sum }_{i} {x}_{i}^{probe} {x}_{i}^{probe} - {\sum }_{i} {x}_{i} {x}_{i}^{probe}}$$

In this equation, xᵢ=values of data point i in test molecules, xᵢ^{Probe}=values of data point i in probe molecule 1, xᵢxᵢ^{probe}=values of common data point i in both test and probe molecule.

Multiplicity of Infection (MOI) Screening

Multiplicity of infection (MOI) is defined as the number plaque forming units (PFU) of the virus is present per host cell. Screening was performed to determine a MOI that would result in complete cell death after 3 days of infection. This MOI was first determined for each of the virus in their respective cell system. For example, for EV-A71, Vero cells were used. For this assay, the Cell Titer Glo® One Solution Assay from Promega (Madison, Wisconsin, United States of America) was used. Vero cells were seeded in 96-well white plates at a density of 25,000 cells per well and was incubated overnight at 37° C. in a 5% CO2 incubator. Next day, multiple dilutions of the viral stock with a known titer (PFU/mL) is prepared using serum-free DMEM (supplemented with 1× Anti-Anti) such that a range of MOIs can be screened. Highest MOI tested was 10 and a 2× dilution series was prepared. 200 μL of the respective dilution of virus was added per well. The virus was adsorbed for 2 hours in the incubator. After 2 hours, the virus was removed and 250 μL growth media (DMEM+10% FBS+1× Anti-Anti) was added to each well. Control wells containing cells without virus were also included. The plate was incubated for 3 days. After 3 days, the Cell Titer Glo® One Solution Assay (Promega, Madison, Wisconsin, United States of America) was used to measure the cell viability. 150 μL of the media from each well was discarded and 100 μL of the Cell Titer Glo® reagent was added to each well. The plate was carefully shaken for 2 minutes and then was incubated at room temperature in the biosafety cabinet for 10 minutes before reading the luminescence. The luminescence was then plotted against the MOI and the minimum MOI at which all the cells were dead (lowest luminescence) was noted.

CPE Inhibition Assay

Antiviral activity of the compounds on EV-A71, EV-D68, poliovirus and Coxsackievirus B3 was carried out using a cytopathic effect (CPE) inhibition assay. For this assay, the Cell Titer Glo® One solution Assay from Promega was used. Vero cells were seeded in 96-well white plates at a density of 25,000 cells per well and were incubated overnight at 37° C. in a 5% CO₂ incubator. The next day, the cells were infected with EV-A71 at a MOI of 1 and the virus was let to adsorb for 2 hours in the incubator at 37° C. and 5% CO₂. After 2 hours, the virus was discarded, and the compound was added at a fixed concentration of either 50 μM or 25 μM and the plate was incubated for 3 days at 37° C. and 5% CO₂. The compound was dissolved in DMSO and the required concentration was prepared in growth media. Further, the final DMSO concentration in each well was kept constant at 1%. For dose-response curves, instead of one concentration of the compound, a range of concentrations from 0.1 μM to 50 μM, were used. After 3 days, the Cell Titer Glo® One Solution Assay was used to measure the cell viability. 150 μL of the media from each well was discarded and 100 μL of the Cell Titer Glo® reagent was added to each well. The plate was carefully shaken for 2 minutes and then was incubated at room temperature in the biosafety cabinet for 10 minutes before reading the luminescence.

For the IC₅₀ calculations, percent inhibition of cytopathic effect (CPE) was plotted against the drug concentration. Percent CPE was calculated using the following formula:

$$\% Inhibition = \frac{Compound treated Infected Live cells - Infected dead cells}{DMSO treated Uninfected Live cells - Infected dead cells} * 100$$

Curve fitting was carried out in GraphPad Prism using non-linear regression analysis and the IC₅₀ was calculated using the log(inhibitor) vs. response—Variable slope model. Further, the curve fitting was carried out with a bottom constraint of 0 while a top constraint of 100 was added to plots that did not reach a plateau at higher compound concentrations. The assay was performed three times from which the 95% confidence interval range for IC₅₀ was calculated using profile likelihood asymmetrical confidence intervals.

Cytotoxicity Assay

Cellular toxicity of the compounds was tested using the cytotoxicity assay and a dose-response assay was performed to determine the CC₅₀ for each of the compound. For this assay, the Cell Titer Glo® One Solution Assay from Promega was used. Vero cells were seeded in 96-well white plates at a density of 25,000 cells per well and was incubated overnight at 37° C. in a 5% CO₂ incubator. The next day, serum free media was added to the cells and the plate was incubated for 2 hours in the incubator at 37° C. and 5% CO₂. After 2 hours, the media was discarded, and the compound was added, and the plate was incubated for 3 days at 37° C. and 5% CO₂. The compound was dissolved in DMSO and the required concentration was prepared in growth media. Further, the final DMSO concentration in each well was kept constant at 1%. For dose-response curves, a range of concentrations lying within 0.5 μM to 1000 μM were used depending upon the availability of the compounds. After 3 days, the Cell Titer Glo® One Solution Assay was used to measure the cell viability. 150 μL of the media from each well was discarded and 100 μL of the Cell Titer Glo® reagent was added to each well. The plate was carefully shaken for 2 minutes and then was incubated at room temperature in the biosafety cabinet for 10 minutes before reading the luminescence.

The CC₅₀ values were calculated by plotting percent viability against the drug concentration.

$$\% viability = \frac{Drug treated cells}{DMSO treated cells} * 100$$

Curve fitting was carried out in GraphPad Prism using non-linear regression analysis and the IC₅₀ was calculated using the log(inhibitor) vs. response—Variable slope model. Further, the curve fitting was carried out with a bottom constraint of 0 in cases where the compound was not cytotoxic at higher concentrations and percent viability did not reach 0%. The assay was performed three times.

EV-A71 Replicon Assay

HeLa cells were maintained using Dulbecco's modified Eagle's medium (DMEM) containing 10% (v/v) fetal bovine serum (Sigma, Saint Louis, Missouri, United States of America), 1% Glutamax (Thermo Fisher Scientific, Waltham, Massachusetts, United States of America) and penicillin-streptomycin (Sigma, Saint Louis, Missouri, United States of America) (termed complete DMEM or cDMEM).

EV71 replicon plasmid was linearized using XhoI (NEB, Ipswich, Massachusetts, United States of America) prior to in vitro transcription using the T7 RiboMAX system (Promega) following the manufacturer protocol but with all volumes and concentrations reduced by a factor of two. RNA transcripts were recovered using the RNA Clean and Concentrator-25 kit (Zymo Research, Irvine, California, United States of America), with concentrations determined by NanoDrop (Thermo Fisher Scientific, Waltham, Massachusetts, United States of America) and RNA integrity determined by 3-(N-morpholino)propanesulfonic acid (MOPS)-formaldehyde denaturing gel electrophoresis. HeLa cells were seeded 16 hours prior to RNA transfection at a density of 10,000 cells per well of a 96-well plate. WT and 3D-GNN (replication defective mutant) RNAs were prepared by diluting 160 ng/well RNA and 0.48 μL/well Lipofectamine™ 2000 (Thermo Fisher Scientific, Waltham, Massachusetts, United States of America) in opti-MEM™ (Thermo Fisher Scientific, Waltham, Massachusetts, United States of America). Following a 10-minute incubation, RNA and Lipofectamine™ mixes were combined and incubated for a further 20 minutes. Transfection mix was added to cDMEM without phenol-red indicator, alongside chemical compounds where applicable, to a final volume of 100 μL/well. Cells were washed 1× with phosphate-buffered saline (PBS) before addition of RNA transfection/cDMEM mixture to wells in triplicate. Four images of each well were taken hourly using an IncuCyte® S3 Live Cell Imaging System (Sartorius, Göttingen, Germany) to visualize fluorescent reporter protein expression. Fluorescent cells were determined using the inbuilt IncuCyte analysis package using surface fit segmentation and filters to emit any signal with a threshold value (GCU; an arbitrary green fluorescent unit)<10 and surface area <50 μm²; values that were determined using no transfection and 3D-GNN controls to remove background fluorescence. The average number of positive cells per condition 14-hours post transfection across triplicates were compared to untreated WT transfected wells. Averages of three independent repeats were plotted for each sample and statistical analysis was performed using a two-way ANOVA unless otherwise indicated.

Escape Mutant Assay

Escape mutant assay involves selecting resistant viruses to specific anti-virals to determine their mechanism of action. To select escape mutants for Compound 167, Vero cells were seeded at a density of 25,000 cells per well of a 96-well plate and incubated overnight at 37° C. in a 5% CO₂ incubator. The next day, the cells were infected with EV-A71 at a MOI of 5 and the virus was let to adsorb for 2 hours in the incubator at 37° C. and 5% CO₂. After 2 hours, the virus was discarded, and the compound was added at a fixed concentration of 0.5×IC₅₀ value (30 μM) and the plate was incubated for 3 days at 37° C. and 5% CO₂. After 3 days, the cells were checked for cytopathic effects (CPE) using a light microscope. CPE indicated successful viral infection and the supernatant media contained the progeny virus. This virus was termed as generation 1 (Gen 1). The virus from Gen 1 was used to infect a freshly seeded plate of Vero cells. Following the 2-hour adsorption, the virus was removed and saved. Fresh media containing 0.5×IC₅₀ value (30 μM) of Compound 167 was added to the cells and incubated for 3 days at 37° C. and 5% CO2. The supernatant media containing the virus was then used to infect a new batch of Vero cells and the process is repeated until 15 generations of virus were produced including five generations with 0.5×IC₅₀ value (30 μM), followed by five generations with 1×IC₅₀ (60 μM) and finally, five generations with 2×IC₅₀ (120 μM). Escape mutants for Compound 36 was extracted similarly with a few modifications. The 15 generations were produced such that there were three generations at five different drug concentrations—0.5×IC₅₀, 1×IC₅₀, 2×IC₅₀, 4×IC₅₀ and 10×IC₅₀. A similar approach was followed to generate the DMSO treated virus as well to compare the mutations. The DMSO concentration was kept constant at 1% throughout the 15 generations. The viral genome of Gen 15 was isolated using TRIzol™ extraction. The genome was then sequenced using next generation sequencing.

Total RNA Quality Control

Total RNA was quantified, and purity ratios were determined for each sample using the NanoDrop 2000 spectrophotometer (Thermo Fisher Scientific, Waltham, Massachusetts, United States of America). To assess RNA quality, total RNA was analyzed on the Agilent TapeStation 4200 (Agilent Technologies, Santa Clara, California, United States of America) using the RNA High Sensitivity assay following the manufacturer's protocol. Ribosomal Integrity Numbers (RINe) were recorded for each sample.

Illumina Total RNA Library Preparation and Sequencing

Total RNA samples (250 ng of Qubit quantified total RNA input) were prepared for library preparation using the Illumina Stranded Total RNA Gold library preparation kit (Illumina, San Diego, California, United States of America) following the manufacturer's protocol. Libraries were validated for length and adapter dimer removal using the Agilent TapeStation 4200 D1000 High Sensitivity assay (Agilent Technologies, Santa Clara, California, United States of America) then quantified and normalized using the dsDNA High Sensitivity Assay for Qubit 3.0 (Life Technologies, Carlsbad, California, United States of America).

Sample libraries were prepared for Illumina sequencing on the NovaSeq 6000 by denaturing and diluting the libraries per the manufacturer's protocol (Illumina, San Diego, California, United States of America). All samples were combined into one sequencing pool, proportioned according to the expected number of reads, and run as one sample pool. Target read depth was achieved per sample with paired-end 150 basepair (bp) reads.

Plaque Assay for Viral Titer Determination

To compare the resistance of the escape mutant with wild-type, sufficient wild-type (WT) and T237N mutant virus stock that would result in complete cell death after 3 days post-infection was used as the starting virus concentration. Six serial dilutions were carried out of this virus and used in plaque assay as described here to determine the viral titer in the presence of 500 μM of Compound 167. Vero cells, cultured in growth media (DMEM+10% FBS+1× Anti-Anti), were seeded at a density of 500,000 cells/well in a clear 6-well plate and incubated overnight at 37° C. in a 5% CO₂ incubator. The next day, six 10-fold serial dilution of the virus was prepared in serum-free DMEM (with 1× Anti-Anti). 500 μL from of the dilution was added to the cells and the plate was incubated at 37° C. in a 5% CO₂ incubator. The plate was rocked gently every 20 minutes to ensure even coverage and prevent the cellular monolayer from drying. After 2 hours adsorption, the virus was removed, and the cells were washed with phosphate-buffered saline (PBS). 2 mL of the liquid overlay was added to each well and the plate was incubated at 37° C. in a 5% CO₂ incubator for 3 days. The liquid overlay was prepared by mixing 1:1 2.4% Avicel (in PBS) and growth media so that the final concentration of Avicel was 1.2%. When testing the effect of Compound 167 on the viral titer, the compound was added to the liquid overlay at a final concentration of 500 μM which is slightly lower than 10× its IC₅₀ (59.4 μM). After 3 days the cells were fixed and stained. To fix the cells, 1 mL of 4% formaldehyde was added to each well and incubated at room temperature in the biosafety cabinet for 30 minutes. Then the formaldehyde-Avicel overlay was poured out and washed with de-ionized water. 1 mL of 1% crystal violet staining solution was then added to each well. After 1 hour the stain was discarded and the wells rinsed in water first and then in 0.5% bleach. The plates were dried in the biosafety cabinet overnight. Once the plate was dry, the viral titer was determined by the following formula—

$$PFU / mL = \frac{Average number of plaques}{Dilution of virus * Volume of diluted virus added to each well}$$

Example—If there were 32 plaques in the well which was infected with 500 mL of the 10⁻⁵ virus dilution, then the viral titer would be 64×10⁻⁵ PFU/mL [32 plaques/(10⁻⁵*0.5 mL)].

### Definitions

The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted. The opened ended term “comprising” includes the intermediate and closed terms “consisting essentially of” and “consisting of” Only the transitional phrases “consisting of” and “consisting essentially of” shall be closed or semi-closed transitional phrases, respectively, as set forth in the United States Patent Office Manual of Patent Examining Procedures, Section 2111.03.

It should also be understood that, in certain methods or processes described herein that include more than one step or act, the order of the steps or acts of the method is not necessarily limited to the order in which the steps or acts of the method are recited unless the context indicates otherwise.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” or “providing” means the actual physical introduction of a composition into or onto (as appropriate) a subject, a host or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. It also means giving, administering, selling, distributing, transferring (for profit or not), manufacturing, compounding, or dispensing

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

As used herein, the term “effective amount” or “therapeutically effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

Effective amounts may vary depending upon the biological effect desired in the individual, condition to be treated, and/or the specific characteristics of the composition according to the present invention and the individual. In this respect, any suitable dose of the composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the composition according to the present invention desirably comprises about 0.1 mg per kilogram (kg) of the body weight of the patient (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject is a human, such as a human being treated or assessed for a disease, disorder or condition, a human at risk for a disease, disorder or condition, a human having a disease, disorder or condition, and/or human being treated for a disease, disorder or condition as described herein. In some embodiments, the subject does not suffer from an ongoing autoimmune disease. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included.

The term “substituted” means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Suitable groups that may be present on an “optionally substituted” position include, but are not limited to, e.g., halogen, cyano, hydroxyl, amino, nitro, oxo, azido, alkanoyl (such as a C₂-C₆ alkanoyl group such as acyl or the like (—(C═O)alkyl)); carboxamido; alkylcarboxamide; alkyl groups, alkoxy groups, alkylthio groups including those having one or more thioether linkages, alkylsulfinyl groups including those having one or more sulfinyl linkages, alkylsulfonyl groups including those having one or more sulfonyl linkages, mono- and di-aminoalkyl groups including groups having one or more N atoms, all of the foregoing optional alkyl substituents may have one or more methylene groups replaced by an oxygen or —NH—, and have from about 1 to about 8, from about 1 to about 6, or from 1 to about 4 carbon atoms, cycloalkyl; phenyl; phenylalkyl with benzyl being an exemplary phenylalkyl group, phenylalkoxy with benzyloxy being an exemplary phenylalkoxy group. Alkylthio and alkoxy groups are attached to the position they substitute by the sulfur or oxygen atom respectively.

A dash (“-”) that is not between two letters or symbols is used to indicate a point of attachment for a substituent.

An “aliphatic group” is a non-aromatic hydrocarbon group having the indicated number of carbon atoms. Aliphatic groups may be saturated, unsaturated, or cyclic.

“Alkyl” includes both branched and straight chain saturated aliphatic hydrocarbon groups, having the specified number of carbon atoms, generally from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g., C₁-C₈alkyl, C₁-C₄alkyl, and C₁-C₂alkyl. When C₀-Cₙ alkyl is used herein in conjunction with another group, for example, —C₀-C₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, 3-methylbutyl, t-butyl, n-pentyl, and sec-pentyl.

“Alkenyl” is a branched or straight chain aliphatic hydrocarbon group having one or more carbon-carbon double bonds that may occur at any stable point along the chain, having the specified number of carbon atoms. Examples of alkenyl include, but are not limited to, ethenyl and propenyl.

“Alkylene” when used, refers to a —(CH₂)ₙ— group (n is an integer generally from 0-6), which may be optionally substituted. When substituted, the alkylene group preferably is substituted on one or more of the methylene groups with a C₁-C₆ alkyl group (including a cyclopropyl group or a t-butyl group), but may also be substituted with one or more halo groups, preferably from 1 to 3 halo groups or one or two hydroxyl groups, O—(C₁-C₆ alkyl) groups or amino acid sidechains as otherwise disclosed herein.

“Alkynyl” is a branched or straight chain aliphatic hydrocarbon group having one or more double carbon-carbon triple bonds that may occur at any stable point along the chain, having the specified number of carbon atoms.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “Alkylthio” or a “thioalkyl” group is an alkyl group as defined above with the indicated number of carbon atoms covalently bound to the group it substitutes by a sulfur bridge (—S—).

“Aryl” indicates aromatic groups containing only carbon in the aromatic ring or rings. Typical aryl groups contain 1 to 3 separate, fused, or pendant rings and from 6 to about 18 ring atoms, without heteroatoms as ring members. It includes mono-, bi-, tri-, or polycyclic aryl group. When indicated, such aryl groups may be further substituted with carbon or non-carbon atoms or groups. Aryl groups include, for example, phenyl, naphthyl, including 1-naphthyl, 2-naphthyl, and bi-phenyl.

The term “acyl” is a term of art and as used herein refers to any group or radical of the form RCO- where R is any organic group, e.g., alkyl, aryl, heteroaryl, aralkyl, and heteroaralkyl. Representative acyl groups include acetyl, benzoyl, and malonyl.

The term “amino” is a term of art and as used herein refers to both unsubstituted and substituted amines, e.g., a moiety that may be represented by the general formulas:

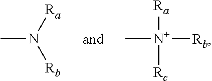

wherein Rₐ, R_{b}, and R_{c} each independently represent a hydrogen, an alkyl, an alkenyl, —(CH₂)ₓ—R_{d}, or Rₐ and R_{b}, taken together with the N atom to which they are attached complete a heterocycle having from 4 to 8 atoms in the ring structure; R_{d} represents an aryl, a cycloalkyl, a cycloalkenyl, a heterocyclyl or a polycyclyl; and x is zero or an integer in the range of 1 to 8. In certain embodiments, only one of Rₐ or R_{b} may be a carbonyl, e.g., Rₐ, R_{b}, and the nitrogen together do not form an imide. In other embodiments, Rₐ and R_{b} (and optionally R_{c}) each independently represent a hydrogen, an alkyl, an alkenyl, or —(CH₂)ₓ—R_{d}. In certain embodiments, the term “amino” refers to —NH₂.

The term “carboxamido”, as used herein, means —NHC(═O)—, wherein the carboxamido group is bound to the parent molecular moiety through the nitrogen. Examples of carboxamido include alkylcarboxamido such as CH₃C(═O)N(H)— and CH₃CH₂C(═O)N(H)—.

“Cycloalkyl” is a saturated hydrocarbon ring group, having the specified number of carbon atoms, usually from 3 to about 7 carbon atoms. Examples of cycloalkyl groups include cyclopropyl, cyclobutyl, cyclopentyl, or cyclohexyl as well as bridged or caged saturated ring groups such as norbornane or adamantane. “—(C₀-Cₙalkyl)cycloalkyl” is a cycloalkyl group attached to the position it substitutes either by a single covalent bond (Co) or by an alkylene linker having 1 to n carbon atoms. In the term “(cycloalkyl)alkyl,” cycloalkyl and alkyl are as defined above, and the point of attachment in on the alkyl group.

“Halo” or “halogen” means fluoro, chloro, bromo, or iodo.

“Heteroaryl” is a stable monocyclic aromatic ring having the indicated number of ring atoms which contains from 1 to 3, or in some embodiments from 1 to 2, heteroatoms chosen from N, O, and S, with remaining ring atoms being carbon, or a stable bicyclic or tricyclic system containing at least one 5- to 7-membered aromatic ring which contains from 1 to 3, or in some embodiments from 1 to 2, heteroatoms chosen from N, O, and S, with remaining ring atoms being carbon. Monocyclic heteroaryl groups typically have from 5 to 7 ring atoms. In some embodiments bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the aromatic heterocycle is not more than 1. Heteroaryl groups include, but are not limited to, oxazolyl, piperazinyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxinyl, furanyl, imidazolyl, indolyl, isothiazolyl, and isoxazolyl.

“Heterocycle” is a saturated, unsaturated, or aromatic cyclic group having the indicated number of ring atoms containing from 1 to about 3 heteroatoms chosen from N, O, and S, with remaining ring atoms being carbon. Examples of heterocycle groups include piperazine and thiazole groups.

“Heterocycloalkyl” is a saturated cyclic group having the indicated number of ring atoms containing from 1 to about 3 heteroatoms chosen from N, O, and S, with remaining ring atoms being carbon. Examples of heterocycloalkyl groups include tetrahydrofuranyl, morpholinyl, and pyrrolidinyl groups.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Hydrocarbyl” shall mean a compound which contains carbon and hydrogen and which may be fully saturated, partially unsaturated or aromatic and includes aryl groups, alkyl groups, alkenyl groups and alkynyl groups.

“Pharmaceutical compositions” means compositions comprising at least one active agent, such as a compound or salt of Formula (I), and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and 1311.

A significant change is any detectable change that is statistically significant in a standard parametric test of statistical significance such as Student's T-test, where p<0.05.

The composition according to the present invention may be administered to a patient by various routes. Examples of routes of administration include, but are not limited to, parenteral, e.g., intravenous, intradermal, subcutaneous, oral, intranasal (e.g., inhalation), transdermal (e.g., topical), transmucosal, and rectal administration. In an embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer.

In accordance with any of the embodiments, the composition according to the present invention can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The dose administered to the mammal, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular composition and the desired effect. It will be appreciated by one of skill in the art that various conditions or disease states may require prolonged treatment involving multiple administrations.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating or preventing a disease or disorder, or a symptom associated with a picornavirus infection in a subject, the method comprising:
providing and administering a therapeutically effective amount of a compound of Formula I, or a pharmaceutically acceptable salt thereof, or a composition comprising the same to the subject, wherein Formula I has the chemical structure: 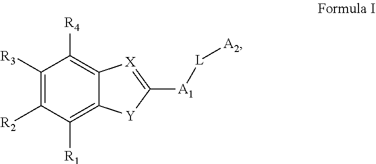
wherein in Formula I,
X is CR₅ or N;
Y is CR₅R₆, NR₅, O, or S;
L is a linker comprising O, NR₅, CONR₅, COO, COS, or S;
A₁ and A₂ are each independently an aryl (such as a monocyclic aryl or bicyclic aryl), a heteroaryl (such as a monoheteroaryl or bicyclic heteroaryl), an aryloxy (such as phenoxy), a thioaryl (such as thiophenyl), an alkyl (such as C₁-C₆ alkyl), an alkylene (such as a C₂₋₆ alkylene), an alkenyl (such as a C₂₋₆ alkenyl, ethenyl, or propenyl), an alkynyl (such as a C₂₋₆ alkynyl or acetylenyl), a haloalkyl (such as C₁-C₆ haloalkyl), an alkoxy (such as C₁-C₆ alkoxy), a cycloalkyl (such as a C₃-C₈ cycloalkyl group), a heterocycloalkyl (such as a 5-8 membered heterocycloalkyl), an alkylaryl (such as a C₁₋₃ alkyl-C₈₋₁₁ aryl, C₁₋₃ alkyl-C₆ aryl, C₁₋₃ alkyl-C₈ aryl, or C₁₋₃ alkyl-C₁₁ aryl), an alkylheteroaryl (such as a C₁₋₃ alkyl-5-11 membered heteroaryl or C₁₋₃ alkyl-5-8 membered heteroaryl), or a (cycloalkyl)alkyl (such as C₁₋₃ alkyl-C₃₋₈ cycloalkyl),
wherein A₁ and A₂ are each optionally substituted with one or more (such as 1, 2, 3, 4, or more) substituents independently selected from halogen, hydroxyl, cyano, nitro, amino, alkyl (such as C₁-C₆alkyl), alkoxy (such as C₁-C₄ alkoxy or methoxy), cycloalkyl (such as C₃-C₈ Cycloalkyl), haloalkyl (such as C₁-C₂haloalkyl), haloalkoxy (such as C₁-C₂haloalkoxy), aryl (such as a C₆ or C₈ aryl), heteroaryl (such as a 5-8 membered heteroaryl), heterocycloalkyl (such as a 3-8 or 5-8 membered heterocycloalkyl), an aryloxy (such as C₆ or C₈ aryloxy or phenoxy), a thioaryl (such as C₆ or C₈ thioaryl thiophenyl), —CHO, —COOH, —CONR₅R₆, —COR₅, —NO₂, —O—CO—R₆, —NR₅—CO—R₆, and hydrocarbyl having a hydrocarbon chain of 1 to 6 carbon atoms in which the carbon atoms are joined by single, double or triple bonds, and any one carbon atom or any two nonadjacent carbon atoms can be replaced by O, NH, N(C₁-C₄ alkyl), or S, wherein the hydrocarbyl group is optionally substituted with one or more substituents independently chosen from hydroxyl, oxo, halogen, and amino; and
R₁, R₂, R₃, R₄, R₅, and R₆ are each independently hydrogen, halogen, hydroxyl, cyano, nitro, amino, —CHO, —CONH₂, —CO-aryl (such as benzoyl), —CO-alkyl (such as acetyl), alkyl (such as C₁-C₆ alkyl), alkoxy (such as C₁-C₄alkoxy), cycloalkyl (such as C₃-C₈ cycloalkyl), haloalkyl (such as C₁-C₂haloalkyl), haloalkoxy (such as C₁-C₂haloalkoxy), aryl (such as a C₆₋₁₁ aryl, a C₆, C₈ or C₁₁ aryl, phenyl, naphthyl, or tolyl), heteroaryl (such as 5-8 membered heteroaryl, furyl, or benzofuryl), heterocycloalkyl (such as a 3-8 membered heterocycloalkyl), an aryloxy (such as phenoxy), alkylaryl (such as —CH₂—CH₂-Ph), or a thioaryl (such as thiophenyl);
wherein two adjacent R groups, R₁ and R₂, R₂ and R₃, or R₃ and R₄ are optionally combined together with the carbon atoms they are attached to form a 6- or 8-membered aromatic ring or a 5-8 membered heteroaromatic ring,
wherein A₂ is optionally substituted by one substituent having the structure —CONR₅ₐR₆ₐ(such as —CONHR₆ₐ), wherein R₅ₐ is hydrogen or a C₁₋₃ alkyl, and R₆ₐ is a bicyclic heteroaryl (such as a 9-11 or 10 membered bicyclic heteroaryl) or a C₆ or C₈ aryl substituted with a bicyclic heteroaryl (such as a 9-11 or 10 membered bicyclic heteroaryl), and
wherein the method is effective in treating, preventing, or ameliorating the disease or disorder associated with a picornavirus infection, or at least one symptom of the disease or disorder associated with a picornavirus infection.

2. The method of claim 1, wherein:
A₁ is an monocyclic aryl (such as a C₆ or C₈ aryl), a monocyclic heteroaryl (such as a 5-8 membered heteroaryl having 1, 2, or 3 heteroatoms independently selected from N, O, and S), a mono cycloalkyl (such as a C₃₋₈ cycloalkyl), or a mono heterocycloalkyl (such as a 5-8 membered heterocycloalkyl having 1, 2, or 3 heteroatoms independently selected from N, O, and S);
A₂ is an alkyl (such as C₁₋₆ alkyl) optionally having a heteroatom (such as NH, O, or S) replacing one of the carbon atoms, alkylene (such as such as a C₂₋₆ alkylene), alkenyl (such as a C₂₋₆ alkenyl, ethenyl, or propenyl), alkynyl (such as a C₂₋₆ alkynyl or acetylenyl), a haloalkyl (such as C₁₋₆haloalkyl), cycloalkyl (such as a C₃₋₈ cycloalkyl group), monocyclic aryl (such as phenyl), —CH₂-thioaryl (such as a C₆ or C₈ thioaryl), bicyclic aryl (such as a monocyclic aryl or bicyclic aryl), monocyclic heteroaryl (such as a 5-8 membered heteroaryl having 1, 2, or 3 heteroatoms independently selected from N, O, and S), aryloxy (such as a C₆ or C₈ aryloxy or phenoxy), thioaryl (such as thiophenyl), a bicyclic heteroaryl (such as a 9-11 membered heteroaryl having 1, 2, 3, 4, or 5 heteroatoms independently selected from N, O, and S), alkoxy (such as C₁₋₆ alkoxy), alkylaryl (such as a C₁₋₃ alkyl-C₆₋₁₁ aryl, C₁₋₃ alkyl-C₆ aryl, C₁₋₃ alkyl-C₈ aryl or C₁₋₃ alkyl-C₁₁ aryl), alkylheteroaryl (such as a C₁₋₃ alkyl-5-8 membered heteroaryl), (cycloalkyl)alkyl (such as C₁₋₃ alkyl-C₃₋₈ cycloalkyl), (heterocycloalkyl)alkyl (such as C₁₋₃ alkyl-3-8 membered cycloalkyl);
R₁, R₂, R₃, and R₄ are each independently hydrogen, halogen (such as Cl, F, I, or Br), C₁₋₆ alkyl (such as C₁₋₃ alkyl or methyl), hydroxyl, cyano, nitro, amino, —CHO, —CONH₂, —CO-aryl (such as benzoyl), —CO—C₁₋₆ alkyl (such as acetyl), C₁₋₄ alkoxy), C₃₋₈ cycloalkyl), haloalkyl (such as C₁-C₂haloalkyl), haloalkoxy (such as C₁-C₂haloalkoxy), aryl (such as a C₆, C₈ of C₁₁ aryl, phenyl, naphthyl, or tolyl), 5-8 membered heteroaryl (such as a 5- or 6-membered heteroaryl), 3-8 membered heterocycloalkyl (such as a 5- or 6-membered heterocycloalkyl), C₆ or C₈ aryloxy (such as phenoxy), C₁₋₃ alkyl-C₆ or C₈ aryl (such as —CH₂—CH₂-phenyl), or a thioaryl (such as thiophenyl); or
a combination thereof,
wherein:
when A₂ is an alkyl, alkylene, alkenyl or alkynyl, (i) R₂ is an alkyl (such as methyl, ethyl, or propyl), (ii) R₂ and R₃ combined together with the carbon atoms they are attached to form a C₆ aryl, C₈ aryl, or 5-8 membered heteroaryl, or (iii) A₂ is substituted by an aryl (such as a C₆ or C₈ aryl), aryloxy (such as a C₆ or C₈ aryloxy), or a thioaryl (such as a C₆ or C₈ thioaryl), each of which are optionally substituted with one or more (such as 1, 2, 3, 4, or more) substituted independently selected from halogen (such as Cl, Br, I, or F), C₁₋₃ alkyl (such as methyl);
A₂ is optionally substituted by one —CONR₅R₆ₐ or one or more (such as 1, 2, 3, 4, or more) substituent independently selected from halogen (such as Br or Cl), oxo, hydroxyl, C₁₋₃ alkyl, a C₁₋₃ alkoxy (such as methoxy), —CHO, —COOH, —CONR₅R₆, —COR₅, —NO₂, —O—CO—R₅, —NR₅—CO—R₆, C₃₋₈ membered cycloalkyl, a 3-8 membered heterocycloalkyl having 1, 2 or 3 heteroatoms independently selected from N, O and S, C₆ or C₈ aryl, and 5-8 membered heteroaryl having 1, 2 or 3 heteroatoms independently selected from N, O and S;
R₆ₐ is a bicyclic heteroaryl (such as a 9-11 or 10 membered bicyclic heteroaryl) or a C₆ or C₈ aryl substituted with a bicyclic heteroaryl (such as a 9-11 or 10 membered bicyclic heteroaryl);
each R₅ is independently H or —CH₃, wherein each cyclic substituent is optionally substituted with one or more (such as 1, 2, 3, 4, or more) substituted independently selected from halogen (such as Cl, Br, I, or F), C₁₋₃ alkyl (such as methyl), and —NO₂; and
each R₆ is independently H or —CH₃, wherein each cyclic substituent is optionally substituted with one or more (such as 1, 2, 3, 4, or more) substituted independently selected from halogen (such as Cl, Br, I, or F), C₁₋₃ alkyl (such as methyl), and —NO₂.

3. The method of claim 2, wherein:
A₁ is a 6-membered aryl or heteroaryl;
L is CONH;
R₁ and R₄ are hydrogen;
R₂ is a hydrogen or a C₁₋₃ alkyl and R₃ is hydrogen, or R₂ and R₃ are combined together with the carbon atoms they are attached to form a C₆ aryl;
X is N; or
a combination thereof.

4. The method of claim 2, wherein A₁ is a 6-membered aryl or heteroaryl with the chemical structure 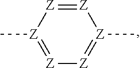
wherein each Z is independently a CH or N, wherein there 0, 1, 2, 3, or 4 N groups.

5. The method of claim 2, wherein:
A₁ is a 6-8 membered monocyclic aromatic group comprising 1, 2, or 3 heteroatoms selected from O, S, and N (e.g., C₆ aryl, C₈ aryl, or a 5-8 membered heteroaryl, such as, 5-7 membered heteroaryl or a 6-membered heteroaryl);
A₂ is a C₁₋₆ alkyl, cycloalkyl (e.g., C₃₋₈ cycloalkyl), C₆₋₁₁ aryl, or 5-8 membered heteroaryl, wherein A₂ is optionally substituted by one or more (such as 1, 2, 3, 4, or more) substituent independently selected from halogen (such as Br or Cl), oxo, hydroxyl, C₁₋₃ alkyl, a C₁₋₃ alkoxy (such as methoxy), —CHO, —COOH, —CONR₅R₆, —COR₅, —NO₂, —O—CO—R₅, —NR₅—CO—R₆, C₃₋₈ membered cycloalkyl, a 3-8 membered heterocycloalkyl having 1, 2 or 3 heteroatoms independently selected from N, O and S, C₆ or C₈ aryl, and 5-8 membered heteroaryl having 1, 2 or 3 heteroatoms independently selected from N, O and S;
L is CONR₅, wherein R₅ is a H or C₁₋₃ alkyl;
R₃ is hydrogen and R₂ is a hydrogen, halogen, or a C₁₋₃ alkyl, or R₂ and R₃ are combined together with the carbon atoms they are attached to form a 6-membered aryl or heteroaryl;
X is N or CH;
R₁ and R₄ are each H; or
a combination thereof.

6. The method of claim 5, wherein:
A₁ is a 6-membered aryl or heteroaryl;
L is CONH;
R₁ and R₄ are hydrogen;
R₂ is a hydrogen or a C₁₋₃ alkyl and R₃ is hydrogen, or R₂ and R₃ are combined together with the carbon atoms they are attached to form a C₆ aryl;
X is N;
a combination thereof.

7. The method of claim 6, wherein A₁ is a 6-membered aryl or heteroaryl with the chemical structure 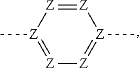
wherein each Z is independently a CH or N, wherein there 0, 1, 2, 3, or 4 N groups.

8. The method of claim 1, wherein the compound is selected from compounds 1-50.

9. A method of treating or preventing a disease or disorder, or a symptom associated with a picornavirus infection in a subject, the method comprising:
providing and administering a therapeutically effective amount of a compound of Formula II, or a pharmaceutically acceptable salt thereof, or a composition comprising the same to the subject, wherein Formula II has the chemical structure: 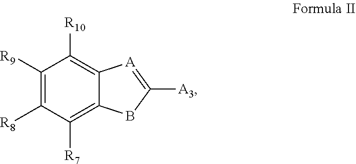
wherein in Formula II,
A is CR₁₁ or N;
B is CR₁₁R₁₂, NR₁₁, O, or S;
A₃ is an aryl (such as a monocyclic aryl or bicyclic aryl), a heteroaryl (such as a monocyclic heteroaryl or bicyclic heteroaryl), an aryloxy (such as phenoxy), a thioaryl (such as thiophenyl), an alkyl (such as C₁-C₆ alkyl), an alkylene (such as a C₂₋₆ alkylene), an alkenyl (such as a C₂₋₆ alkenyl, ethenyl, or propenyl), an alkynyl (such as a C₂₋₆ alkynyl or acetylenyl), a haloalkyl (such as C₁-C₆ haloalkyl), an alkoxy (such as C₁-C₆ alkoxy), a cycloalkyl (such as a C₃-C₈ cycloalkyl group), a heterocycloalkyl (such as a 5-8 membered heterocycloalkyl), an alkylaryl (such as a C₁₋₃ alkyl-C₈₋₁₁ aryl, C₁₋₃ alkyl-C₆ aryl or C₁₋₃ alkyl-C₈ aryl or C₁₋₃ alkyl-C₁₁ aryl), an alkylheteroaryl (such as a C₁₋₃ alkyl-5-11 membered heteroaryl or C₁₋₃ alkyl-5-8 membered heteroaryl), or a (cycloalkyl)alkyl (such as C₁₋₃ alkyl-C₃₋₈ cycloalkyl),
wherein A₃ is optionally substituted with one or more (such as 1, 2, 3, or more) substituents independently selected from halogen (such as Br, F, I, or Cl), hydroxyl, cyano, nitro, amino (such as —NH₂), alkyl (such as C₁-C₆ alkyl or methyl), alkoxy (such as C₁-C₄ alkoxy or methoxy), cycloalkyl (such as C₃-C₈ cycloalkyl), haloalkyl (such as C₁-C₂haloalkyl), haloalkoxy (such as C₁-C₂haloalkoxy), aryl (such as a C₆ or C₈ aryl), heteroaryl (such as a 5-8 membered heteroaryl), heterocycloalkyl (such as a 3-8 or 5-8 membered heterocycloalkyl), an aryloxy (such as C₆ or C₈ aryloxy or phenoxy), a thioaryl (such as C₆ or C₈ thioaryl or thiophenyl), —CHO, —COOH, —CONR₁₁R₁₂, —COR₁₁, —NR₁₁—CO—R₁₂, —NR₁₁ₐCO—R₁₂ₐ (such as —NH—CO—R₁₂ₐ), and hydrocarbyl having a hydrocarbon chain of 1 to 6 carbon atoms in which the carbon atoms are joined by single, double or triple bonds, and any one carbon atom or any two nonadjacent carbon atoms can be replaced by O, NH, N(C₁-C₄ alkyl), or S, and wherein the hydrocarbyl group is optionally substituted with one or more substituents independently chosen from hydroxyl, oxo, halogen, and amino;
R₁₁ₐ is a hydrogen or a C₁₋₃ alkyl;
R₁₂ₐ is a monocyclic heteroaryl (such as a 5-8, 5, 6, or 7 membered heteroaryl), a bicyclic heteroaryl (such as a 9-11 or 10 membered bicyclic heteroaryl), a C₆ or C₈ monocyclic aryl, or a bicyclic aryl (such as a C₉₋₁₁ or C₁₀ bicyclic aryl), wherein the R₁₄ₐ is optionally substituted with one or more (such as 1, 2, or 3) substituents independently selected from halogen (such as Cl, Br, I, or F), C₁₋₃ alkyl (such as methyl), and C₁₋₃ alkoxy (such as methoxy);
R₇, R₈, R₉, R₁₀, R₁₁, and R₁₂ are each independently hydrogen, halogen, hydroxyl, cyano, nitro, amino, —CHO, —CONH₂, —NR₁₃—CO—R₁₄, —CO-aryl (such as benzoyl), —CO-alkyl (such as acetyl), alkyl (such as C₁-C₆ alkyl or methyl), alkoxy (such as C₁-C₄ alkoxy), cycloalkyl (such as C₃-C₈ cycloalkyl), haloalkyl (such as C₁-C₂haloalkyl), haloalkoxy (such as C₁-C₂haloalkoxy), aryl (such as a C₆₋₁₁ aryl, a C₆, C₈ or C₁₁ aryl, phenyl, naphthyl, or tolyl), heteroaryl (such as 5-11, 5, 6 or 10 membered heteroaryl, furyl or benzofuryl), heterocycloalkyl (such as a 3-8 membered heterocycloalkyl), an aryloxy (such as phenoxy), alkylaryl (such as —CH₂—CH₂-phenyl), or a thioaryl (such as thiophenyl);
R₁₃ and R₁₄ are each independently hydrogen, halogen, hydroxyl, cyano, nitro, amino, —CHO, —CONH₂, —CO-aryl (such as benzoyl), —CO-heteroaryl (such as 5-8, 5 or 6 membered heteroaryl), —CO-alkyl (such as acetyl), alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), haloalkoxy (such as C₁-C₂haloalkoxy), aryl (such as phenyl, naphthyl, or tolyl), heteroaryl (such as furyl, or benzofuryl), heterocycloalkyl (such as 5-8, 5, or 6 membered heterocycloalkyl), an aryloxy (such as phenoxy), alkylaryl (such as —CH₂—CH₂-phenyl), or a thioaryl (such as thiophenyl), wherein R₁₄ is optionally substituted with one or more (such as 1, 2, or 3) substituents independently selected from halogen (such as Cl, Br, I, or F), C₁₋₃ alkyl (such as methyl), and C₁₋₃ alkoxy (such as methoxy;
wherein two adjacent R groups, R₇ and R₈, R₈ and R₉, or R₉ and R₁₀ are optionally combined together with the carbon atoms they are attached to form a 6- or 8-membered aromatic ring or 5-8 membered heteroaromatic ring;
wherein the method is effective in treating, preventing, or ameliorating the disease or disorder associated with a picornavirus infection, or at least one symptom of the disease or disorder associated with a picornavirus infection.

10. The method of claim 9, wherein
A₃ is a C₆ aryl or 6-membered heteroaryl;
A₃ is optionally substituted with 1 or 2 substituents independently selected from halogen (such as Br, F, I, or Cl), —NR₁₁ₐ—CO—R₁₂ₐ (such as —NH—CO—R₁₂ₐ), hydroxyl, cyano, nitro, amino (such as —NH₂), C₁₋₃ alkyl (such as methyl), C₁₋₄ alkoxy (such as methoxy), C₃₋₈ cycloalkyl, C₁₋₂ haloalkyl, C₁₋₂haloalkoxy, C₆ aryl, C₈ aryl, 5-8 membered heteroaryl, 3-8 membered heterocycloalkyl (such as 5-8 membered heterocycloalkyl), —CHO, and —COOH;
R₁₁ₐ is a hydrogen or a C₁₋₃ alkyl;
R₁₂ₐ is a 5-8 membered heteroaryl (such as 5, 6, or 7 membered heteroaryl), 9-11 membered bicyclic heteroaryl (such as 10 membered bicyclic heteroaryl), C₆ monocyclic aryl, C₈ monocyclic aryl, or C₉₋₁₁ bicyclic aryl (such as C₁₀ bicyclic aryl), wherein the R₁₄ₐ is optionally substituted with 1, 2, or 3 substituents independently selected from halogen (such as Cl, Br, I, or F), or C₁₋₃ alkyl (such as methyl), and C₁₋₃ alkoxy (such as methoxy);
R₇, R₉, and R₁₀ are each hydrogen;
A is N;
B is NR₁₁;
R₁₁ is hydrogen or C₁₋₆ alkyl (such as C₁₋₃ alkyl or methyl);
R₁₃ is a hydrogen or a C₁₋₃ alkyl;
R₁₄ is hydrogen, alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), aryl (such as phenyl, naphthyl, or tolyl), heteroaryl (such as furyl, thienyl, or pyrrolyl), and heterocycloalkyl (such as 5-8, 5, or 6 membered heterocycloalkyl); a combination thereof; or
a combination thereof.

11. The method of claim 10, wherein A₃ is a C₆ aryl or 6-membered heteroaryl having the chemical structure 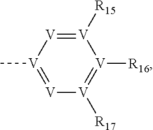
wherein: [symbol]
is the point of attachment with Formula II;
each V is independently a CH or N, wherein there 0, 1, 2, 3, or 4 N groups; and
R₁₅, R₁₆, and R₁₇ are independently selected from halogen (such as Br, F, I, or Cl), —NR₁₃ₐCOR₁₄ₐ (such as —NHCOR₁₄ₐ), hydroxyl, cyano, nitro, amino (such as —NH₂), C₁₋₃ alkyl (such as methyl), C₁₋₄alkoxy (such as methoxy), C₃₋₈ cycloalkyl, C₁₋₂haloalkyl, C₁₋₂haloalkoxy, C₆ aryl, C₈ aryl, 5-8 membered heteroaryl, 3-8 membered heterocycloalkyl (such as 5-8 membered heterocycloalkyl), —CHO, and —COOH, wherein
one of R₁₅, R₁₆, and R₁₇ is a hydrogen; and
one of R₁₅, R₁₆, and R₁₇ is a hydrogen or a C₁₋₆ alkyl (such as C₁₋₃ alkyl or methyl).

12. The method of claim 9, wherein:
A₃ is an aryl (such as C₆ or C₈ aryl), a heteroaryl (such as 5-8 membered heteroaryl), a cycloalkyl (such as a C₃₋₈ cycloalkyl group), or a heterocycloalkyl (such as a 5-8 membered heterocycloalkyl),
A₃ is optionally substituted with one or more (such as 1 or 2) substituents independently selected from halogen (such as Br, F, I, or Cl), —NR₁₁ₐ—CO—R₁₂ₐ (such as —NH—CO—R₁₂ₐ), hydroxyl, cyano, nitro, amino (such as —NH₂), C₁₋₃ alkyl (such as methyl), C₁₋₄ alkoxy (such as methoxy), C₃₋₈ cycloalkyl, C₁₋₂haloalkyl, C₁₋₂haloalkoxy, C₆ or C₈ aryl, 5-8 membered heteroaryl, 3-8 membered heterocycloalkyl (such as 5-8 membered heterocycloalkyl), —CHO, —COOH, and hydrocarbyl having a hydrocarbon chain of 1 to 3 carbon atoms in which the carbon atoms are joined by single, double or triple bonds, and any one carbon atom or any two nonadjacent carbon atoms can be replaced by O, NH, N(C₁-C₄ alkyl), or S, and wherein the hydrocarbyl group is optionally substituted with one or more substituents independently chosen from hydroxyl, oxo, halogen, and amino;
B is CR₁₁R₁₂ or NR;
R₁₁ and R₁₂ are each independently hydrogen, halogen, hydroxyl, cyano, nitro, amino, —CHO, —CONH₂, C₁₋₃ alkyl (such as methyl), C₁₋₃ alkoxy, C₁₋₂haloalkyl, and C₁₋₂haloalkoxy;
R₇, R₈, R₉, and R₁₀ are each independently hydrogen, halogen, hydroxyl, cyano, nitro, amino, —CHO, —CONH₂, —NR₁₃—CO—R₁₄, —CO-aryl (such as —CO—C₆ or C₈ aryl or benzoyl), —CO— alkyl (such as —CO—C₁₋₆ alkyl or acetyl), alkyl (such as C₁₋₆ alkyl or methyl), alkoxy (such as C₁₋₄ alkoxy), cycloalkyl (such as C₃₋₈ cycloalkyl), haloalkyl (such as C₁₋₂haloalkyl), haloalkoxy (such as C₁₋₂haloalkoxy), aryl (such as a C₆ or C₈ aryl), heteroaryl (such as 5-8, 5, or 6 membered heteroaryl, furyl, thienyl, or pyrrolyl), heterocycloalkyl (such as a 3-8 membered heterocycloalkyl), an aryloxy (such as phenoxy), alkylaryl (such as —CH₂—CH₂-phenyl), or a thioaryl (such as thiophenyl);
R₁₃ and R₁₄ are each independently hydrogen, alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), aryl (such as phenyl, naphthyl, or tolyl), heteroaryl (such as furyl, thienyl, or pyrrolyl), and heterocycloalkyl (such as 5-8, 5, or 6 membered heterocycloalkyl);
a combination thereof.

13. The method of claim 12, wherein
A₃ is a C₆ aryl or 6-membered heteroaryl;
A₃ is optionally substituted with 1 or 2 substituents independently selected from halogen (such as Br, F, I, or Cl), —NR₁₁ₐ—CO—R₁₂ₐ (such as —NH—CO—R₁₂ₐ), hydroxyl, cyano, nitro, amino (such as —NH₂), C₁₋₃ alkyl (such as methyl), C₁₋₄ alkoxy (such as methoxy), C₃₋₈ cycloalkyl, C₁₋₂ haloalkyl, C₁₋₂haloalkoxy, C₆ aryl, C₈ aryl, 5-8 membered heteroaryl, 3-8 membered heterocycloalkyl (such as 5-8 membered heterocycloalkyl), —CHO, and —COOH;
R₁₁ₐ is a hydrogen or a C₁₋₃ alkyl;
R₁₂ₐ is a 5-8 membered heteroaryl (such as 5, 6, or 7 membered heteroaryl), 9-11 membered bicyclic heteroaryl (such as 10 membered bicyclic heteroaryl), C₆ monocyclic aryl, C₈ monocyclic aryl, or C₉₋₁₁ bicyclic aryl (such as C₁₀ bicyclic aryl), wherein the R₁₄ₐ is optionally substituted with 1, 2, or 3 substituents independently selected from halogen (such as Cl, Br, I, or F), or C₁₋₃ alkyl (such as methyl), and C₁₋₃ alkoxy (such as methoxy);
R₇, R₉, and R₁₀ are each hydrogen;
A is N;
B is NR₁₁;
R₁₁ is hydrogen or C₁₋₆ alkyl (such as C₁₋₃ alkyl or methyl);
R₁₃ is a hydrogen or a C₁₋₃ alkyl;
R₁₄ is hydrogen, alkyl (such as C₁-C₆ alkyl), alkoxy (such as C₁-C₄ alkoxy), cycloalkyl (such as C₃-C₈ cycloalkyl), haloalkyl (such as C₁-C₂haloalkyl), aryl (such as phenyl, naphthyl, or tolyl), heteroaryl (such as furyl, thienyl, or pyrrolyl), and heterocycloalkyl (such as 5-8, 5, or 6 membered heterocycloalkyl); a combination thereof; or
a combination thereof.

14. The method of claim 13, wherein A₃ is a C₆ aryl or 6-membered heteroaryl having the chemical structure 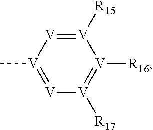
wherein: [symbol]
is the point of attachment with Formula II;
each V is independently a CH or N, wherein there 0, 1, 2, 3, or 4 N groups; and
R₁₅, R₁₆, and R₁₇ are independently selected from halogen (such as Br, F, I, or Cl), —NR₁₃ₐCOR₁₄ₐ (such as —NHCOR₁₄ₐ), hydroxyl, cyano, nitro, amino (such as —NH₂), C₁₋₃ alkyl (such as methyl), C₁₋₄alkoxy (such as methoxy), C₃₋₈ cycloalkyl, C₁₋₂haloalkyl, C₁₋₂haloalkoxy, C₆ aryl, C₈ aryl, 5-8 membered heteroaryl, 3-8 membered heterocycloalkyl (such as 5-8 membered heterocycloalkyl), —CHO, and —COOH, wherein
one of R₁₅, R₁₆, and R₁₇ is a hydrogen; and
one of R₁₅, R₁₆, and R₁₇ is a hydrogen or a C₁₋₆ alkyl (such as C₁₋₃ alkyl or methyl).

15. The method of claim 12, wherein either (i) or (ii):
(i) A₃ is an aryl (such as a monocyclic aryl or bicyclic aryl), a heteroaryl (such as a monocyclic heteroaryl or bicyclic heteroaryl), a cycloalkyl (such as a C₃₋₈ cycloalkyl group), or a heterocycloalkyl (such as a 5-8 membered heterocycloalkyl), wherein:
(a) one carbon atom (such as R₁₅, R₁₆, or R₁₇) is —NR₁₁ₐ—CO—R₁₂ₐ (such as —NH—CO—R₁₂ₐ), cycloalkyl (such as C₃-C₈ cycloalkyl), aryl (such as a C₆ or C₈ aryl), heteroaryl (such as a 5-8 membered heteroaryl), heterocycloalkyl (such as a 3-8 or 5-8 membered heterocycloalkyl), —CHO, or —COOH; and
(b) a second carbon atom (such as R₁₅, R₁₆, or R₁₇) is optionally substituted with a halogen (such as Br, F, I, or Cl), hydroxyl, cyano, nitro, amino (such as —NH₂), alkyl (such as C₁₋₆ or C₁₋₃ alkyl or methyl), alkoxy (such as C₁₋₄ alkoxy or methoxy), haloalkoxy (such as C₁₋₂ haloalkoxy); or
(ii) R₇, R₈, R₉, and R₁₀ are each independently selected from hydrogen, halogen, hydroxyl, cyano, nitro, amino, —CHO, —CONH₂, —CO-alkyl (such as —CO—C₁₋₆ alkyl or acetyl), alkyl (such as C₁₋₆ alkyl or methyl), alkoxy (such as C₁₋₄ alkoxy), cycloalkyl (such as C₃₋₈ cycloalkyl), haloalkyl (such as C₁₋₂haloalkyl), and haloalkoxy (such as C₁₋₂haloalkoxy),
wherein R₈ or R₉ (such as R₈) is —NR₁₃—CO—R₁₄, —CO-aryl (such as —CO—C₆ or C₈ aryl or benzoyl), —CO-heteroaryl (such as 5-8, 5 or 6 membered heteroaryl), aryl (such as a C₆ or C₈ aryl), heteroaryl (such as 5-8, 5, or 6 membered heteroaryl, furyl, thienyl, or pyrrolyl), heterocycloalkyl (such as a 3-8 membered heterocycloalkyl), an aryloxy (such as phenoxy), alkylaryl (such as —CH₂—CH₂-phenyl), and a thioaryl (such as thiophenyl).

16. The method of claim 9, wherein either (i) or (ii):
(i) A₃ is an aryl (such as a monocyclic aryl or bicyclic aryl), a heteroaryl (such as a monocyclic heteroaryl or bicyclic heteroaryl), a cycloalkyl (such as a C₃₋₈ cycloalkyl group), or a heterocycloalkyl (such as a 5-8 membered heterocycloalkyl), wherein:
(a) one carbon atom is —NR₁₁ₐ—CO—R₁₂ₐ (such as —NH—CO—R₁₂ₐ), cycloalkyl (such as C₃-C₈ cycloalkyl), aryl (such as a C₆ or C₈ aryl), heteroaryl (such as a 5-8 membered heteroaryl), heterocycloalkyl (such as a 3-8 or 5-8 membered heterocycloalkyl), —CHO, or —COOH; and
(b) a second carbon atom is optionally substituted with a halogen (such as Br, F, I, or Cl), hydroxyl, cyano, nitro, amino (such as —NH₂), alkyl (such as C₁₋₆ or C₁₋₃ alkyl or methyl), alkoxy (such as C₁₋₄ alkoxy or methoxy), haloalkoxy (such as C₁₋₂haloalkoxy); or
(ii) R₇, R₈, R₉, and R₁₀ are each independently selected from hydrogen, halogen, hydroxyl, cyano, nitro, amino, —CHO, —CONH₂, —CO-alkyl (such as —CO—C₁₋₆ alkyl or acetyl), alkyl (such as C₁₋₆ alkyl or methyl), alkoxy (such as C₁₋₄ alkoxy), cycloalkyl (such as C₃₋₈ cycloalkyl), haloalkyl (such as C₁₋₂haloalkyl), and haloalkoxy (such as C₁₋₂haloalkoxy),
wherein R₈ or R₉ (such as R₈) is —NR₁₃—CO—R₁₄, —CO-aryl (such as —CO—C₆ or C₈ aryl or benzoyl), —CO-heteroaryl (such as 5-8, 5 or 6 membered heteroaryl), aryl (such as a C₆ or C₈ aryl), heteroaryl (such as 5-8, 5, or 6 membered heteroaryl, furyl, thienyl, or pyrrolyl), heterocycloalkyl (such as a 3-8 membered heterocycloalkyl), an aryloxy (such as phenoxy), alkylaryl (such as —CH₂—CH₂-phenyl), and a thioaryl (such as thiophenyl).

17. The method of claim 16, wherein A₃ is a C₆ aryl or 6-membered heteroaryl having the chemical structure 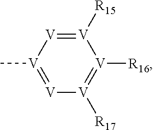
wherein: [symbol]
is the point of attachment with Formula II;
each V is independently a CH or N, wherein there 0, 1, 2, or 3 N groups; and
R₁₅, R₁₆, and R₁₇ are independently selected from halogen (such as Br, F, I, or Cl), —NR₁₃ₐCOR₁₄ₐ (such as —NHCOR₁₄ₐ), hydroxyl, cyano, nitro, amino (such as —NH₂), C₁₋₃ alkyl (such as methyl), C₁₋₄alkoxy (such as methoxy), C₃₋₈ cycloalkyl, C₁₋₂haloalkyl, C₁₋₂haloalkoxy, C₆ aryl, C₈ aryl, 5-8 membered heteroaryl, and 3-8 membered heterocycloalkyl (such as 5-8 membered heterocycloalkyl), —CHO, and —COOH, wherein
one of R₁₅, R₁₆, and R₁₇ is a hydrogen; and
one of R₁₅, R₁₆, and R₁₇ is a hydrogen or a C₁₋₆ alkyl (such as C₁₋₃ alkyl or methyl).

18. The method of claim 9, wherein the compound is selected from compounds 51-67.

19. A method of treating or preventing a disease or disorder, or symptom associated with a picornavirus infection in a subject, the method comprising:
providing and administering a therapeutically effective amount of a compound selected from 1-[bis(4-fluorophenyl)methoxy]-3-(5,6-difluoro-1H-1,3-benzodiazol-1-yl)propan-2-ol (compound 108), (4,6-diamino-1,3,5-triazin-2-yl)methyl 3-[5-fluoro-2-(4-fluorophenyl)-1H-indol-3-yl]propanoate (compound 111), 4-(2-{1-[(2-chlorophenyl)methyl]-1H-indol-3-yl}ethenyl)-6-(trifluoromethyl)-1,2-dihydropyrimidin-2-one (compound 139), and N-(3-chlorophenyl)-2-[8-(3-fluorophenoxy)-3-oxo-2H,3H-[1,2,4]triazolo[4,3-a]pyrazin-2-yl]acetamide (compound 167),
or a pharmaceutically acceptable salt thereof, or a composition comprising the same to the subject,
wherein the method is effective in treating, preventing, or ameliorating the disease or disorder associated with a picornaviruses infection, or at least one symptom of the disease or disorder associated with a picornavirus infection.
